(19) United States
(12) Patent Application Publication
(10) Pub. No.: US 2023/0360779 A1
(43) Pub. Date: Nov. 9, 2023

(54) SYSTEMS AND METHODS FOR USING A DISTRIBUTED LEDGER TO MANAGE KNOWLEDGE IN A HEALTHCARE ECOSYSTEM

(71) Applicant: Healthpointe Solutions, Inc., Austin, TX (US)

(72) Inventors: NATHAN GNANASAMBANDAM, Irvine, CA (US); MARK HENRY ANDERSON, Newport Coast, CA (US)

(73) Assignee: Healthpointe Solutions, Inc., Austin, TX (US)

(21) Appl. No.: 17/905,112

(22) PCT Filed: Feb. 9, 2021
(86) PCT No.: PCT/US21/17245

§ 371 Date: Aug. 26, 2022

Related U.S. Application Data
(60) Provisional application No. 62/984,062, filed on Mar. 2, 2020.

(51) Int. Cl. G16H 40/20; G06Q 20/36

(52) U.S. Cl. CPC G16H 40/20; G06Q 20/3678

(57) ABSTRACT

A node that is part of a network of nodes with access to a distributed ledger may receive a transaction request to perform one or more operations associated with the distributed ledger, wherein the one or more operations include causing a record of a transaction to be stored using the distributed ledger. The node may determine, based on credential information associated with the transaction request, whether to allow the one or more operations to be performed. The node may perform the one or more operations based on determining to allow the one or more operations to be performed. The one or more operations may include adding the record of the transaction to the distributed ledger. At least a subset of the content may be included in the record. The node may cause one or more other nodes, of the network of nodes, to have access to the distributed ledger.

### CROSS-REFERENCE TO RELATED APPLICATIONS

This application claims the benefit of U.S. Provisional Application Ser. No. 62/984,062 filed Mar. 2, 2020 titled “Systems and Methods for Using a Distributed Ledger to Manage Knowledge in a Healthcare Ecosystem,” which provisional application is incorporated by reference herein as if reproduced in full below.

### BACKGROUND

A blockchain is a distributed database that maintains a continuously-growing list of records, called blocks, that may be linked together to form a chain. Each block in the blockchain may contain a timestamp and a link to a previous block and/or record. The blocks may be secured from tampering and revision. In addition, a blockchain may include a secure transaction ledger database shared by parties participating in an established, distributed network of computers. A blockchain may store a record of a transaction (e.g., an exchange or transfer of information) that occurs in the network, thereby reducing or eliminating the need for trusted/centralized third parties. In some cases, the parties participating in a transaction may not know the identities of any other parties participating in the transaction but may securely exchange information. Further, the distributed ledger may correspond to a record of consensus with a cryptographic audit trail that is maintained and validated by a set of independent computers.

### SUMMARY

This section provides a general summary of the present disclosure and is not a comprehensive disclosure of its full scope or all of its features, aspects, and objectives.

Disclosed herein are implementations of a method that includes receiving, by a node that is part of a network of nodes with access to a distributed ledger, a transaction request to perform one or more operations associated with the distributed ledger. The one or more operations may include causing a record of a transaction to be stored using the distributed ledger. The method may include determining, based on credential information associated with the transaction request, whether to allow the one or more operations to be performed. The method may include performing the one or more operations based on determining to allow the one or more operations to be performed. The one or more operations may include adding the record of the transaction to the distributed ledger. At least a subset of the content may be included in the record. The method may include causing one or more other nodes, of the network of nodes, to have access to the distributed ledger.

Also disclosed herein are implementations of a node that includes one or more processors and one or more memories including instructions that, when executed by the one or more processors, cause the one or more processors to receive a transaction request to perform one or more operations associated with a distributed ledger. The node may be part of a network of nodes with access to the distributed ledger. The one or more operations may include causing a record of a transaction to be stored using the distributed ledger. The one or more processors may determine, based on credential information associated with the transaction request, whether to allow the one or more operations to be performed. The one or more processors may perform the one or more operations based on determining to allow the one or more operations to be performed. The one or more operations may include adding the record of the transaction to the distributed ledger. At least a subset of the content may be included in the record. The one or more processors may cause one or more other nodes, of the network of nodes, to have access to the distributed ledger.

Also disclosed herein is a non-transitory computer-readable medium storing instructions that, when executed by one or more processors, cause the one or more processors to receive a transaction request to perform one or more operations associated with a distributed ledger. The node may be part of a network of nodes with access to the distributed ledger. The one or more operations may include causing a record of a transaction to be stored using the distributed ledger. The one or more instructions, when executed by the one or more processors, may cause the one or more processors to determine, based on credential information associated with the transaction request, whether to allow the one or more operations to be performed. The one or more instructions, when executed by the one or more processors, may cause the one or more processors to perform the one or more operations based on determining to allow the one or more operations to be performed. The one or more operations may include adding the record of the transaction to the distributed ledger. At least a subset of the content may be included in the record. The one or more instructions, when executed by the one or more processors, may cause the one or more processors to cause one or more other nodes, of the network of nodes, to have access to the distributed ledger.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 illustrates, in block diagram form, a system architecture 100 that can be configured to provide a population health management service, in accordance with various embodiments.

FIG. 2 shows additional details of a knowledge cloud, in accordance with various embodiments.

FIG. 3 shows an example subject matter ontology, in accordance with various embodiments.

FIG. 4 shows aspects of a conversation, in accordance with various embodiments.

FIG. 5 shows a cognitive map or “knowledge graph”, in accordance with various embodiments.

FIG. 6 shows an example method, in accordance with various embodiments.

FIGS. 7A, 7B, and 7C show example methods, in accordance with various embodiments.

FIGS. 8A, 8B, 8C, and 8D show aspects of a user interface, in accordance with various embodiments.

FIGS. 9A and 9B show aspects of a conversational stream, in accordance with various embodiments.

FIG. 10 shows aspects of a conversational stream, in accordance with various embodiments.

FIG. 11 shows aspects of an action calendar, in accordance with various embodiments.

FIG. 12 shows aspects of a feed, in accordance with various embodiments.

FIG. 13 shows aspects of a hyper-local community, in accordance with various embodiments.

FIG. 14 illustrates a detailed view of a computing device that can represent the computing devices of FIG. 1 used to implement the various platforms and techniques described herein, according to some embodiments.

FIG. 15 shows an example method, in accordance with various embodiments.

FIG. 16 shows an example method, in accordance with various embodiments.

FIG. 17 shows an example method, in accordance with various embodiments.

FIG. 18 shows a therapeutic paradigm logical framework, in accordance with various embodiments

FIG. 19 shows an example method, in accordance with various embodiments.

FIG. 20 shows a paradigm logical framework, in accordance with various embodiments.

FIG. 21 shows an example method, in accordance with various embodiments.

FIG. 22 shows an example method, in accordance with various embodiments.

FIG. 23 shows a distributed network of nodes each maintaining a copy of a distributed ledger to manage content, in accordance with various embodiments.

FIG. 24 shows an example distributed ledger, in accordance with various embodiments.

FIG. 25A-25D show a knowledge graph, an updated knowledge graph, a patient graph, and a representation of at least a portion of a care plan, in accordance with various embodiments.

FIG. 25B shows the cognitive map or “knowledge graph” of FIG. 25A evolved to a second knowledge stage, in accordance with various embodiments.

FIG. 26 shows a general process for performing transaction requests on a distributed ledger by various nodes in a healthcare ecosystem, in accordance with various embodiments.

FIG. 27 shows an example content stored on the distributed ledger, in accordance with various embodiments.

FIG. 28 shows an example method, in accordance with various embodiments.

FIG. 29 shows an example search for content, in accordance with various embodiments.

FIG. 30 shows an example method, in accordance with various embodiments.

FIG. 31 shows example updated content stored in the distributed ledger, in accordance with various embodiments.

FIG. 32 shows an example method, in accordance with various embodiments.

FIGS. 33A-33E are diagrams of one or more example embodiments described herein.

FIG. 34 shows a flowchart of an example method for managing content pertaining to healthcare in a distributed ledger, in accordance with various embodiments.

### DETAILED DESCRIPTION

The following discussion is directed to various embodiments of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Population health management entails aggregating patient data across multiple health information technology resources, analyzing the data with reference to a single patient, and generating actionable items through which care providers can improve both clinical and financial outcomes. In some instances, coordinating health services to perform the actionable items among multiple entities in a healthcare ecosystem can be a daunting, inefficient, and/or cumbersome task. Further, various health providers may use different care plans for treating illnesses or health issues of their patients. The care plan used by a first physician may not be as effective as the care plan used by another physician. However, the first physician may not be aware of the more effective care plan. Further, it may be difficult to verify the source of certain content, such as evidence-based guidelines, clinical processes, clinical trials, care plans, etc., in a verifiable manner.

There are numerous entities involved in transactions associated with a healthcare ecosystem. For example, the entities may include patients (consumers), medical personnel (e.g., physicians, nurses, pharmacists, dentists, optometrists, orthodontists, etc.), insurance providers, clinics, hospitals, pharmacies, professional associations, government agencies, and/or the like. Example transactions in the healthcare ecosystem may include a patient requesting content pertaining to healthcare, a physician providing content pertaining to healthcare, a physician verifying content pertaining to healthcare, a physician updating content pertaining to healthcare, a physician deleting content pertaining to healthcare, and/or the like. The content may include evidence-based guidelines (e.g., published by one or more physicians, a professional association, and/or government agency), knowledge representations, clinical studies, clinical processes, clinical techniques, care plans, and/or the like. The content may be presented in one or more documents (e.g., a word processing document, a spreadsheet document, a slideshow document), videos, images, and/or the like. In some instances, the content may be a combination of information presented in different types of documents (e.g., a video embedded in a word processing document including text).

Medical personnel entities, such as physicians, may generate content (e.g., care plans) for particular medical conditions (e.g., illnesses, diseases, etc.). The care plans may include steps for a patient to take to recover from the medical condition and/or to reduce symptoms of the medical condition. For example, the steps may relate to a type of medication to take and a schedule for taking the medication, an exercise plan, a diet plan, a rest plan, and/or the like for a patient. The care plans may be individually tailored for characteristics (e.g., age, weight, height, gender, active level, etc.) and/or nuances of each patient.

In some instances, the physicians may not share the care plans with one another in an effort to earn business of patients by offering a proprietary care plan. Physician A may have his own care plan for diabetes that is different than another care plan that is used by physician B. The efficacies of the care plans may vary. For example, if followed, physician A's care plan may provide better results (e.g., cures an illness, faster recovery time, reduces symptoms, etc.) for a patient than physician B's care plan. Physician B may desire to use physician A's care plan but may not have access to physician A's care plan. Currently, there is no reliably secure and efficient technique to share the content between physicians, or a reliably secure and efficient technique for other physicians to review, verify, and/or modify the care plans. It may be advantageous to the physicians to profit from their knowledge that is encompassed in their unique care plans. Conventional systems do not provide a way for the care plans to be monetized in a secure and verifiable way such that physicians may purchase and/or acquire access rights to desired care plans.

Accordingly, the disclosed embodiments generally relate to techniques for managing content (e.g., evidence-based guideline, knowledge representations, clinical studies, clinical processes, clinical techniques, care plans, etc.) using a blockchain. A blockchain may refer to an immutable ledger for storing records of transactions.

The cognitive intelligence platform integrates and consolidates data/information from various sources and entities and provides a population health management service. In some embodiments, at least some of the data/information from the various sources and entities may be stored in the blockchain. The blockchain may be maintained by a distributed network of nodes. In some embodiments, a consensus protocol may be used by the nodes to determine whether to allow transactions to be performed and groups the transactions into records that are stored as blocks of the blockchain.

There are different kinds of blockchains, such as permission-less and permissioned. In a permission-less blockchain, any entity may participate without an identity. In a permissioned blockchain, each entity that participates in the blockchain is identified and known. An example of a permissioned blockchain is a distributed ledger (e.g., a hyperledger). The permissions cause the participating nodes to view only the appropriate records of transactions in the distributed ledger. Programmable logic may be implemented as rules and/or smart contracts that are executed on the distributed ledger. In some embodiments, the rules may be analytics-based and may specify scenarios when updates to the distributed ledger are to be made by the various entities of the healthcare ecosystem. Using the analytics-based rules may make each node an active participant by updating the distributed ledger at specified times.

The distributed ledger may provide a verifiable trace of proof that the content stored on the distributed ledger is associated with entities having authorized credentials (e.g., medical license) to facilitate more efficient verification of the information, among other things. The distributed ledger may provide a secure chain of record that is used to enhance the efficiency and/or security of the knowledge management process in the healthcare ecosystem. An objective process of administering and managing clinical knowledge can be achieved using the distributed ledger in disclosed embodiments. A user's experience using a computer may be improved using the disclosed embodiments by verifying the source of content in a secure manner, such that the user is confident that the content is trustworthy because it was written by a medical personnel entity having valid authorization information, has been recently updated by a medical personnel entity having valid authorization information, and/or was vetted by other medical personnel entities having valid authorization information. Further, network, processor, and/or memory resources may be reduced using the disclosed techniques by the distributed ledger returning ranked content that is (i) written by a medical personnel entity having a stellar reputation, (ii) viewed by a threshold number of medical personnel entities, and/or (iii) verified as being valid by a threshold number of medical personnel entities because the user may select content initially presented based on one or more of these factors without performing additional searches.

Each entity in the healthcare ecosystem may register as a node in a distributed, decentralized network. Registering a node for an entity may involve a record of a transaction that is added to the distributed ledger. Each node may maintain a respective copy of the distributed ledger as a shared single source of truth. During registration, each entity may provide certain information pertaining to the entity to be maintained by the distributed ledger at the nodes. For example, a physician may register as a node and may provide information (e.g., National Provider Identifier (NPI), license number, date licensed, date license last updated, etc.) pertaining to their authorization information, specialty of medical practice, location of practice, and any other information relevant to practicing in the healthcare ecosystem. A pharmacist may register as a node and may provide information (e.g., license number, date licensed, date license last updated, etc.) pertaining to their authorization information, location of practice, and any other information relevant to practicing in the healthcare ecosystem. A patient may register as a node and may provide personal information (e.g., driver's license number, social security number, name, insurance provider number, type of insurance, address, medical records, allergies, etc.) that enables verifying their identity and establishing a user profile, among other things. A non-patient user may also register as a node by providing personal information. A news organization may also register as a node by providing authorization information associated with its entity type.

In some embodiments, just the entities that are registered as nodes may add content to the distributed ledger. For example, in the context of a social media forum, using the disclosed techniques may prevent a user without a node to publish misleading and potentially untrue information on the social media forum.

A computer-implemented application may be accessible on a computing device of each entity. The application may be written in computer instructions that are stored on one or more memory devices of the computing device and executable by one or more processing devices of the computing device. In some embodiments, the application may be a stand-alone application that is installed on the computing device, while in other embodiments, the application may be executable via another application (e.g., a website in a web browser).

A medical personnel entity may use the application to store documents on the distributed ledger. For example, a medical personnel entity may use the application to submit a transaction request to perform an operation on the distributed ledger. The operation may include storing content (e.g., knowledge representation, care plan, etc.) on the distributed ledger. One or more rules of the distributed ledger may be executed prior to allowing the operation to be performed. The rules may be logic implemented in computer instructions of a rules engine, a smart contract, and/or the like. One of the rules may determine whether the medical personnel entity that submitted the transaction request is associated with valid authorization information (e.g., medical degree, medical license number). Another rule may determine whether the content includes any portions that are new relative to other content stored on the distributed ledger. For example, the rule may prevent duplicated knowledge from being added to the distributed ledger. That is, at least a portion of the content being added may be required to be new and unique and not disclosed by other content on the distributed ledger.

Each entity may use the application to search for desired content, such as care plans, on the distributed ledger. The content may be associated with various access rights. For example, when stored, the content can be set to public such that anyone using the application can obtain the content. The content may be set to private such that a user has to have a certain access right to obtain the content. In some embodiments, a user may purchase an access right to particular content and the author of that particular content may profit. In some embodiments, users that are part of a same organization (e.g., hospital) may have access rights to content associated with the users of that organization.

The distributed ledger enables tracing the content to a source so a user can verify that the content was generated by a medical personnel entity having valid authorization information, for example. Further, the distributed ledger may record how many licensed medical personnel entities have viewed a particular content, have verified the particular content, have edited the particular content, a timestamp of the latest update to the particular content, whether the content is still valid, and/or the like. A user may view a time series of when the content was created and when the content was updated over time. Further, a date at which the content is required to be updated may also be presented by the application. The distributed ledger may enable content to evolve with additional content over time and provides security to ensure that the content is modified by licensed professionals.

The cognitive intelligence platform has the ability to extract concepts, relationships, and draw conclusions from a given text posed in natural language (e.g., a passage, a sentence, a phrase, and a question) by performing conversational analysis which includes analyzing conversational context. For example, the cognitive intelligence platform has the ability to identify the relevance of a posed question to another question.

The benefits provided by the cognitive intelligence platform, in the context of healthcare, include freeing up physicians from focusing on day to day population health management. Thus a physician can focus on her core competency—which includes disease/risk diagnosis and prognosis and patient care. The cognitive intelligence platform provides the functionality of a health coach and includes a physician's directions in accordance with the medical community's recommended care protocols and also builds a systemic knowledge base for health management. The cognitive intelligence platform may leverage the information stored in the distributed ledger to recommend certain actions be taken by a patient. For example, using the distributed ledger, the recommended actions may include setting up a consultation with a physician having valid authorization information at a location near the patient (e.g., based on geolocations of devices of the entities).

The cognitive intelligence platform may implement an intuitive conversational cognitive agent that engages in a question and answering system that is human-like in tone and response. The described cognitive intelligence platform endeavors to compassionately solve goals, questions and challenges. Further, the cognitive intelligence platform may use a distributed ledger to manage knowledge between entities in a healthcare ecosystem more efficiently and/or securely. The described methods and systems are described as occurring in the healthcare space, though other areas are also contemplated.

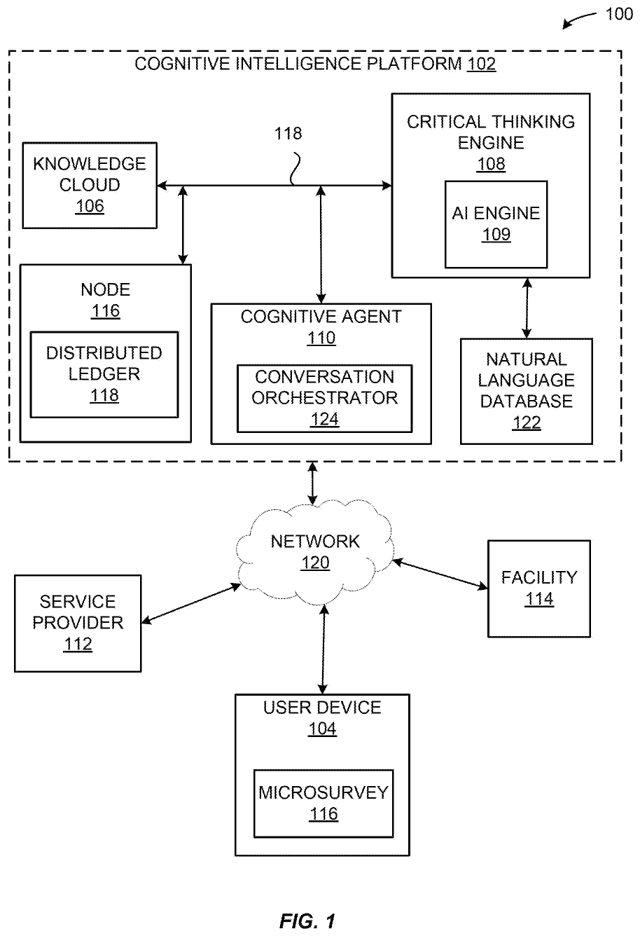

FIG. 1 shows a system architecture 100 that can be configured to provide a population health management service, in accordance with various embodiments. Specifically, FIG. 1 illustrates a high-level overview of an overall architecture that includes a cognitive intelligence platform 102 communicably coupled to a user device 104. The cognitive intelligence platform 102 includes several computing devices, where each computing device, respectively, includes at least one processor, at least one memory, and at least one storage (e.g., a hard drive, a solid-state storage device, a mass storage device, and a remote storage device). The individual computing devices can represent any form of a computing device such as a desktop computing device, a rack-mounted computing device, and a server device. The foregoing example computing devices are not meant to be limiting. On the contrary, individual computing devices implementing the cognitive intelligence platform 102 can represent any form of computing device without departing from the scope of this disclosure.

The several computing devices work in conjunction to implement components of the cognitive intelligence platform 102 including: a knowledge cloud 106; a critical thinking engine 108; an artificial intelligence engine 109 (“AI Engine” in FIG. 1), a natural language database 122; a cognitive agent 110; and a node 116. The cognitive intelligence platform 102 is not limited to implementing only these components, or in the manner described in FIG. 1. That is, other system architectures can be implemented, with different or additional components, without departing from the scope of this disclosure. The example system architecture 100 illustrates one way to implement the methods and techniques described herein.

The node 116 represents a single computing device in a distributed blockchain network of nodes 116 (also referred to as a distributed ledger fabric herein) of the cognitive intelligence platform 102. A permissioned type of blockchain, referred to as a distributed ledger 118, may be implemented and a respective copy of the distributed ledger 118 may be stored on a respective node 116. The nodes 116 may represent any suitable entity in a healthcare ecosystem. For example, some of the entities may include a service provider 112 (e.g., medical personnel entity, such as a physician, dentist, pharmacist, optometrist, orthodontic, nurse, etc.), a facility 114 (e.g., medical facility entity), a patient entity, and/or the like. Each entity may be associated with a respective computing device that they use to register as a node on the blockchain network and request transactions to be performed using the distributed ledger 118.

In a permissioned blockchain, such as the distributed ledger 118, the entities register by providing certain information to the distributed ledger. Based on one or more rules associated with the distributed ledger 118, the entity may register as a node 116 on the blockchain network and be provided with authentication information that is used to identify the entities when they request transactions to be performed on the distributed ledger 118. The rules may be executable software modules that are installed in the distributed ledger 118 itself. In some instances, when a user sends a transaction request to the distributed ledger 118, the distributed ledger 118 may invoke the rules, which perform functions depending on the type of transaction being requested. In addition, the nodes 116 may employ a consensus protocol whereby the nodes 116 communicate with each other to determine whether to allow the transaction to be performed to modify the distributed ledger 118.

The entities use computing devices to send requests to perform transactions using the distributed ledger 118 to the cognitive intelligence platform 102. The transactions may include performing various operations. When applicable rules and/or the consensus protocol is satisfied, the operation in the requested transactions may be completed and a record of the transaction may be added to the distributed ledger 118. The transactions may include storing content (e.g., a care plan) on the distributed ledger 118, storing updated content (e.g., an updated care plan that includes a modification relative to a previously stored care plan), verifying content on the distributed ledger 118, providing content to a user device 104, and/or the like. In some instances, the transactions may not be altered or removed, thereby providing an immutable quality to the distributed ledger 118. Further, cryptography may be used to secure the distributed ledger 118 and the messages between the nodes 116 of the blockchain network and/or the computing devices requesting the transactions. In some embodiments, just the authorized entities are allowed to perform the transactions on the distributed ledger 118, and in some instances, just the appropriate entities are allowed to view details of particular transactions in the distributed ledger 118.

In some embodiments, a transaction request to register as a node 116 may be a type of transaction that is stored using the distributed ledger 118. The entities may send the requests to register as a node 116 using the distributed ledger 118, and the requests include certain information pertaining to the entities. For example, a medical personnel entity may provide authorization information, such as a medical license number. If the rules and/or the consensus protocol is satisfied, the entity may be associated with a node 116. Further, the distributed ledger 118 may be updated by adding a block storing a record of the transaction including the information pertaining to the entity that is associated with the node 116. The updated distributed ledger 118 may be stored at the node for the entity. In some embodiments, the copies of the other distributed ledgers 118 at the other nodes 116 in the blockchain network may be updated with the new transaction. Further, when the entity is registered as a node 116, the computing device associated with that entity may be provided with authentication information for that entity. The computing device may use the authentication information to make subsequent requests to the distributed ledger 118. The authentication information may be a username, password, hash code, or the like that uniquely identify an identity of the entity. The entities (e.g., physician, patient, etc.) may use a software application running on a computing device to submit the transaction requests to the distributed ledger 118.

The distributed ledger 118 may be used as a verifiable trace of proof to determine that the source of certain content (e.g., care plans) were generated and provided by licensed entities (e.g., medical doctors) having valid authorization information. The distributed ledger 118 may execute its rules when a request to upload content is received, when a request to modify a stored content is received, when a request to verify content is received, when a request to view content is received, and/or the like. In some embodiments, the rules may require that the entity be associated with the authentication information, the entity be associated with the authorization information, and/or the content that is requested to be added is new prior to allowing the transaction to be performed.

The knowledge cloud 106 represents a set of instructions executing within the cognitive intelligence platform 102 that implement a database configured to receive inputs from several sources and entities. For example, some of the sources and entities include a service provider 112, a facility 114, and a microsurvey 116—each described further below.

The critical thinking engine 108 represents a set of instructions executing within the cognitive intelligence platform 102 that execute tasks using artificial intelligence, such as recognizing and interpreting natural language (e.g., performing conversational analysis), and making decisions in a linear manner (e.g., in a manner similar to how the human left brain processes information). Specifically, an ability of the cognitive intelligence platform 102 to understand natural language is powered by the critical thinking engine 108. In various embodiments, the critical thinking engine 108 includes a natural language database 122. The natural language database 112 includes data curated over at least thirty years by linguists and computer data scientists, including data related to speech patterns, speech equivalents, and algorithms directed to parsing sentence structure.

Furthermore, the critical thinking engine 108 is configured to deduce causal relationships given a particular set of data, where the critical thinking engine 108 is capable of taking the individual data in the particular set, arranging the individual data in a logical order, deducing a causal relationship between each of the data, and drawing a conclusion. The ability to deduce a causal relationship and draw a conclusion (referred to herein as a “causal” analysis) is in direct contrast to other implementations of artificial intelligence that mimic the human left brain processes. For example, the other implementations can take the individual data and analyze the data to deduce properties of the data or statistics associated with the data (referred to herein as an “analytical” analysis). However, these other implementations are unable to perform a causal analysis—that is, deduce a causal relationship and draw a conclusion from the particular set of data. As described further below—the critical thinking engine 108 is capable of performing both types of analysis: causal and analytical.

In some embodiments, the critical thinking engine 108 includes an artificial intelligence engine 109 that uses one or more machine learning models. The one or more machine learning models may be generated by a training engine and may be implemented in computer instructions that are executable by one or more processing device of the training engine, the artificial intelligence engine 109, another server, and/or the user device 104. To generate the one or more machine learning models, the training engine may train, test, and validate the one or more machine learning models. The training engine may be a rackmount server, a router computer, a personal computer, a portable digital assistant, a smartphone, a laptop computer, a tablet computer, a camera, a video camera, a netbook, a desktop computer, a media center, or any combination of the above. The one or more machine learning models may refer to model artifacts that are created by the training engine using training data that includes training inputs and corresponding target outputs. The training engine may find patterns in the training data that map the training input to the target output, and generate the machine learning models that capture these patterns.

The one or more machine learning models may be trained to generate one or more knowledge graphs each pertaining to a particular medical condition. The knowledge graphs may include individual elements (nodes) that are linked via predicates of a logical structure. The logical structure may use any suitable order of logic (e.g., higher order logic and/or Nth order logic). Higher order logic may be used to admit quantification over sets that are nested arbitrarily deep. Higher order logic may refer to a union of first-, second-, third, . . . , Nth order logic. Clinical-based evidence, clinical trials, physician research, and the like that includes various information (e.g., knowledge) pertaining to different medical conditions may be input as training data to the one or more machine learning models. The information may pertain to facts, properties, attributes, concepts, conclusions, risks, correlations, complications, etc. of the medical conditions. Keywords, phrases, sentences, cardinals, numbers, values, objectives, nouns, verbs, concepts, and so forth may be specified (e.g., labeled) in the information such that the machine learning models learn which ones are associated with the medical conditions. The information may specify predicates that correlates the information in a logical structure such that the machine learning models learn the logical structure associated with the medical conditions.

In some embodiments, the one or more machine learning models may be trained to transform input unstructured data (e.g., patient notes) into cognified data using the knowledge graph and the logical structure. The machine learning models may identify indicia in the unstructured data and compare the indicia to the knowledge graphs to generate possible health related information (e.g., tags) pertaining to the patient. The possible health related information may be associated with the indicia in the unstructured data. The one or more machine learning models may also identify, using the logical structure, a structural similarity of the possible health related information and a known predicate in the logical structure. The structural similarity between the possible health related information and the known predicate may enable identifying a pattern (e.g., treatment patterns, education and content patterns, order patterns, referral patterns, quality of care patterns, risk adjustment patterns, etc.). The one or more machine learning models may generate the cognified data based on the structural similarity and/or the pattern identified. Accordingly, the machine learning models may use a combination of knowledge graphs, logical structures, structural similarity comparison mechanisms, and/or pattern recognition to generate the cognified data. The cognified data may be output by the one or more trained machine learning models.

The cognified data may provide a summary of the medical condition of the patient. A diagnosis of the patient may be generated based on the cognified data. The summary of the medical condition may include one or more insights not present in the unstructured data. The summary may identify gaps in the unstructured data, such as treatment gaps (e.g., should prescribe medication, should provide different medication, should change dosage of medication, etc.), risk gaps (e.g., the patient is at risk for cancer based on familial history and certain lifestyle behaviors), quality of care gaps (e.g., need to check-in with the patient more frequently), and so forth. The summary of the medical condition may include one or more conclusions, recommendations, complications, risks, statements, causes, symptoms, etc. pertaining to the medical condition. In some embodiments, the summary of the medical condition may indicate another medical condition that the medical condition can lead to. Accordingly, the cognified data represents intelligence, knowledge, and logic cognified from unstructured data.

In some embodiments, the cognified data may be reviewed by physicians and the physicians may provide feedback pertaining to whether or not the cognified data is accurate. Also, the physicians may provide feedback pertaining to whether or not the diagnosis generated using the cognified data is accurate. This feedback may be used to update the one or more machine learning models to improve their accuracy.

In some embodiments, the cognified data may be stored using a distributed ledger 118 (e.g., as part of a patient graph that is stored as a record in the distributed ledger 118, as a unique record in the distributed ledger 118, etc.). In some embodiments, a care plan may be stored using the distributed ledger, as described elsewhere herein. In some embodiments, a portion of the care plan may be stored using the distributed ledger. In some embodiments, the stored portion of the care plan may be selected based on a determination that the portion is not already being stored by the distributed ledger.

In some embodiments, the AI engine may train one or more models to generate output values indicative of at least a portion of content included in a care plan. For example, unstructured data (e.g., patient notes, etc.) may be provided as input values to the one or more models and the one or more models may be trained to compare the input values with one or more knowledge graphs and/or patient graphs to generate the output values. The outputs values may be generated based on similarities and/or patterns identified between the input values and content included in the knowledge graphs and/or the patient graphs.

In some embodiments, the AI engine may train one or more models to determine whether at least a portion of content included in a care plan is already being stored using the distributed ledger 118. For example, if a care plan includes content already stored using the distributed ledger 118, but the stored content includes synonym terms or different nomenclature to express the same concepts, then a natural language comparison of content in the care plan to stored content may be insufficient to determine whether the distributed ledger 118 is already storing the content of the care plan. Consequently, and as will be described further herein, the AI engine may train one or more models to process the one or more knowledge graphs and/or patient graphs to determine whether content included in the care plan is already being stored using the distributed ledger 118. By using the one or more models to make this determination, the cognitive intelligence platform 102 may store only the new content of the care plan that is not already being stored via the distributed ledger 118, thereby conserving memory resources relative to storing duplicative content. Additional information regarding the AI engine is provided further herein.

The cognitive agent 110 represents a set of instructions executing within the cognitive intelligence platform 102 that implement a client-facing component of the cognitive intelligence platform 102. The cognitive agent 110 is an interface between the cognitive intelligence platform 102 and the user device 104. And in some embodiments, the cognitive agent 110 includes a conversation orchestrator 124 that determines pieces of communication that are presented to the user device 104 (and the user). When a user of the user device 104 interacts with the cognitive intelligence platform 102, the user interacts with the cognitive agent 110. The several references herein, to the cognitive agent 110 performing a method, can implicate actions performed by the critical thinking engine 108, which accesses data in the knowledge cloud 106, the natural language database 122, and/or the distributed ledger 118.

In various embodiments, the several computing devices executing within the cognitive intelligence platform are communicably coupled by way of a network/bus interface. Furthermore, the various components (e.g., the knowledge cloud 106, the critical thinking engine 108, the cognitive agent 110, and the node 116), are communicably coupled by one or more inter-host communication protocols 118. In one example, the knowledge cloud 106 is implemented using a first computing device, the critical thinking engine 108 is implemented using a second computing device, the cognitive agent 110 is implemented using a third computing device, and the node 116 is a fourth computing device, where each of the computing devices are coupled by way of the inter-host communication protocol 118. Although in this example, the individual components are described as executing on separate computing devices this example is not meant to be limiting, the components can be implemented on the same computing device, or partially on the same computing device, without departing from the scope of this disclosure.

The user device 104 represents any form of a computing device, or network of computing devices, e.g., a personal computing device, a smart phone, a tablet, a wearable computing device, a notebook computer, a media player device, and a desktop computing device. The user device 104 includes a processor, at least one memory, and at least one storage. A user uses the user device 104 to input a given text posed in natural language (e.g., typed on a physical keyboard, spoken into a microphone, typed on a touch screen, or combinations thereof) and interacts with the cognitive intelligence platform 102, by way of the cognitive agent 110.

A user (e.g., patient entity) may also use a software application installed on the user device 104 to request transactions to be performed using authentication information provided to the user device 104 during registration of the user as a node 116 on the blockchain network. Such an implementation makes the blockchain node 116 an active participant in the distributed ledger 118. In some embodiments, the transactions may request to access certain content stored on the distributed ledger 118. For example, a user may desire to view a care plan for diabetes. In some instances, the distributed ledger 118 can execute one or more rules to determine which care plan for diabetes was written by the most prestigious physician (e.g., based on peer and/or patient reviews), by physicians with medical degrees from certain medical schools, has been verified by the most or a threshold amount of physicians, has been viewed by the most or a threshold amount of physicians, is valid within a certain time period, and the like.

In some embodiments, the user may obtain content from a doctor's office, a public kiosk, a website, or the like and may desire to verify the source of the content and determine whether it is trustworthy. The user may use the software application to search for that particular content and the distributed ledger 118 may provide information to the user device 104 pertaining to the content, such as who the author of the content is, whether the author is associated with valid authorization information (e.g., medical license), whether the content has been verified by other medical personnel entities, how many times other medical personnel entities have viewed and/or used the content, and/or the like. Based on the information presented by the software application, the user may determine whether to trust and/or use the content.

The architecture 100 includes a network 120 that communicatively couples various devices, including the cognitive intelligence platform 102 and the user device 104. The network 120 can include local area network (LAN) and wide area networks (WAN). The network 102 can include wired technologies (e.g., Ethernet®) and wireless technologies (e.g., Wi-Fi®, code division multiple access (CDMA), global system for mobile (GSM), universal mobile telephone service (UMTS), Bluetooth®, and ZigBee®. For example, the user device 104 can use a wired connection or a wireless technology (e.g., Wi-Fi®) to transmit and receive data over the network 120.

Still referring to FIG. 1, the knowledge cloud 106 is configured to receive data from various sources and entities and integrate the data in a database. An example source that provides data to the knowledge could 106 is the service provider 112, an entity that provides a type of service to a user. For example, the service provider 112 can be a health service provider (e.g., a doctor's office, a physical therapist's office, a nurse's office, or a clinical social worker's office), and a financial service provider (e.g., an accountant's office). For purposes of this discussion, the cognitive intelligence platform 102 provides services in the health industry (e.g., a healthcare ecosystem), thus the examples discussed herein are associated with the health industry. However, any service industry can benefit from the disclosure herein, and thus the examples associated with the health industry are not meant to be limiting.

Throughout the course of a relationship between the service provider 112 and a user (e.g., the service provider 112 provides healthcare to a patient), the service provider 112 collects and generates data associated with the patient or the user, including health records that include doctor's notes and prescriptions, billing records, and insurance records. The service provider 112, using a computing device (e.g., a desktop computer or a tablet), provides the data associated with the user to the cognitive intelligence platform 102, and more specifically the knowledge cloud 106. This data associated with the user may be stored in the distributed ledger 118, in some embodiments.

In some embodiments, the service provider 112 may be logged into a software application on a computing device that communicates with the cognitive intelligence platform 102 via the cognitive agent 110. The service provider 112 may use the software application to make transaction requests to the distributed ledger 118 at a node 116 associated with the service provider 112. For example, a medical personnel entity (e.g., service provider 112) may use the software application to request to store content at the distributed ledger 118. The content may pertain to healthcare and may include a new portion of knowledge/information that the medical personnel entity discovered or decided to include in a care plan. In one example, the content may include a self-care component to a diabetes care plan that the medical personnel entity determined provides better outcomes for patients.

When the transmission request is received, the distributed ledger 118 may use one or more rules to determine whether the service provider 112 has proper authentication information for a registered node 116 on the blockchain network. The distributed ledger 118 may use one or more rules to determine whether the service provider 112 is associated with valid authorization information on the distributed ledger 118. The distributed ledger 118 may use one or more rules to determine whether at least a portion of the content provided by the service provider 112 is new relative to other content stored on the distributed ledger 118. For example, the distributed ledger 118 may analyze other pieces of content related to diabetes care plans that are stored on the distributed ledger 118 to determine if any of them disclose the particular methodology of self-care added to the submitted content by the service provider 112. In some embodiments, the content submitted and the other content stored on the distributed ledger 118 may be parsed and compared (e.g., string comparisons) to determine if there is matching text. If a threshold amount of the content matches between the submitted content and the other content stored on the distributed ledger 118, the one or more rules may determine that the submitted content is not new. If each of the applicable one or more rules are satisfied and/or a consensus of nodes 116 approve the transaction, then the content may be stored on the distributed ledger 118. If any of the one or more rules described above are not satisfied and/or the consensus of nodes 116 do not approve the transaction, then the submitted content may not be stored on the distributed ledger 118.

In another example, a medical personnel entity (e.g., service provider 112) may use the software application to request to view and/or verify content stored on the distributed ledger 118. When the transmission request is received, the distributed ledger 118 may use one or more rules to determine whether the service provider 112 has proper authentication information for a registered node 116 on the blockchain network. The distributed ledger 118 may use one or more rules to determine whether the service provider 112 has a proper access right to access the content and/or whether the service provider 112 is associated with valid authorization information on the distributed ledger 118. In some embodiments, the content may be set to private and the service provider 112 may purchase a right to access the content. In other instances, where the service provider 112 is a part of a same organization as the author of the content, the service provider 112 may be granted the access right.

A computing device associated with the service provider 112 may be provided with the requested content. The distributed ledger 118 may be updated to reflect that the distributed ledger 118 has been viewed by the service provider 118. The service provider 112 may review the content and transmit, via a computing device, a transaction request to the distributed ledger 118 where the transaction request includes an operation to verify the content. Verifying the content may include the distributed ledger 118 using one or more rules to determine that the service provider 112 is associated with valid authorization information. If the one or more rules are satisfied and/or a consensus of nodes 116 approve the transaction, the distributed ledger 112 may update a record to indicate that this particular content has been verified by another service provider 112 besides the author/creator of the content.

In some embodiments, once the content is received by the computing device of the requesting service provider 112, the service provider 112 may modify the content, by adding additional content (e.g., a video of additional steps) not disclosed in the original content, and transmit a transaction request to the distributed ledger 118 to store the updated content including the additional content (video) and the original content. The distributed ledger 118 may use one or more rules to determine whether the service provider 112 is associated with valid authorization information and/or whether the updated content includes new content that is not disclosed by other content in the distributed ledger 118. If the one or more rules and/or the consensus protocol are satisfied, the updated content may be stored on the distributed ledger 118.

Another example source that provides data to the knowledge cloud 106 is the facility 114 (e.g., medical facility entity). The facility 114 represents a location owned, operated, or associated with any entity including the service provider 112. As used herein, an entity represents an individual or a collective with a distinct and independent existence. An entity can be legally recognized (e.g., a sole proprietorship, a partnership, a corporation) or less formally recognized in a community. For example, the entity can include a company that owns or operates a gym (facility). Additional examples of the facility 114 include, but is not limited to, a hospital, a trauma center, a clinic, a dentist's office, a pharmacy, a store (including brick and mortar stores and online retailers), an out-patient care center, a specialized care center, a birthing center, a gym, a cafeteria, and a psychiatric care center.

As the facility 114 represents a large number of types of locations, for purposes of this discussion and to orient the reader by way of example, the facility 114 represents the doctor's office or a gym. The facility 114 generates additional data associated with the user such as appointment times, an attendance record (e.g., how often the user goes to the gym), a medical record, a billing record, a purchase record, an order history, and an insurance record. The facility 114, using a computing device (e.g., a desktop computer or a tablet), provides the data associated with the user to the cognitive intelligence platform 102, and more specifically the knowledge cloud 106. This data associated with the user may be stored in the distributed ledger 118, in some embodiments.

For example, the facility 114 may use a computing device associated with the facility to make requests for transactions to be performed using authentication information provided to the computing device during registration of the facility 114 as a node 116 on the blockchain network. In some embodiments, the transactions may include requesting storage, on the distributed ledger 118, of evidence-based guidelines that are generated as a result of clinical trials or studies performed by medical personnel entities of the facility 114, and/or of results of the clinical trials, studies, and/or the like on the distributed ledger 118. The facility 114 may also transmit, using the computing device, a transaction request to view content stored on the distributed ledger 118. For example, when a patient is consulting a physician at the facility 114, the facility may request information pertaining to a care plan to provide to the patient. The information may be transmitted to a computing device of the patient (e.g., user device 104) and the user may use the software application to verify the source of the content, when the content was generated, whether the source/creator of the content is associated with valid authorization information, whether the content has been verified within a certain time frame, and/or the like.

An additional example source that provides data to the knowledge cloud 106 is the microsurvey 116. The microsurvey 116 represents a tool created by the cognitive intelligence platform 102 that enables the knowledge cloud 106 to collect additional data associated with the user. The microsurvey 116 is originally provided by the cognitive intelligence platform 102 (by way of the cognitive agent 110) and the user provides data responsive to the microsurvey 116 using the user device 104. Additional details of the microsurvey 116 are described below.

Yet another example source that provides data to the knowledge cloud 106, is the cognitive intelligence platform 102, itself. In order to address the care needs and well-being of the user, the cognitive intelligence platform 102 collects, analyzes, and processes information from the user, healthcare providers, and other eco-system participants, and consolidates and integrates the information into knowledge. The knowledge can be shared with the user and stored in the knowledge cloud 106.

In various embodiments, the computing devices used by the service provider 112 and the facility 114 are communicatively coupled to the cognitive intelligence platform 102, by way of the network 120. While data is used individually by various entities including: a hospital, practice group, facility, or provider, the data is less frequently integrated and seamlessly shared between the various entities in the current art. The cognitive intelligence platform 102 provides a solution that integrates data from the various entities. That is, the cognitive intelligence platform 102 ingests, processes, and disseminates data and knowledge in an accessible fashion, where the reason for a particular answer or dissemination of data is accessible by a user.

In particular, the cognitive intelligence platform 102 (e.g., by way of the cognitive agent 110 interacting with the user) holistically manages and executes a health plan for durational care and wellness of the user (e.g., a patient or consumer). The health plan includes various aspects of durational management that is coordinated through a care continuum.

The cognitive agent 110 can implement various personas that are customizable. For example, the personas can include knowledgeable (sage), advocate (coach), and witty friend (jester). And in various embodiments, the cognitive agent 110 persists with a user across various interactions (e.g., conversations streams), instead of being transactional or transient. Thus, the cognitive agent 110 engages in dynamic conversations with the user, where the cognitive intelligence platform 102 continuously deciphers topics that a user wants to talk about. The cognitive intelligence platform 102 has relevant conversations with the user by ascertaining topics of interest from a given text posed in a natural language input by the user. Additionally the cognitive agent 110 connects the user to healthcare service providers, hyperlocal health communities, and a variety of services and tools/devices, based on an assessed interest of the user. In some embodiments, the cognitive agent 110 may connect the user to healthcare service providers that are in a vicinity of the geolocation of the user device 104 based on certain information stored in the distributed ledger 118 (e.g., which healthcare service providers have valid authorization information, location of the healthcare service providers, specialty of the healthcare service provider, health issue of the patient, etc.).

As the cognitive agent 110 persists with the user, the cognitive agent 110 can also act as a coach and advocate while delivering pieces of information to the user based on tonal knowledge, human-like empathies, and motivational dialog within a respective conversational stream, where the conversational stream is a technical discussion focused on a specific topic. Overall, in response to a question—e.g., posed by the user in natural language—the cognitive intelligence platform 102 consumes data from and related to the user and computes an answer. The answer is generated using a rationale that makes use of common sense knowledge, domain knowledge, evidence-based medicine guidelines, clinical ontologies, and curated medical advice. Thus, the content displayed by the cognitive intelligence platform 102 (by way of the cognitive agent 110) is customized based on the language used to communicate with the user, as well as factors such as a tone, goal, and depth of topic to be discussed.

Overall, the cognitive intelligence platform 102 may be accessible to a user (e.g., patient entity), medical facility entities (e.g., a hospital system, clinics, pharmacies, etc.), medical personnel entities (e.g., physicians, pharmacists, dentists, optometrists, etc.), insurance provider entities, professional association entities, and government agency entities. Additionally, the cognitive intelligence platform 102 is accessible to paying entities interested in user behavior—e.g., the outcome of physician-consumer interactions in the context of disease or the progress of risk management. Additionally, entities that provides specialized services such as tests, therapies, and clinical processes that need risk based interactions can also receive filtered leads from the cognitive intelligence platform 102 for potential clients.

Conversational Analysis

In various embodiments, the cognitive intelligence platform 102 is configured to perform conversational analysis in a general setting. The topics covered in the general setting is driven by the combination of agents (e.g., cognitive agent 110) selected by a user. In some embodiments, the cognitive intelligence platform 102 uses conversational analysis to identify the intent of the user (e.g., find data, ask a question, search for facts, find references, and find products) and a respective micro-theory in which the intent is logical.

For example, the cognitive intelligence platform 102 applies conversational analysis to decode what the user is asking or stated, where the question or statement is in free form language (e.g., natural language). Prior to determining and sharing knowledge (e.g., with the user or the knowledge cloud 106), using conversational analysis, the cognitive intelligence platform 102 identifies an intent of the user and overall conversational focus.

The cognitive intelligence platform 102 responds to a statement or question according to the conversational focus and steers away from another detected conversational focus so as to focus on a goal defined by the cognitive agent 110. Given an example statement of a user, “I want to fly out tomorrow,” the cognitive intelligence platform 102 uses conversational analysis to determine an intent of the statement. Is the user aspiring to be bird-like or does he want to travel? In the former case, the micro-theory is that of human emotions whereas in the latter case, the micro-theory is the world of travel. Answers are provided to the statement depending on the micro-theory in which the intent logically falls.

The cognitive intelligence platform 102 utilizes a combination of linguistics, artificial intelligence, and decision trees to decode what a user is asking or stating. The discussion includes methods and system design considerations and results from an existing embodiment. Additional details related to conversational analysis are discussed next.

Analyzing Conversational Context As Part of Conversational Analysis

For purposes of this discussion, the concept of analyzing conversational context as part of conversational analysis is now described. To analyze conversational context, the following steps are taken: 1) obtain text (e.g., receive a question) and perform translations; 2) understand concepts, entities, intents, and micro-theory; 3) relate and search; 4) ascertain the existence of related concepts; 5) logically frame concepts or needs; 6) understand the questions that can be answered from available data; and 7) answer the question. Each of the foregoing steps is discussed next, in turn.

Step 1: Obtain Text/Question and Perform Translations

In various embodiments, the cognitive intelligence platform 102 (FIG. 1) receives a text or question and performs translations as appropriate. The cognitive intelligence platform 102 supports various methods of input including text received from a touch interface (e.g., options presented in a microsurvey), text input through a microphone (e.g., words spoken into the user device), and text typed on a keyboard or on a graphical user interface. Additionally, the cognitive intelligence platform 102 supports multiple languages and auto translation (e.g., from English to Traditional/Simplified Chinese or vice versa).

The example text below is used to described methods in accordance with various embodiments herein:
“One day in January 1913. G.H. Hardy, a famous Cambridge University mathematician received a letter from an Indian named Srinivasa Ramanuj an asking him for his opinion of 120 mathematical theorems that Ramanujan said he had discovered. To Hardy, many of the theorems made no sense. Of the others, one or two were already well-known. Ramanujan must be some kind of trickplayer, Hardy decided, and put the letter aside. But all that day the letter kept hanging round Hardy. Might there by something in those wild-looking theorems?
That evening Hardy invited another brilliant Cambridge mathematician, J. E. Littlewood, and the two men set out to assess the Indian's worth. That incident was a turning point in the history of mathematics.
At the time, Ramanujan was an obscure Madras Port Trust clerk. A little more than a year later, he was at Cambridge University, and beginning to be recognized as one of the most amazing mathematicians the world has ever known. Though he died in 1920, much of his work was so far in advance of his time that only in recent years is it beginning to be properly understood.
Indeed, his results are helping solve today's problems in computer science and physics, problems that he could have had no notion of.
For Indians, moreover, Ramanujan has a special significance. Ramanujan, through born in poor and ill-paid accountant's family 100 years ago, has inspired many Indians to adopt mathematics as career.
Much of Ramanujan's work is in number theory, a branch of mathematics that deals with the subtle laws and relationships that govern numbers. Mathematicians describe his results as elegant and beautiful but they are much too complex to be appreciated by laymen.
His life, though, is full of drama and sorrow. It is one of the great romantic stories of mathematics, a distressing reminder that genius can surface and rise in the most unpromising circumstances.”

The cognitive intelligence platform 102 analyzes the example text above to detect structural elements within the example text (e.g., paragraphs, sentences, and phrases). In some embodiments, the example text is compared to other sources of text such as dictionaries, and other general fact databases (e.g., Wikipedia) to detect synonyms and common phrases present within the example text.

Step 2: Understand Concept, Entity, Intent, and Micro-Theory

In step 2, the cognitive intelligence platform 102 parses the text to ascertain concepts, entities, intents, and micro-theories. An example output after the cognitive intelligence platform 102 initially parses the text is shown below, where concepts, and entities are shown in bold.
“One day in January 1913. G.H. Hardy, a famous Cambridge University mathematician received a letter from an Indian named Srinivasa Ramanujan asking him for his opinion of 120 mathematical theorems that Ramanujan said he had discovered. To Hardy, many of the theorems made no sense. Of the others, one or two were already well-known. Ramanujan must be some kind of trickplayer, Hardy decided, and put the letter aside. But all that day the letter kept hanging round Hardy. Might there by something in those wild-looking theorems?
That evening Hardy invited another brilliant Cambridge mathematician, J. E. Littlewood, and the two men set out to assess the Indian's worth. That incident was a turning point in the history of mathematics.
At the time, Ramanujan was an obscure Madras Port Trust clerk. A little more than a year later, he was at Cambridge University, and beginning to be recognized as one of the most amazing mathematicians the world has ever known.
Though he died in 1920, much of his work was so far in advance of his time that only in recent years is it beginning to be properly understood.
Indeed, his results are helping solve today's problems in computer science and physics, problems that he could have had no notion of.
For Indians, moreover, Ramanujan has a special significance. Ramanujan, through born in poor and ill-paid accountant's family 100 years ago, has inspired many Indians to adopt mathematics as career.
Much of Ramanujan's work is in number theory, a branch of mathematics that deals with the subtle laws and relationships that govern numbers. Mathematicians describe his results as elegant and beautiful but they are much too complex to be appreciated by laymen.
His life, though, is full of drama and sorrow. It is one of the great romantic stories of mathematics, a distressing reminder that genius can surface and rise in the most unpromising circumstances.”

For example, the cognitive intelligence platform 102 ascertains that Cambridge is a university—which is a full understanding of the concept. The cognitive intelligence platform (e.g., the cognitive agent 110) understands what humans do in Cambridge, and an example is described below in which the cognitive intelligence platform 102 performs steps to understand a concept.

For example, in the context of the above example, the cognitive agent 110 understands the following concepts and relationships:

Cambridge employed John Edensor Littlewood (1)

Cambridge has the position Ramanujan's position at Cambridge University (2)

Cambridge employed G. H. Hardy. (3)

The cognitive agent 110 also assimilates other understandings to enhance the concepts, such as:

Cambridge has Trinity College as a suborganization. (4)

Cambridge is located in Cambridge. (5)

Alan Turing is previously enrolled at Cambridge. (6)

Stephen Hawking attended Cambridge. (7)

The statements (1)-(7) are not picked at random. Instead the cognitive agent 110 dynamically constructs the statements (1)-(7) from logic or logical inferences based on the example text above. Formally, the example statements (1)-(7) are captured as follows:

(#$subOrganizations #$UniversityOfCambridge #$TrinityCollege-Cambridge-England) (8)

(#$placeInCity #$UniversityOfCambridge #$Cityof CambridgeEngland) (9)

(#$schooling #$AlanTuring #$UniversityOfCambridge #$PreviouslyEnrolled) (10)

(#$hasAlumni #$UniversityOfCambridge #$StephenHawking) (11)

Step 3: Relate and Search

Next, in step 3, the cognitive agent 110 relates various entities and topics and follows the progression of topics in the example text. Relating includes the cognitive agent 110 understanding the different instances of Hardy are all the same person, and the instances of Hardy are different from the instances of Littlewood. The cognitive agent 110 also understands that the instances Hardy and Littlewood share some similarities—e.g., both are mathematicians and they did some work together at Cambridge on Number Theory. The ability to track this across the example text is referred to as following the topic progression with a context.

Step 4: Ascertain the Existence of Related Concepts

Next, in Step 4, the cognitive agent 110 asserts non-existent concepts or relations to form new knowledge. Step 4 is an optional step for analyzing conversational context. Step 4 enhances the degree to which relationships are understood or different parts of the example text are understood together. If two concepts appear to be separate—e.g., a relationship cannot be graphically drawn or logically expressed between enough sets of concepts—there is a barrier to understanding. The barriers are overcome by expressing additional relationships. The additional relationships can be discovered using strategies like adding common sense or general knowledge sources (e.g., using the common sense data 208) or adding in other sources including a lexical variant database, a dictionary, and a thesaurus.

One example of concept progression from the example text is as follows: the cognitive agent 110 ascertains the phrase “theorems that Ramanujan said he had discovered” is related to the phrase “his results”, which is related to “Ramanujan's work is in number theory, a branch of mathematics that deals with the subtle laws and relationships that govern numbers.”

Step 5: Logically Frame Concepts or Needs

In Step 5, the cognitive agent 110 determines missing parameters—which can include for example, missing entities, missing elements, and missing nodes—in the logical framework (e.g., with a respective micro-theory). The cognitive agent 110 determines sources of data that can inform the missing parameters. Step 5 can also include the cognitive agent 110 adding common sense reasoning and finding logical paths to solutions.

With regards to the example text, some common sense concepts include:

Mathematicians develop Theorems. (12)

Theorems are hard to comprehend. (13)

Interpretations are not apparent for years. (14)

Applications are developed over time. (15)

Mathematicians collaborate and assess work. (16)

With regards to the example text, some passage concepts include:

Ramanujan did Theorems in Early 20^{th} Century. (17)

Hardy assessed Ramanujan's Theorems. (18)

Hardy collaborated with Littlewood. (19)

Hardy and Littlewood assessed Ramanujan's work (20)

Within the micro-theory of the passage analysis, the cognitive agent 110 understands and catalogs available paths to answer questions. In Step 5, the cognitive agent 110 makes the case that the concepts (12)-(20) are expressed together.

Step 6: Understand the Questions that can be Answered from Available Data

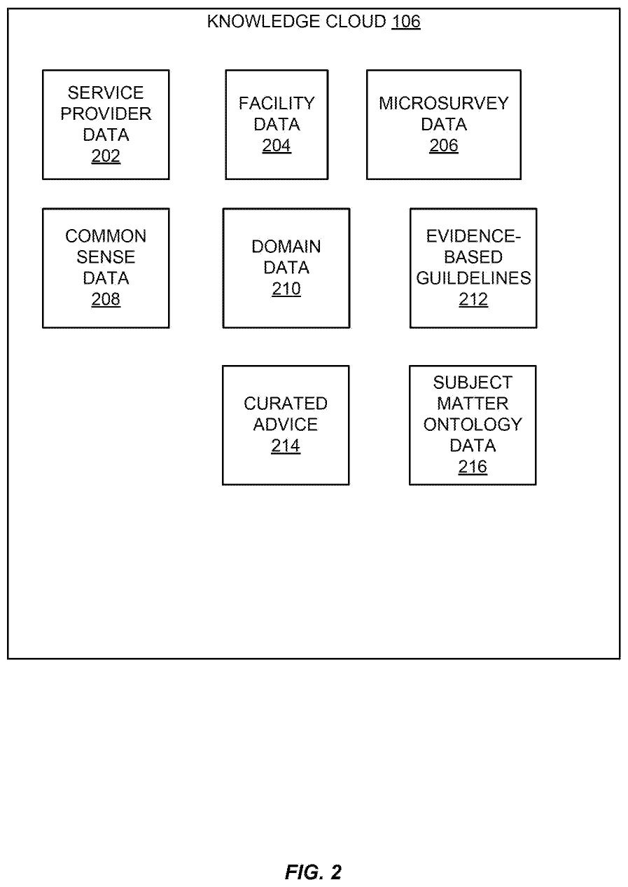

In Step 6, the cognitive agent 110 parses sub-intents and entities. Given the example text, the following questions are answerable from the cognitive agent's developed understanding of the example text, where the understanding was developed using information and context ascertained from the example text as well as the common sense data 208 (FIG. 2):

What situation causally contributed to Ramanujan's position at Cambridge? (21)

Does the author of the passage regret that Ramanujan died prematurely? (22)

Does the author of the passage believe that Ramanujan is a mathematical genius? (23)

Based on the information that is understood by the cognitive agent 110, the questions (21)-(23) can be answered.

By using an exploration method such as random walks, the cognitive agent 110 makes a determination as the paths that are plausible and reachable with the context (e.g., micro-theory) of the example text. Upon explorations, the cognitive agent 110 catalogs a set of meaningful questions. The set of meaningful questions are not asked, but instead explored based on the cognitive agent's understanding of the example text.

Given the example text, an example of exploration that yields a positive result is: “a situation X that caused Ramanujan's position.” In contrast, an example of exploration that causes irrelevant results is: “a situation Y that caused Cambridge.” The cognitive agent 110 is able to deduce that the latter exploration is meaningless, in the context of a micro-theory, because situations do not cause universities. Thus the cognitive agent 110 is able to deduce, there are no answers to Y, but there are answers to X.

Step 7: Answer the Question

In Step 7, the cognitive agent 110 provides a precise answer to a question. For an example question such as: “What situation causally contributed to Ramanujan's position at Cambridge?” the cognitive agent 110 generates a precise answer using the example reasoning:

HardyandLittlewoodsEvaluatingOfRamanujansWork (24)

HardyBeliefThatRamanujanIsAnExpertInMathematics (25)

HardysBeliefThatRamanujanIsAnExpertInMathematicsAndAGenius (26)

In order to generate the above reasoning statements (24)-(26), the cognitive agent 110 utilizes a solver or prover in the context of the example text's micro-theory—and associated facts, logical entities, relations, and assertions. As an additional example, the cognitive agent 110 uses a reasoning library that is optimized for drawing the example conclusions above within the fact, knowledge, and inference space (e.g., work space) that the cognitive agent 110 maintains.

By implementing the steps 1-7, the cognitive agent 110 analyzes conversational context. The described method for analyzing conversation context can also be used for recommending items in conversations streams. A conversational stream is defined herein as a technical discussion focused on specific topics. As related to described examples herein, the specific topics relate to health (e.g., diabetes). Throughout the lifetime of a conversational stream, a cognitive agent 110 collect information over may channels such as chat, voice, specialized applications, web browsers, contact centers, and the like.

By implementing the methods to analyze conversational context, the cognitive agent 110 can recommend a variety of topics and items throughout the lifetime of the conversational stream. Examples of items that can be recommended by the cognitive agent 110 include: surveys, topics of interest, local events, devices or gadgets, dynamically adapted health assessments, nutritional tips, reminders from a health events calendar, and the like.

Accordingly, the cognitive intelligence platform 102 provides a platform that codifies and takes into consideration a set of allowed actions and a set of desired outcomes. The cognitive intelligence platform 102 relates actions, the sequences of subsequent actions (and reactions), desired sub-outcomes, and outcomes, in a way that is transparent and logical (e.g., explainable). The cognitive intelligence platform 102 can plot a next best action sequence and a planning basis (e.g., health care plan template, or a financial goal achievement template), also in a manner that is explainable. The cognitive intelligence platform 102 can utilize a critical thinking engine 108 and a natural language database 122 (e.g., a linguistics and natural language understanding system) to relate conversation material to actions.

For purposes of this discussion, several examples are discussed in which conversational analysis is applied within the field of durational and whole-health management for a user. The discussed embodiments holistically address the care needs and well-being of the user during the course of his life. The methods and systems described herein can also be used in fields outside of whole-health management, including: phone companies that benefits from a cognitive agent; hospital systems or physicians groups that want to coach and educate patients; entities interested in user behavior and the outcome of physician-consumer interactions in terms of a progress of disease or risk management; entities that provide specialized services (e.g., test, therapies, clinical processes) to filter leads; and sellers, merchants, stores and big box retailers that want to understand which product to sell.

FIG. 2 shows additional details of a knowledge cloud, in accordance with various embodiments. In particular, FIG. 2 illustrates various types of data received from various sources, including service provider data 202, facility data 204, microsurvey data 206, commonsense data 208, domain data 210, evidence-based guidelines 212, subject matter ontology data 214, and curated advice 216. The types of data represented by the service provider data 202 and the facility data 204 include any type of data generated by the service provider 112 and the facility 114, and the above examples are not meant to be limiting. Thus, the example types of data are not meant to be limiting and other types of data can also be stored within the knowledge cloud 106 without departing from the scope of this disclosure.

The service provider data 202 is data provided by the service provider 112 (described in FIG. 1) and the facility data 204 is data provided by the facility 114 (described in FIG. 1). For example, the service provider data 202 includes medical records of a respective patient of a service provider 112 that is a doctor. In another example, the facility data 204 includes an attendance record of the respective patient, where the facility 114 is a gym. The microsurvey data 206 is data provided by the user device 104 responsive to questions presented in the microsurvey 116 (FIG. 1).

Common sense data 208 is data that has been identified as “common sense”, and can include rules that govern a respective concept and used as glue to understand other concepts.

Domain data 210 is data that is specific to a certain domain or subject area. The source of the domain data 210 can include digital libraries. In the healthcare industry, for example, the domain data 210 can include data specific to the various specialties within healthcare such as, obstetrics, anesthesiology, and dermatology, to name a few examples. In the example described herein, the evidence-based guidelines 212 include systematically developed statements to assist practitioner and patient decisions about appropriate health care for specific clinical circumstances.

Curated advice 214 includes advice from experts in a subject matter. The curated advice 214 can include peer-reviewed subject matter, and expert opinions. Subject matter ontology data 216 includes a set of concepts and categories in a subject matter or domain, where the set of concepts and categories capture properties and relationships between the concepts and categories.

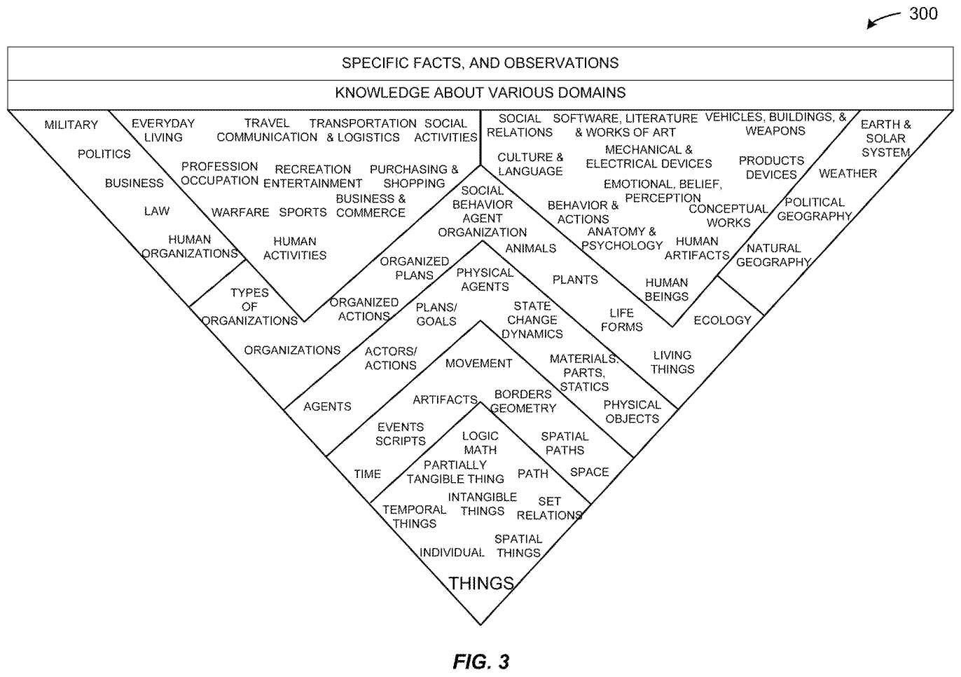

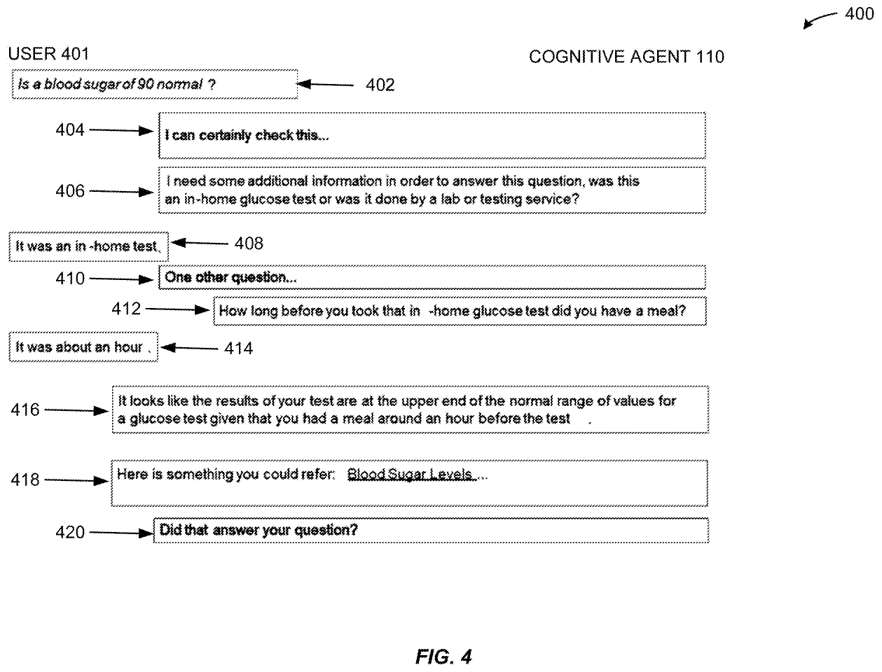

FIG. 3 illustrates an example subject matter ontology 300 that is included as part of the subject matter ontology data 216. FIG. 4 illustrates aspects of a conversation 400 between a user and the cognitive intelligence platform 102, and more specifically the cognitive agent 110. For purposes of this discussion, the user 401 is a patient of the service provider 112. The user interacts with the cognitive agent 110 using a computing device, a smart phone, or any other device configured to communicate with the cognitive agent 110 (e.g., the user device 104 in FIG. 1). The user can enter text into the device using any known means of input including a keyboard, a touchscreen, and a microphone. The conversation 400 represents an example graphical user interface (GUI) presented to the user 401 on a screen of his computing device.

Initially, the user asks a general question, which is treated by the cognitive agent 110 as an “originating question.” The originating question is classified into any number of potential questions (“pursuable questions”) that are pursued during the course of a subsequent conversation. In some embodiments, the pursuable questions are identified based on a subject matter domain or goal. In some embodiments, classification techniques are used to analyze language (e.g., such as those outlined in HPS ID20180901-01_method for conversational analysis). Any known text classification technique can be used to analyze language and the originating question. For example, in line 402, the user enters an originating question about a subject matter (e.g., blood sugar) such as: “Is a blood sugar of 90 normal” ? I

In response to receiving an originating question, the cognitive intelligence platform 102 (e.g., the cognitive agent 110 operating in conjunction with the critical thinking engine 108) performs a first round of analysis (e.g., which includes conversational analysis) of the originating question and, in response to the first round of analysis, creates a workspace and determines a first set of follow up questions.

In various embodiments, the cognitive agent 110 may go through several rounds of analysis executing within the workspace, where a round of analysis includes: identifying parameters, retrieving answers, and consolidating the answers. The created workspace can represent a space where the cognitive agent 110 gathers data and information during the processes of answering the originating question. In various embodiments, each originating question corresponds to a respective workspace. The conversation orchestrator 124 can assess data present within the workspace and query the cognitive agent 110 to determine if additional data or analysis should be performed.

In particular, the first round of analysis is performed at different levels, including analyzing natural language of the text, and analyzing what specifically is being asked about the subject matter (e.g., analyzing conversational context). The first round of analysis is not based solely on a subject matter category within which the originating question is classified. For example, the cognitive intelligence platform 102 does not simply retrieve a predefined list of questions in response to a question that falls within a particular subject matter, e.g., blood sugar. That is, the cognitive intelligence platform 102 does not provide the same list of questions for all questions related to the particular subject matter. Instead, for example, the cognitive intelligence platform 102 creates dynamically formulated questions, curated based on the first round of analysis of the originating question.

In particular, during the first round of analysis, the cognitive agent 110 parses aspects of the originating question into associated parameters. The parameters represent variables useful for answering the originating question. For example, the question “is a blood sugar of 90 normal” may be parsed and associated parameters may include, an age of the inquirer, the source of the value 90 (e.g., in home test or a clinical test), a weight of the inquirer, and a digestive state of the user when the test was taken (e.g., fasting or recently eaten). The parameters identify possible variables that can impact, inform, or direct an answer to the originating question.

For purposes of the example illustrated in FIG. 4, in the first round of analysis, the cognitive intelligence platform 102 inserts each parameter into the workspace associated with the originating question (line 402). Additionally, based on the identified parameters, the cognitive intelligence platform 102 identifies a customized set of follow up questions (“a first set of follow-up questions). The cognitive intelligence platform 102 inserts first set of follow-up questions in the workspace associated with the originating question.

The follow up questions are based on the identified parameters, which in turn are based on the specifics of the originating question (e.g., related to an identified micro-theory). Thus the first set of follow-up questions identified in response to, if a blood sugar is normal, will be different from a second set of follow up questions identified in response to a question about how to maintain a steady blood sugar.

After identifying the first set of follow up questions, in this example first round of analysis, the cognitive intelligence platform 102 determines which follow up question can be answered using available data and which follow-up question to present to the user. As described over the next few paragraphs, eventually, the first set of follow-up questions is reduced to a subset (“a second set of follow-up questions”) that includes the follow-up questions to present to the user.

In various embodiments, available data is sourced from various locations, including a user account, the knowledge cloud 106, and other sources. Other sources can include a service that supplies identifying information of the user, where the information can include demographics or other characteristics of the user (e.g., a medical condition, a lifestyle). For example, the service can include a doctor's office or a physical therapist's office.

Another example of available data includes the user account. For example, the cognitive intelligence platform 102 determines if the user asking the originating question, is identified. A user can be identified if the user is logged into an account associated with the cognitive intelligence platform 102. User information from the account is a source of available data. The available data is inserted into the workspace of the cognitive agent 110 as a first data.

Another example of available data includes the data stored within the knowledge cloud 106. For example, the available data includes the service provider data 202 (FIG. 2), the facility data 204, the microsurvey data 206, the common sense data 208, the domain data 210, the evidence-based guidelines 212, the curated advice 214, and the subject matter ontology data 216. Additionally data stored within the knowledge cloud 106 includes data generated by the cognitive intelligence platform 102, itself.

Follow up questions presented to the user (the second set of follow-up questions) are asked using natural language and are specifically formulated (“dynamically formulated question”) to elicit a response that will inform or fulfill an identified parameter. Each dynamically formulated question can target one parameter at a time. When answers are received from the user in response to a dynamically formulated question, the cognitive intelligence platform 102 inserts the answer into the workspace. In some embodiments, each of the answers received from the user and in response to a dynamically formulated question, is stored in a list of facts. Thus the list of facts include information specifically received from the user, and the list of facts is referred to herein as the second data.

With regards to the second set of follow-up questions (or any set of follow-up questions), the cognitive intelligence platform 102 calculates a relevance index, where the relevance index provides a ranking of the questions in the second set of follow-up questions. The ranking provides values indicative of how relevant a respective follow-up question is to the originating question. To calculate the relevance index, the cognitive intelligence platform 102 can use conversations analysis techniques described in HPS ID20180901-01_method. In some embodiments, the first set or second set of follow up questions is presented to the user in the form of the microsurvey 116.

In this first round of analysis, the cognitive intelligence platform 102 consolidates the first and second data in the workspace and determines if additional parameters need to be identified, or if sufficient information is present in the workspace to answer the originating question. In some embodiments, the cognitive agent 110 (FIG. 1) assesses the data in the workspace and queries the cognitive agent 110 to determine if the cognitive agent 110 needs more data in order to answer the originating question. The conversation orchestrator 124 executes as an interface

For a complex originating question, the cognitive intelligence platform 102 can go through several rounds of analysis. For example, in a first round of analysis the cognitive intelligence platform 102 parses the originating question. In a subsequent round of analysis, the cognitive intelligence platform 102 can create a sub question, which is subsequently parsed into parameters in the subsequent round of analysis. The cognitive intelligence platform 102 is smart enough to figure out when all information is present to answer an originating question without explicitly programming or pre-programming the sequence of parameters that need to be asked about.

In some embodiments, the cognitive agent 110 is configured to process two or more conflicting pieces of information or streams of logic. That is, the cognitive agent 110, for a given originating question can create a first chain of logic and a second chain of logic that leads to different answers. The cognitive agent 110 has the capability to assess each chain of logic and provide only one answer. That is, the cognitive agent 110 has the ability to process conflicting information received during a round of analysis.

Additionally, at any given time, the cognitive agent 110 has the ability to share its reasoning (chain of logic) to the user. If the user does not agree with an aspect of the reasoning, the user can provide that feedback which results in affecting change in a way the critical thinking engine 108 analyzed future questions and problems.

Subsequent to determining enough information is present in the workspace to answer the originating question, the cognitive agent 110 answers the question, and additionally can suggest a recommendation or a recommendation (e.g., line 418). The cognitive agent 110 suggests the reference or the recommendation based on the context and questions being discussed in the conversation (e.g., conversation 400). The reference or recommendation serves as additional handout material to the user and is provided for informational purposes. The reference or recommendation often educates the user about the overall topic related to the originating question.

In the example illustrated in FIG. 4, in response to receiving the originating questions (line 402), the cognitive intelligence platform 102 (e.g., the cognitive agent 110 in conjunction with the critical thinking engine 108) parses the originating question to determine at least one parameter: location. The cognitive intelligence platform 102 categorizes this parameter, and a corresponding dynamically formulated question in the second set of follow-up questions. Accordingly, in lines 404 and 406, the cognitive agent 110 responds by notifying the user “I can certainly check this . . . ” and asking the dynamically formulated question “I need some additional information in order to answer this question, was this an in-home glucose test or was it done by a lab or testing service?”

The user 401 enters his answer in line 408: “It was an in-home test,” which the cognitive agent 110 further analyzes to determine additional parameters: e.g., a digestive state, where the additional parameter and a corresponding dynamically formulated question as an additional second set of follow-up questions. Accordingly, the cognitive agent 110 poses the additional dynamically formulated question in lines 410 and 412: “One other question . . . ” and “How long before you took that in-home glucose test did you have a meal?” The user provides additional information in response “it was about an hour” (line 414).

The cognitive agent 110 consolidates all the received responses using the critical thinking engine 108 and the knowledge cloud 106 and determines an answer to the initial question posed in line 402 and proceeds to follow up with a final question to verify the user's initial question was answered. For example, in line 416, the cognitive agent 110 responds: “It looks like the results of your test are at the upper end of the normal range of values for a glucose test given that you had a meal around an hour before the test.” The cognitive agent 110 provides additional information (e.g., provided as a link): “Here is something you could refer,” (line 418), and follows up with a question “Did that answer your question?” (line 420).

As described above, due to the natural language database 108, in various embodiments, the cognitive agent 110 is able to analyze and respond to questions and statements made by a user 401 in natural language. That is, the user 401 is not restricted to using certain phrases in order for the cognitive agent 110 to understand what a user 401 is saying. Any phrasing, similar to how the user would speak naturally can be input by the user and the cognitive agent 110 has the ability to understand the user.

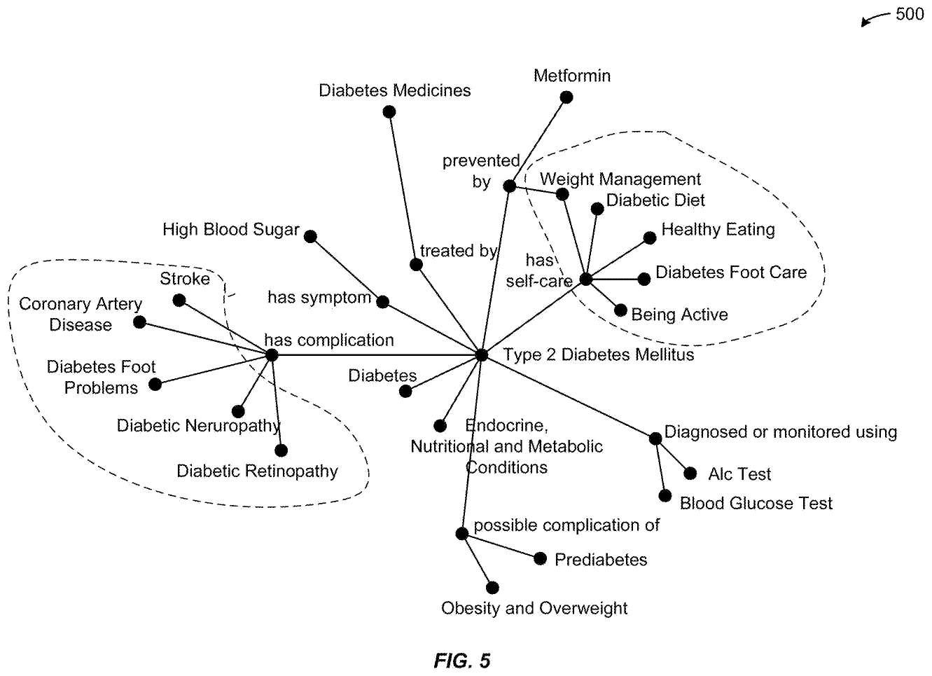

FIG. 5 illustrates a cognitive map or “knowledge graph” 500, in accordance with various embodiments. In particular, the knowledge graph represents a graph traversed by the cognitive intelligence platform 102, when assessing questions from a user with Type 2 diabetes. Individual nodes in the knowledge graph 500 represent a health artifact or relationship that is gleaned from direct interrogation or indirect interactions with the user (by way of the user device 104).

In one embodiment, the cognitive intelligence platform 102 identified parameters for an originating question based on a knowledge graph illustrated in FIG. 5. For example, the cognitive intelligence platform 102 parses the originating question to determine which parameters are present for the originating question. In some embodiments, the cognitive intelligence platform 102 infers the logical structure of the parameters by traversing the knowledge graph 500, and additionally, knowing the logical structure enables the cognitive agent 110 to formulate an explanation as to why the cognitive agent 110 is asking a particular dynamically formulated question.

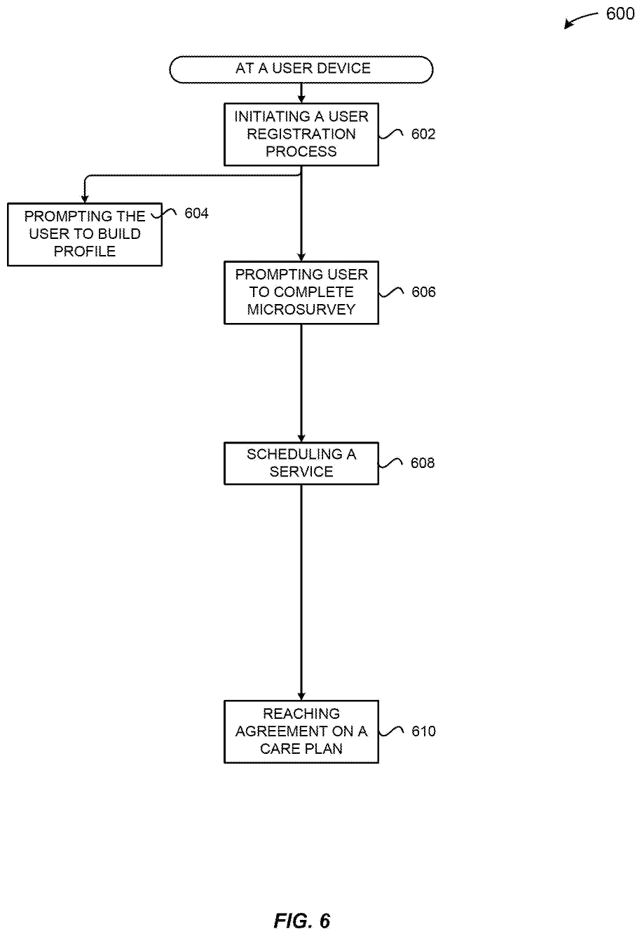

FIG. 6 shows a method, in accordance with various embodiments. The method is performed at a user device (e.g., the user device 102) and in particular, the method is performed by an application executing on the user device 102. The method begins with initiating a user registration process (block 602). The user registration can include tasks such as displaying a GUI asking the user to enter in personal information such as his name and contact information.

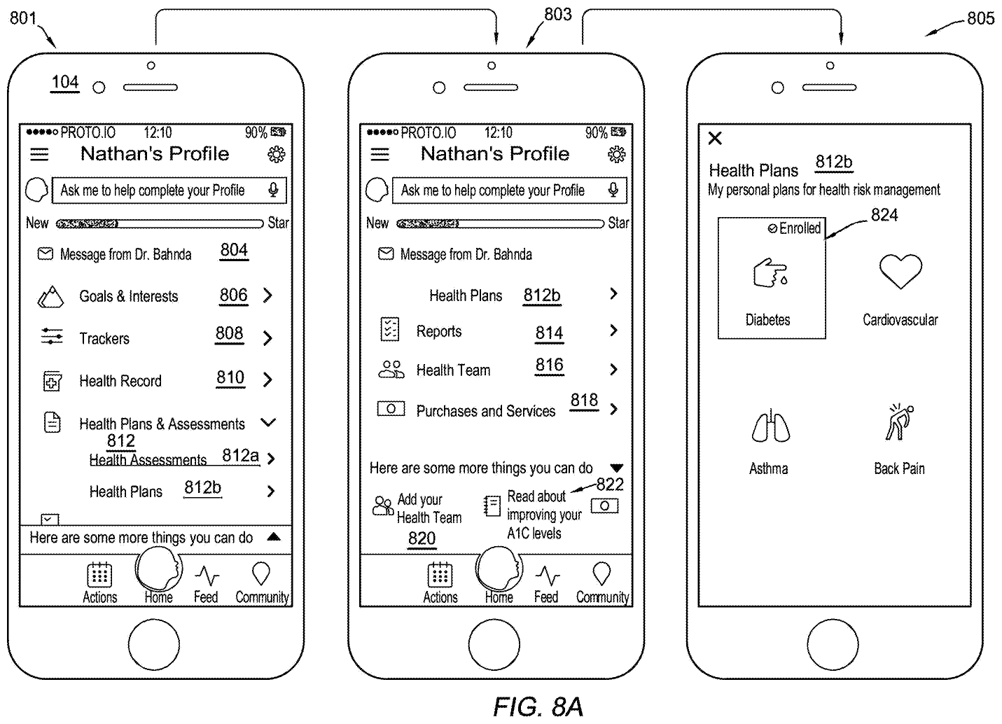

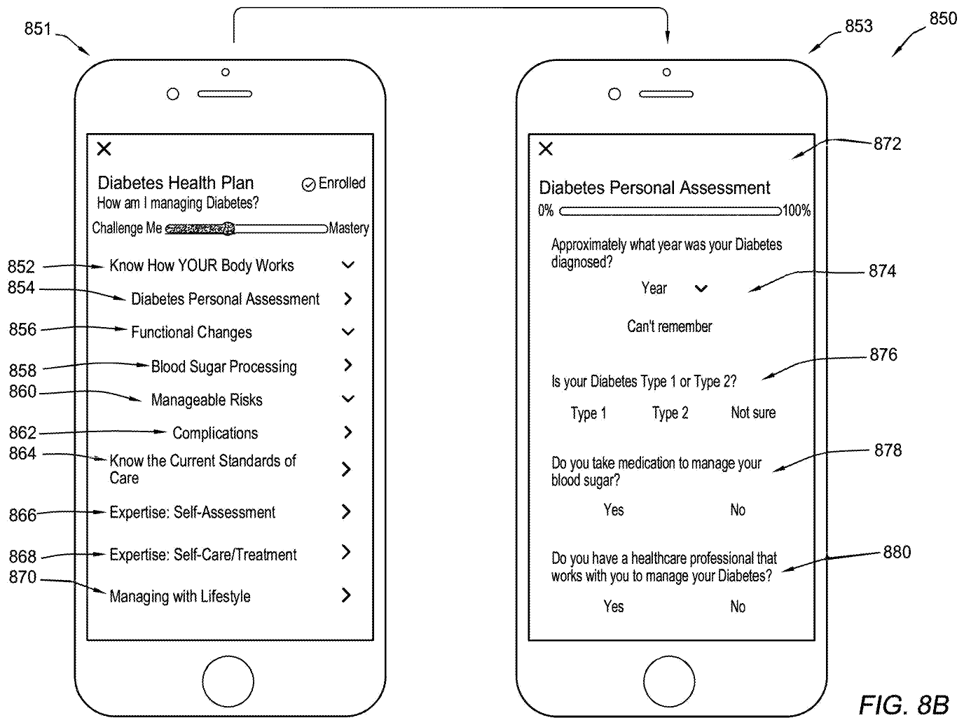

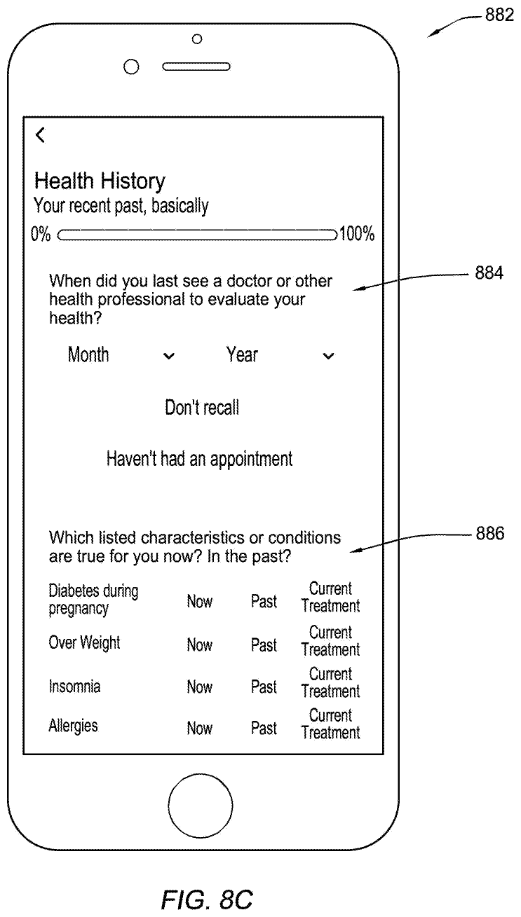

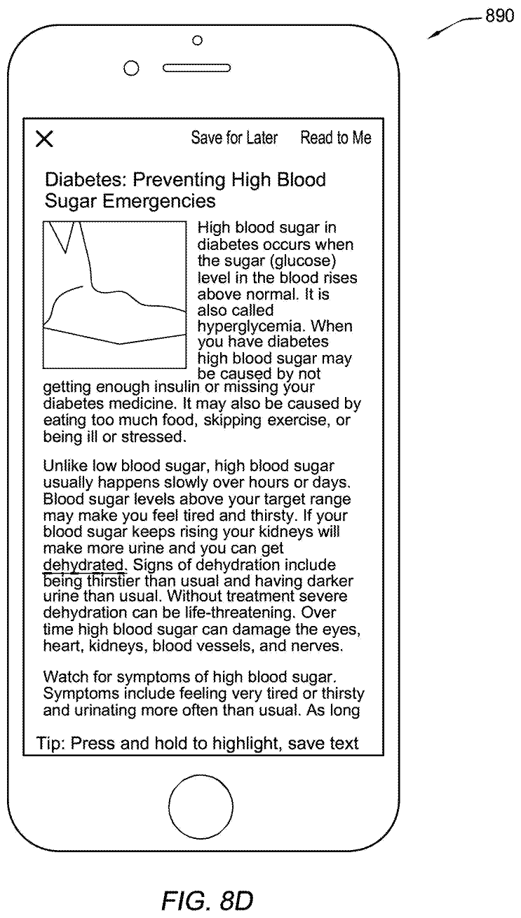

Next, the method includes prompting the user to build his profile (block 604). In various embodiments, building his profile includes displaying a GUI asking the user to enter in additional information, such as age, weight, height, and health concerns. In various embodiments, the steps of building a user profile is progressive, where building the user profile takes place over time. In some embodiments, the process of building the user profile is presented as a game. Where a user is presented with a ladder approach to create a “star profile”. Aspects of a graphical user interface presented during the profile building step are additionally discussed in FIGS. 8A-8B.

The method contemplates the build profile (block 604) method step is optional. For example, the user may complete building his profile at this method step 604, the user may complete his profile at a later time, or the cognitive intelligence platform 102 builds the user profile over time as more data about the user is received and processed. For example, the user is prompted to build his profile, however, the user fails to enter in information or skips the step. The method proceeds to prompting a user to complete a microsurvey (block 606). In some embodiments, the cognitive agent 110 uses answers received in response to the microsurvey to build the profile of the user. Overall, the data collected through the user registration process is stored and used later as available data to inform answers to missing parameters.

Next, the cognitive agent 110 proceeds to scheduling a service (block 608). The service can be scheduled such that it aligns with a health plan of the user or a protocol that results in a therapeutic goal. Next, the cognitive agent 110 proceeds to reaching agreement on a care plan (block 610).

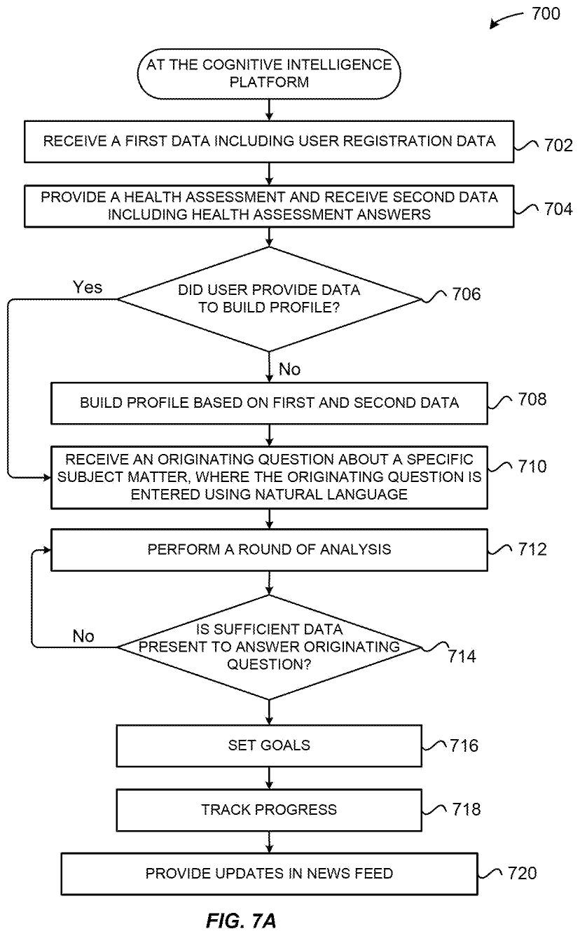

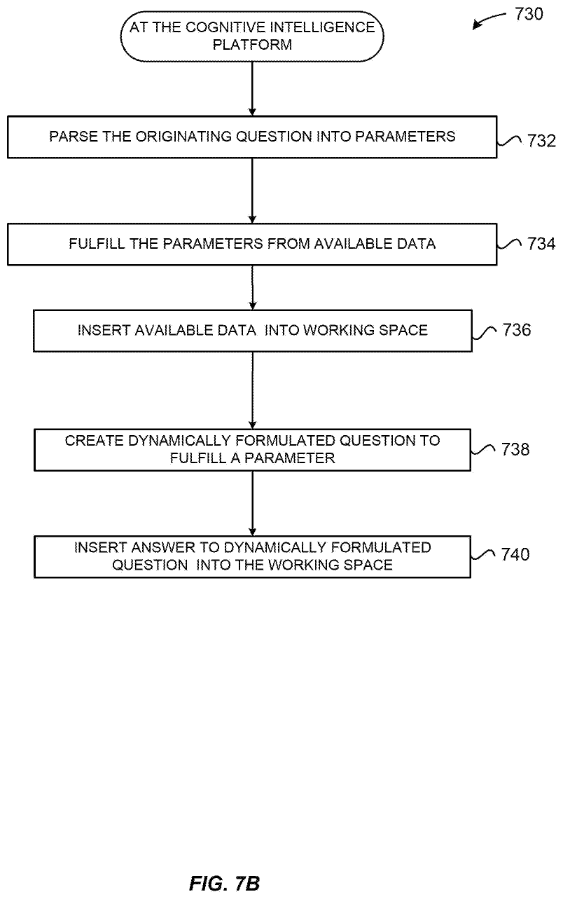

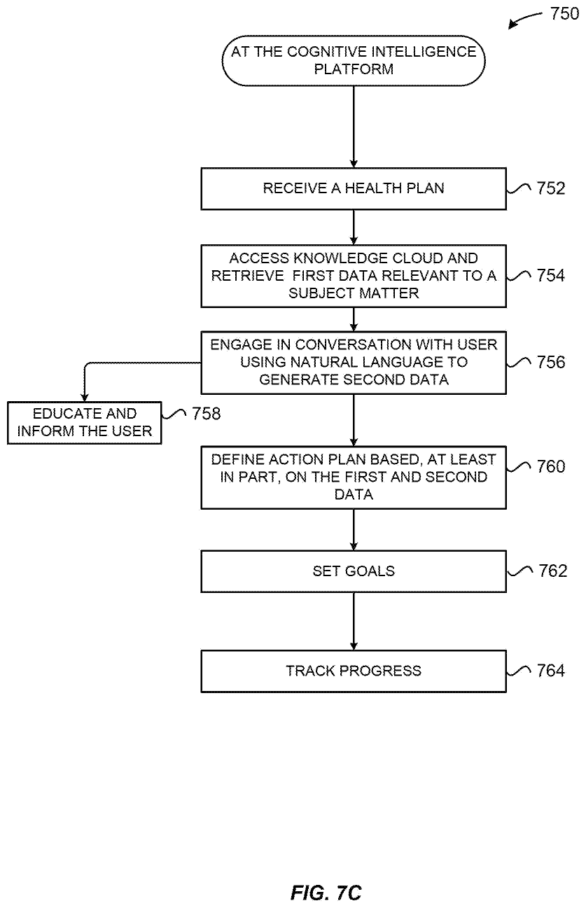

FIGS. 7A, 7B, and 7C, show methods, in accordance with various embodiments. The methods are performed at the cognitive intelligence platform. In particular, in FIG. 7A, the method begins with receiving a first data including user registration data (block 702); and providing a health assessment and receiving second data including health assessment answers (block 704). In various embodiments, the health assessment is a micro-survey with dynamically formulated questions presented to the user.

Next the method determine if the user provided data to build a profile (decision block 706). If the user did not provide data to build the profile, the method proceeds to building profile based on first and second data (block 708). If the user provided data to build the profile, the method proceeds to the next block (block 710).

At block 710, the method 700 proceeds to receiving an originating question about a specific subject matter, where the originating question is entered using natural language. In some embodiments, the originating question may be used to identify one or more care plans to recommend to the user, as will described further herein. Next, the method proceeds to performing a round of analysis (block 712). Next, the method determines if sufficient data is present to answer originating questions (decision block 714). If no, the method proceeds to block 712 and the method performs another round of analysis. If yes, the method proceeds to setting goals (block 716), then tracking progress (block 718), and then providing updates in a news feed (block 720).

In FIG. 7B, a method 730 of performing a round of analysis is illustrated. The method begins with parsing the originating question into parameters (block 732); fulfilling the parameters from available data (block 734); inserting available data (first data) into a working space (block 736); creating a dynamically formulated question to fulfill a parameter (block 738); and inserting an answer to the dynamically formulated question into the working space (block 740).

In FIG. 7C, a method 750 is performed at the cognitive intelligence platform. The method begins with receiving a health plan (block 752); accessing the knowledge cloud and retrieving first data relevant to the subject matter (block 754); and engaging in conversation with the user using natural language to general second data (block 756). In various embodiments, the second data can include information such as a user's scheduling preferences, lifestyle choices, and education level. During the process of engaging in conversation, the method includes educating and informing the user (block 758). Next, the method includes defining an action plan based, at least in part, on the first and second data (block 760); setting goals (block 762); and tracking progress (block 764). In some embodiments, the action plan may be defined based on content that is stored using the distributed ledger.

FIGS. 8A, 8B, 8C, and 8D illustrate aspects of interactions between a user and the cognitive intelligence platform 102, in accordance with various embodiments. As a user interacts with the GUI, the cognitive intelligence platform 102 continues to build a database of knowledge about the user based on questions asked by the user as well as answers provided by the user (e.g., available data as described in FIG. 4). In particular, FIG. 8A displays a particular screen shot 801 of the user device 104 at a particular instance in time. The screen shot 801 displays a graphical user interface (GUI) with menu items associated with a user's (e.g., Nathan) profile including Messages from the doctor (element 804), Goals (element 806), Trackers (element 808), Health Record (element 810), and Health Plans & Assessments (element 812). The menu item Health Plans & Assessments (element 812), additionally include child menu items: Health Assessments (element 812a), Health plans (812b).

The screen shot 803 displays the same GUI as in the screen shot 801, however, the user has scrolled down the menu, such that additional menu items below Health Plans & Assessments (element 812) are shown. The additional menu items include Reports (element 814), Health Team (element 816), and Purchases and Services (Element 818). Furthermore, additional menu items include Add your Health Team (element 820) and Read about improving your A1C levels (element 822).

For purposes of the example in FIG. 8A, the user selects the menu item Health Plans (element 812b). Accordingly, in response to the receiving the selection of the menu item Health Plans, types of health plans are shown, as illustrated in screen shot 805. The types of health plans shown with respect to Nathan's profile include: Diabetes (element 824), Cardiovascular, Asthma, and Back Pain. Each type of health plan leads to separate displays. For purposes of this example in FIG. 8A, the user selects the Diabetes (element 824) health plan.

In FIG. 8B, the screenshot 851 is seen in response to the user's selection of Diabetes (element 824). Example elements displayed in screenshot 851 include: Know How YOUR Body Works (element 852); Know the Current Standards of Care (element 864); Expertise: Self-Assessment (element 866); Expertise: Self-Care/Treatment (element 868); and Managing with Lifestyle (element 870). Managing with Lifestyle (element 870) focuses and tracks actions and lifestyle actions that a user can engage in. As a user's daily routine helps to manage diabetes, managing the user's lifestyle is important. The cognitive agent 110 can align a user's respective health plan based on a health assessment at enrollment. In various embodiments, the cognitive agent 110 aligns the respective health plan with an interest of the user, a goal and priority of the user, and lifestyle factors of the user-including exercise, diet and nutrition, and stress reduction.

Each of these elements 852, 864, 866, 868, and 870 can display additional sub-elements depending on a selection of the user. For example, as shown in the screen shot 851, Know How YOUR Body Works (element 852) includes additional sub-elements: Diabetes Personal Assessment (854); and Functional Changes (856). Additional sub-elements under Functional Changes (856) include: Blood Sugar Processing (858) and Manageable Risks (860). Finally, the sub-element Manageable Risks (860) includes an additional sub-element Complications (862). For purposes of this example, the user selects the Diabetes Personal Assessment (854) and the screen shot 853 shows a GUI (872) associated with the Diabetes Personal Assessment.

The Diabetes Personal Assessment includes questions such as “Approximately what year was your Diabetes diagnosed” and corresponding elements a user can select to answer including “Year” and “Can't remember” (element 874). Additional questions include “Is your Diabetes Type 1 or Type 2” and corresponding answers selectable by a user include “Type 1,” “Type 2,” and “Not sure” (element 876). Another question includes “Do you take medication to manage your blood sugar” and corresponding answers selectable by a user include “Yes” and “No” (element 878). An additional question asks “Do you have a healthcare professional that works with you to manage your Diabetes” and corresponding answers selectable by the user include “Yes” and “No” (element 880).

In various embodiments, the cognitive intelligence platform 102 collects information about the user based on responses provided by the user or questions asked by the user as the user interacts with the GUI. For example, as the user views the screen shot 851, if the user asks if diabetes is curable, this question provides information about the user such as a level of education of the user.

FIG. 8C illustrates aspects of an additional tool—e.g., a microsurvey-provided to the user that helps gather additional information about the user (e.g., available data). In various embodiments, a micro-survey represent a short targeted survey, where the questions presented in the survey are limited to a respective micro-theory. A microsurvey can be created by the cognitive intelligence platform 102 for several different purposes, including: completing a user profile, and informing a missing parameter during the process of answering an originating question.

In FIG. 8C, the microsurvey 882 gathers information related to health history, such as “when did you last see a doctor or other health professional to evaluate your health” where corresponding answers selectable by the user include specifying a month and year, “don't recall,” and “haven't had an appointment” (element 884). An additional question asks “Which listed characteristics or conditions are true for you now? In the past?” where corresponding answers selectable by the user include “Diabetes during pregnancy,” “Over Weight,” “Insomnia,” and “Allergies” (element 886). Each of the corresponding answer in element 886 also includes the option to indicate whether the characteristics or conditions are true for the user “Now”, “Past,” or “Current Treatment.”

In FIG. 8D, aspects of educating a user are shown in the screen shot 890. The screen shot displays an article titled “Diabetes: Preventing High Blood Sugar Emergencies,” and proceeds to describe when high blood sugar occurs and other information related to high blood sugar. The content displayed in the screen shot 890 is searchable and hearable as a podcast.

Accordingly, the cognitive agent 110 can answer a library of questions and provide content for many questions a user has as it related to diabetes. The information provided for purposes of educating a user is based on an overall health plan of the user, which is based on metadata analysis of interactions with the user, and an analysis of the education level of the user. In some embodiments, the cognitive agent 110 may be used to answer questions relating to content stored using the distributed ledger.

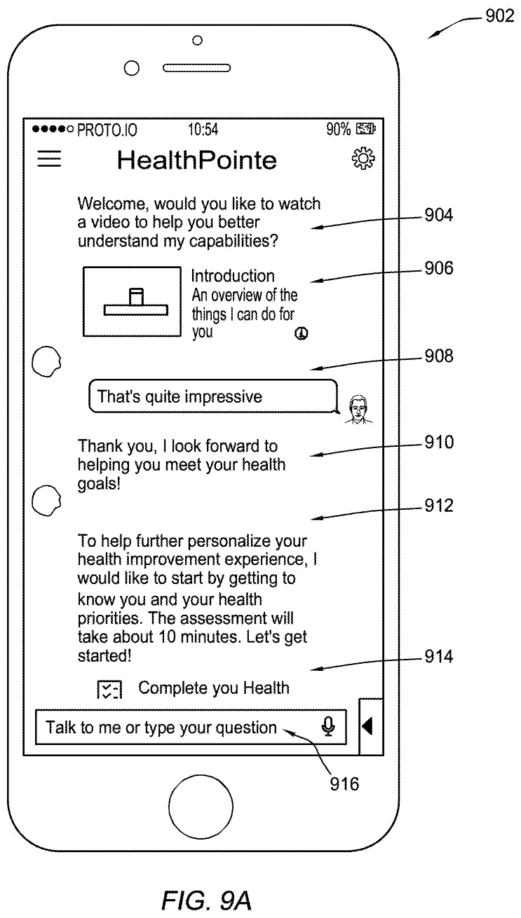

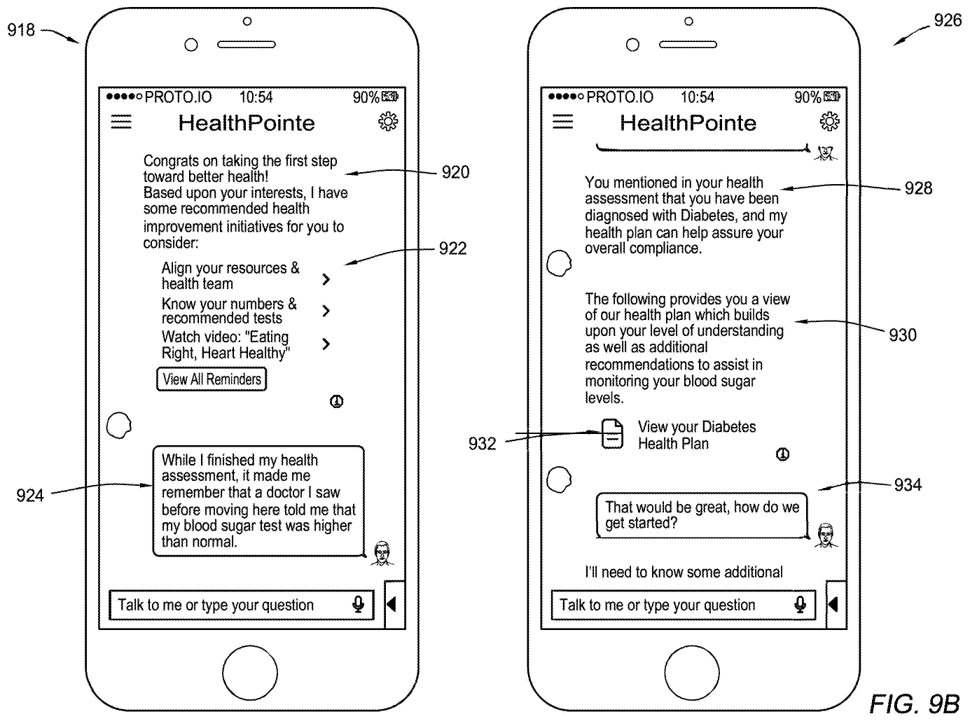

FIGS. 9A-9B illustrate aspects of a conversational stream, in accordance with various embodiments. In particular, FIG. 9A displays an example conversational stream between a user and the cognitive agent 110. The screen shot 902 is an example of a dialogue that unfolds between a user and the cognitive agent 110, after the user has registered with the cognitive intelligence platform 102. In the screen shot 902, the cognitive agent 110 begins by stating “Welcome, would you like to watch a video to help you better understand my capabilities” (element 904). The cognitive agent provides an option to watch the video (element 906). In response, the user inputs text “that's quite impressive” (element 908). In various embodiments, the user inputs text using the input box 916, which instructs the user to “Talk to me or type your question”.

Next, the cognitive agent 110 says “Thank you. I look forward to helping you meet your health goals!” (element 910). At this point, the cognitive agent 110 can probe the user for additional data by offering a health assessment survey (e.g., a microsurvey) (element 914). The cognitive agent 110 prompts the user to fill out the health assessment by stating: “To help further personalize your health improvement experience, I would like to start by getting to know you and your health priorities. The assessment will take about 10 minutes. Let's get started!” (element 912).

In FIG. 9B, an additional conversational stream between the user and the cognitive agent 110 is shown. In this example conversational stream, the user previously completed a health assessment survey. The conversational stream can follow the example conversational stream discussed in FIG. 9A.

In the screen shot 918, the cognitive agent acknowledges the user's completion of the health assessment survey (element 920) and provides additional resources to the user (element 922). In element 920, the cognitive agent states: “Congrats on taking the first step toward better health! Based upon your interest, I have some recommended health improvement initiatives for you to consider,” and presents the health improvement initiatives. In the example conversational stream, the user gets curious about a particular aspect of his health and states: “While I finished my health assessment, it made me remember that a doctor I saw before moving here told me that my blood sugar test was higher than normal.” (element 924). After receiving the statement in element 924, the cognitive agent 110 treats the statement as an originating question and undergoes an initial round of analysis (and additional rounds of analysis as needed) as described above.

The cognitive agent 110 presents an answer as shown in screen shot 926. For example, the cognitive agent 110 states: “You mentioned in your health assessment that you have been diagnosed with Diabetes, and my health plan can help assure your overall compliance” (element 928). The cognitive agent further adds: “The following provides you a view of our health plan which builds upon your level of understanding as well as additional recommendations to assist in monitoring your blood sugar levels” (element 930). The cognitive agent 110 provides the user with the option to view his Diabetes Health Plan (element 932).

The user responds “That would be great, how do we get started” (element 934). The cognitive agent 110 receives the user's response as another originated question and undergoes an initial round of analysis (and additional rounds of analysis as needed) as described above. In the example screen shot 926, the cognitive agent 110 determines additional information is needed and prompts the user for additional information. In some embodiments, the cognitive agent 110 may be used to respond to questions that the user may ask to identify optimal content to view (e.g., an optimal care plan).

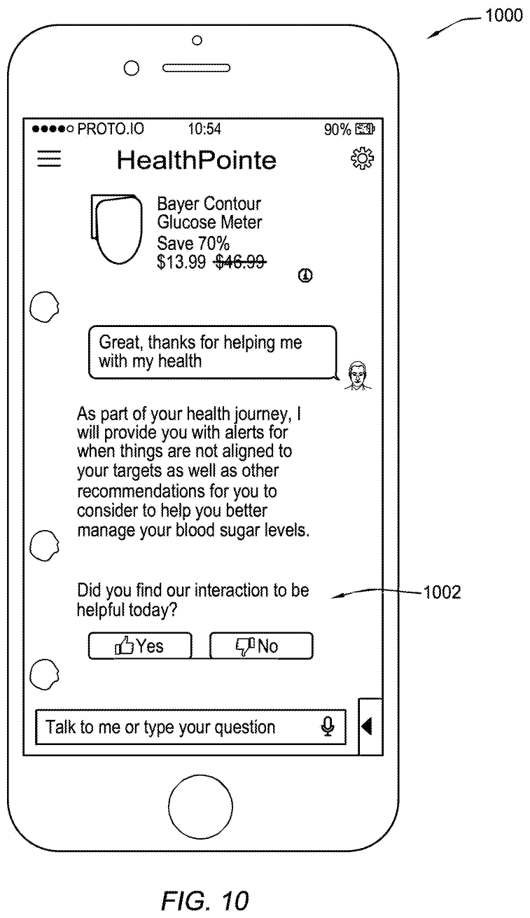

FIG. 10 illustrates an additional conversational stream, in accordance with various embodiments. In particular, in the screen shot 1000, the cognitive agent 110 elicit feedback (element 1002) to determine whether the information provided to the user was useful to the user.

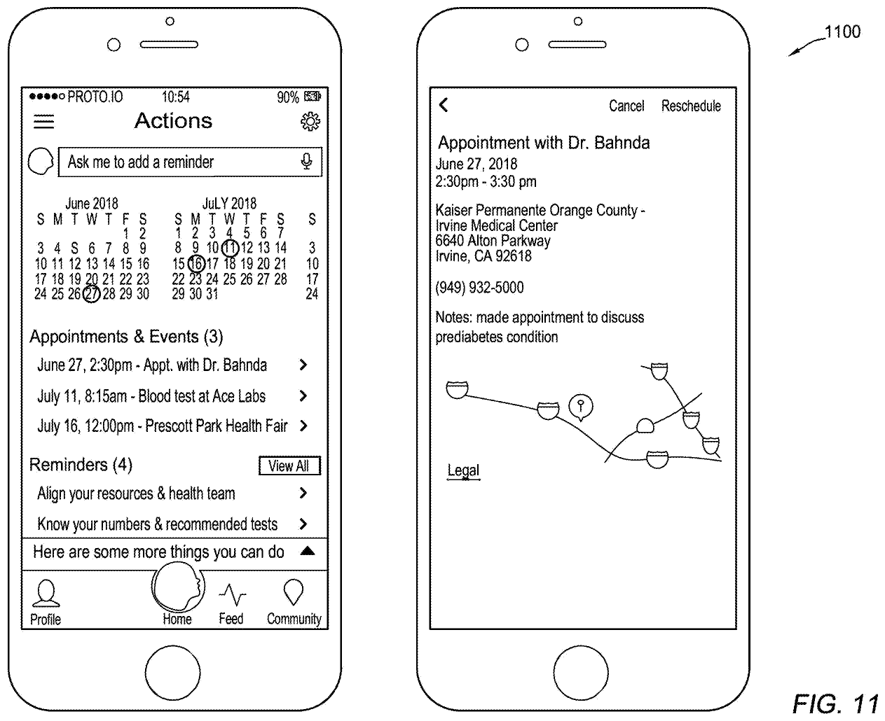

FIG. 11 illustrates aspects of an action calendar, in accordance with various embodiments. The action calendar is managed through the conversational stream between the cognitive agent 110 and the user. The action calendar aligns to care and wellness protocols, which are personalized to the risk condition or wellness needs of the user. The action calendar is also contextually aligned (e.g., what is being required or searched by the user) and hyper local (e.g., aligned to events and services provided in the local community specific to the user).

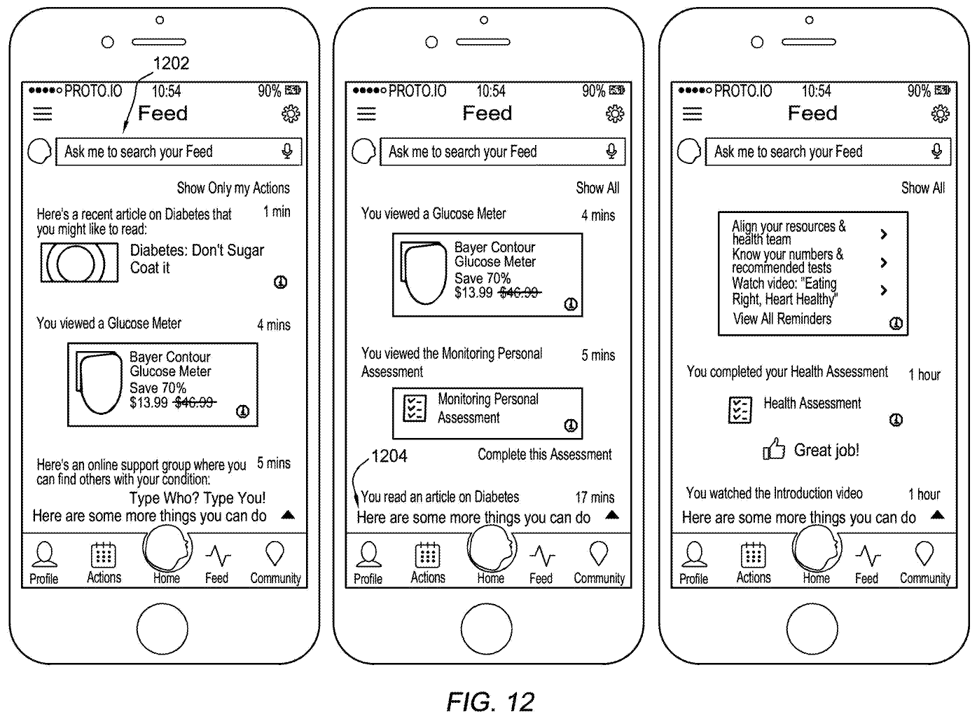

FIG. 12 illustrates aspects of a feed, in accordance with various embodiments. The feed allows a user to explore new opportunities and celebrate achieving goals (e.g., therapeutic or wellness goals). The feed provides a searchable interface (element 1202).

The feed provides an interface where the user accesses a personal log of activities the user is involved in. The personal log is searchable. For example, if the user reads an article recommended by the cognitive agent 110 and highlights passages, the highlighted passages are accessible through the search. Additionally, the cognitive agent 110 can initiate a conversational stream focused on subject matter related to the highlighted passages.

The feed provides an interface to celebrate mini achievements and successes in the user's personal goals (e.g., therapeutic or wellness goals). In the feed, the cognitive agent 110 is still available (ribbon 1204) to help search, guide, or steer the user toward a therapeutic or wellness goal.

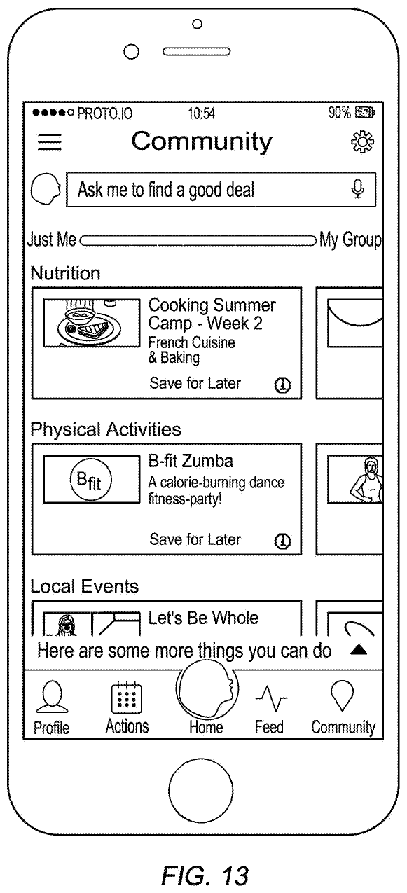

FIG. 13 illustrates aspects of a hyper-local community, in accordance with various embodiments. A hyper-local community is a digital community that is health and wellness focused and encourages the user to find opportunities for themselves and get involved in a community that is physically close to the user. The hyper-local community allows a user to access a variety of care and wellness resources within his community and example recommendations include: Nutrition; Physical Activities; Healthcare Providers; Educations; Local Events; Services; Deals and Stores; Charities; and Products offered within the community. The cognitive agent 110 optimizes suggestions which help the user progress towards a goal as opposed to providing open ended access to hyper-local assets. The recommendations are curated and monitored for relevance to the user, based on the user's goals and interactions between the user and the cognitive agent 110.

Accordingly, the cognitive intelligence platform provides several core features including:
1) the ability to identify an appropriate action plan using narrative style interactions that generates data that includes intent and causation and using narrative style interactions;
2) monitoring: integration of offline to online clinical results across the functional medicine clinical standards;
3) the knowledge cloud that includes a comprehensive knowledge base of thousands of health related topics, an educational guide to better health aligned to western and eastern culture;
4) coaching using artificial intelligence; and
5) profile and health store that offers a holistic profile of each consumers health risks and interactions, combined with a repository of services, products, lab tests, devices, deals, supplements, pharmacy & telemedicine.

In some embodiments, the identified action plan, a monitoring record based on the monitoring, content stored using the knowledge cloud, a coaching record based on the coaching, and/or profile and health store data may be stored using the distributed ledger (and subsequently accessed by permitted users).

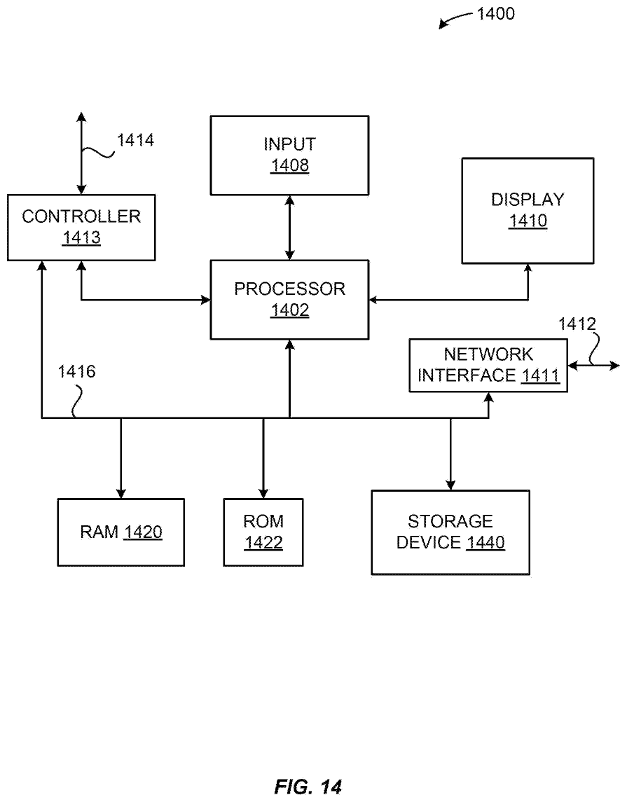

FIG. 14 illustrates a detailed view of a computing device 1400 that can be used to implement the various components described herein, according to some embodiments. In particular, the detailed view illustrates various components that can be included in the user device 104 illustrated in FIG. 1, as well as the several computing devices implementing the cognitive intelligence platform 102. The computing device 1400 may also be used by the service provider 112 and/or the facility 114. As shown in FIG. 14, the computing device 1400 can include a processor 1402 that represents a microprocessor or controller for controlling the overall operation of the computing device 1400. The computing device 1400 can also include a user input device 1408 that allows a user of the computing device 1400 to interact with the computing device 1400. For example, the user input device 1408 can take a variety of forms, such as a button, keypad, dial, touch screen, audio input interface, visual/image capture input interface, input in the form of sensor data, and so on. Still further, the computing device 1400 can include a display 1410 that can be controlled by the processor 1402 to display information to the user. A data bus 1416 can facilitate data transfer between at least a storage device 1440, the processor 1402, and a controller 1413. The controller 1413 can be used to interface with and control different equipment through an equipment control bus 1414. The computing device 1400 can also include a network/bus interface 1411 that couples to a data link 1412. In the case of a wireless connection, the network/bus interface 1411 can include a wireless transceiver.

As noted above, the computing device 1400 also includes the storage device 1440, which can comprise a single disk or a collection of disks (e.g., hard drives), and includes a storage management module that manages one or more partitions within the storage device 1440. In some embodiments, storage device 1440 can include flash memory, semiconductor (solid-state) memory or the like. The computing device 1400 can also include a Random-Access Memory (RAM) 1420 and a Read-Only Memory (ROM) 1422. The ROM 1422 can store programs, utilities or processes to be executed in a non-volatile manner. The RAM 1420 can provide volatile data storage, and stores instructions related to the operation of processes and applications executing on the computing device.

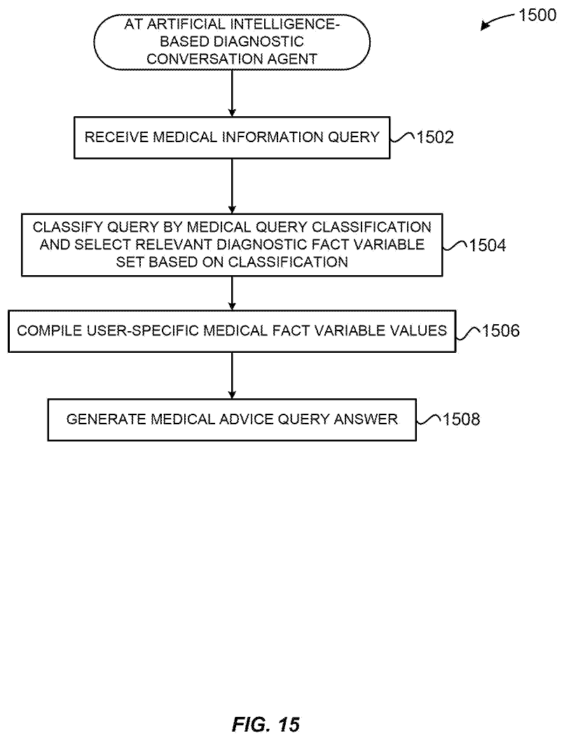

FIG. 15 shows a method (1500), in accordance with various embodiments, for answering a user-generated natural language medical information query based on a diagnostic conversational template.

In the method as shown in FIG. 15, an artificial intelligence-based diagnostic conversation agent receives a user-generated natural language medical information query as entered by a user through a user interface on a computer device (FIG. 15, block 1502). In some embodiments, the artificial intelligence-based diagnostic conversation agent is the conversation agent 110 of FIG. 1. In some embodiments the computer device is the mobile device 104 of FIG. 1. One example of a user-generated natural language medical information query as entered by a user through a user interface is the question “Is a blood sugar of 90 normal?” as shown in line 402 of FIG. 4. In some embodiments, receiving a user-generated natural language medical information query as entered by a user through a user interface on a computer device (FIG. 15, block 1502) is Step 1 as earlier discussed in the context of “Analyzing Conversational Context As Part of Conversational Analysis”.

In response to the user-generated natural language medical information query, the artificial intelligence-based diagnostic conversation agent selects a diagnostic fact variable set relevant to generating a medical advice query answer for the user-generated natural language medical information query by classifying the user-generated natural language medical information query into one of a set of domain-directed medical query classifications associated with respective diagnostic fact variable sets (FIG. 15, block 1504). In some embodiments, the artificial intelligence-based diagnostic conversation agent selecting a diagnostic fact variable set relevant to generating a medical advice query answer for the user-generated natural language medical information query by classifying the user-generated natural language medical information query into one of a set of domain-directed medical query classifications associated with respective diagnostic fact variable sets (FIG. 15, block 1504) is accomplished through one or more of Steps 2-6 as earlier discussed in the context of “Analyzing Conversational Context As Part of Conversational Analysis”.

FIG. 15 further shows compiling user-specific medical fact variable values for one or more respective medical fact variables of the diagnostic fact variable set (FIG. 15, block 1506). Compiling user-specific medical fact variable values for one or more respective medical fact variables of the diagnostic fact variable set (FIG. 15, block 1506) may include one or more of Steps 2-6 as earlier discussed in the context of “Analyzing Conversational Context As Part of Conversational Analysis”.

In response to the user-specific medical fact variable values, the artificial intelligence-based diagnostic conversation agent generates a medical advice query answer in response to the user-generated natural language medical information query (FIG. 15, block 1508). In some embodiments, this is Step 7 as earlier discussed in the context of “Analyzing Conversational Context As Part of Conversational Analysis”.

In some embodiments, compiling user-specific medical fact variable values (FIG. 15, block 1506) includes extracting a first set of user-specific medical fact variable values from a local user medical information profile associated with the user-generated natural language medical information query and requesting a second set of user specific medical fact variable values through natural-language questions sent to the user interface on the mobile device (e.g. the microsurvey data 206 of FIG. 2 that came from the microsurvey 116 of FIG. 1). The local user medical information profile can be the profile as generated in FIG. 7A at block 708.

In some embodiments, compiling user-specific medical fact variable values (FIG. 15, block 1506) includes extracting a third set of user-specific medical fact variable values that are lab result values from the local user medical information profile associated with the user generated natural language medical information query. The local user medical information profile can be the profile as generated in FIG. 7A at block 708.

In some embodiments, compiling user-specific medical fact variable values (FIG. 15, block 1506) includes extracting a fourth set of user-specific medical variable values from a remote medical data service profile associated with the local user medical information profile. The remote medical data service profile can be the service provider data 202 of FIG. 2, which can come from the service provider 112 of FIG. 1. The local user medical information profile can be the profile as generated in FIG. 7A at block 708.

In some embodiments, compiling user-specific medical fact variable values (FIG. 15, block 1506) includes extracting a fifth set of user-specific medical variable values from demographic characterizations provided by a remote data service analysis of the local user medical information profile. The remote demographic characterizations can be the service provider data 202 of FIG. 2, which can come from the service provider 112 of FIG. 1. The local user medical information profile can be the profile as generated in FIG. 7A at block 708.

In some embodiments, generating the medical advice query answer (FIG. 15, block 1508) includes providing a treatment action-item recommendation in response to user-specific medical fact values that may be non-responsive to the medical question presented in the user-generated natural language medical information query. Such an action could define an action plan based on the data compiled (FIG. 15, block 1506), as shown in FIG. 7C, block 758.

In some embodiments, generating the medical advice query answer (FIG. 15, block 1506) includes providing a medical education media resource in response to user-specific medical fact variable values that may be non-responsive to the medical question presented in the user-generated natural language medical information query. Such an action could serve to educate and inform the user, as in block 758 of FIG. 7C.

In some embodiments, selecting a diagnostic fact variable set relevant to generating a medical advice query answer for the user-generated natural language medical information query by classifying the user-generated natural language medical information query into one of a set of domain-directed medical query classifications associated with respective diagnostic fact variable sets (FIG. 15, block 1504) includes classifying the user-generated natural language medical information query into one of a set of domain-directed medical query classifications based on relevance to the local user medical information profile associated with the user-generated natural language medical information query. The local user medical information profile can be the profile as generated in FIG. 7A at block 708.

In some embodiments, the method (1500) for answering a user-generated natural language medical information query based on a diagnostic conversational template is implemented as a computer program product in a computer-readable medium.

In some embodiments, the system and method 1500 shown in FIG. 15 and described above is implemented on the computing device 1400 shown in FIG. 14.

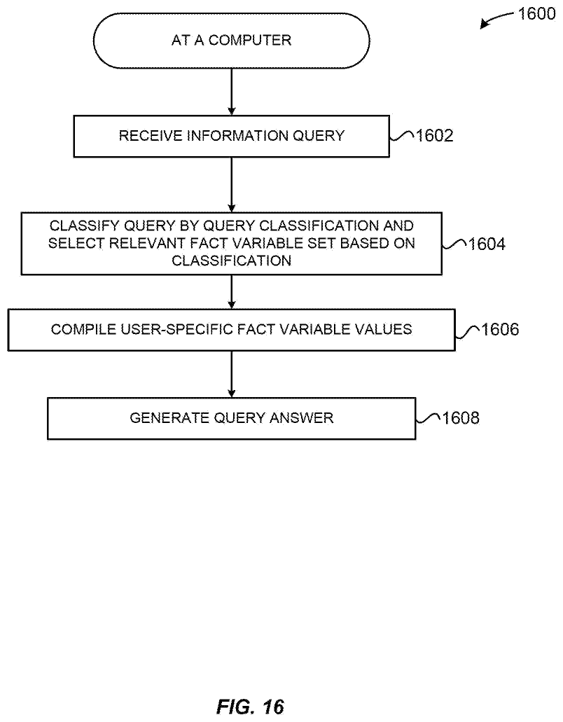

FIG. 16 shows a method (1600), in accordance with various embodiments, for answering a user-generated natural language query based on a conversational template.

In the method as shown in FIG. 16, an artificial intelligence-based conversation agent receives a user-generated natural language query as entered by a user through a user interface (FIG. 16, block 1602). In some embodiments, the artificial intelligence-based conversation agent is the conversation agent 110 of FIG. 1. In some embodiments, the user interface is on a computer device. In some embodiments the computer device is the mobile device 104 of FIG. 1. One example of a user-generated natural language query as entered by a user through a user interface is the question “Is a blood sugar of 90 normal?” as shown in line 402 of FIG. 4. In some embodiments, receiving a user-generated natural language query as entered by a user through a user interface on a computer device (FIG. 16, block 1602) is Step 1 as earlier discussed in the context of “Analyzing Conversational Context As Part of Conversational Analysis”.

In response to the user-generated natural language query, the artificial intelligence-based conversation agent selects a fact variable set relevant to generating a query answer for the user-generated natural language query by classifying the user-generated natural language query into one of a set of domain-directed query classifications associated with respective fact variable sets (FIG. 16, block 1604). In some embodiments, the artificial intelligence-based conversation agent selecting a fact variable set relevant to generating a query answer for the user-generated natural language query by classifying the user-generated natural language query into one of a set of domain-directed query classifications associated with respective fact variable sets (FIG. 16, block 1604) is accomplished through one or more of Steps 2-6 as earlier discussed in the context of “Analyzing Conversational Context As Part of Conversational Analysis”.

FIG. 16 further shows compiling user-specific variable values for one or more respective fact variables of the fact variable set (FIG. 16, block 1606). Compiling user-specific fact variable values for one or more respective fact variables of the fact variable set (FIG. 16, block 1606) may include one or more of Steps 2-6 as earlier discussed in the context of “Analyzing Conversational Context As Part of Conversational Analysis”.

In response to the user-specific fact variable values, the artificial intelligence-based conversation agent generates a query answer in response to the user-generated natural language query (FIG. 16, block 1608). In some embodiments, this is Step 7 as earlier discussed in the context of “Analyzing Conversational Context As Part of Conversational Analysis”.

In some embodiments, compiling user-specific fact variable values (FIG. 16, block 1606) includes extracting a first set of user-specific fact variable values from a local user profile associated with the user-generated natural language query and requesting a second set of user specific variable values through natural-language questions sent to the user interface on the mobile device (e.g. the microsurvey data 206 of FIG. 2 that came from the microsurvey 116 of FIG. 1). The local user profile can be the profile as generated in FIG. 7A at block 708. In some embodiments, the natural language questions sent to the user interface on the mobile device can be a part of a conversation template.

In some embodiments, compiling user-specific fact variable values (FIG. 16, block 1606) includes extracting a third set of user-specific fact variable values that are test result values from the local user profile associated with the user generated natural language query. The local user profile can be the profile as generated in FIG. 7A at block 708. In some embodiments, compiling user-specific fact variable values (FIG. 16, block 1606) includes extracting a fourth set of user-specific variable values from a remote data service profile associated with the local user profile. The remote data service profile can be the service provider data 202 of FIG. 2, which can come from the service provider 112 of FIG. 1. The local user profile can be the profile as generated in FIG. 7A at block 708.

In some embodiments, compiling user-specific fact variable values (FIG. 16, block 1606) includes extracting a fifth set of user-specific variable values from demographic characterizations provided by a remote data service analysis of the local user profile. The remote demographic characterizations can be the service provider data 202 of FIG. 2, which can come from the service provider 112 of FIG. 1. The local user profile can be the profile as generated in FIG. 7A at block 708.

In some embodiments, generating the query answer (FIG. 16, block 1608) includes providing an action-item recommendation in response to user-specific fact values that may be non-responsive to the question presented in the user-generated natural language query. Such an action could define an action plan based on the data compiled (FIG. 16, block 1606), as shown in FIG. 7C, block 758.

In some embodiments, generating the advice query answer (FIG. 16, block 1606) includes providing an education media resource in response to user-specific fact variable values that may be non-responsive to the question presented in the user-generated natural language query. Such an action could serve to educate and inform the user, as in block 758 of FIG. 7C.

In some embodiments, selecting a fact variable set relevant to generating a query answer for the user-generated natural language query by classifying the user-generated natural language query into one of a set of domain-directed query classifications associated with respective fact variable sets (FIG. 16, block 1604) includes classifying the user-generated natural language query into one of a set of domain-directed query classifications based on relevance to the local user profile associated with the user-generated natural language query. The local user profile can be the profile as generated in FIG. 7A at block 708.

In some embodiments, the method (1600) for answering a user-generated natural language query based on a conversational template is implemented as a computer program product in a computer-readable medium.

In some embodiments, the system and method shown in FIG. 16 and described above is implemented in the cognitive intelligence platform 102 shown in FIG. 1.

In the cognitive intelligence platform 102, a cognitive agent 110 is configured for receiving a user-generated natural language query at an artificial intelligence-based conversation agent from a user interface on a user device 104 (FIG. 16, block 1602).

A critical thinking engine 108 is configured for, responsive to content of the user-generated natural language query, selecting a fact variable set relevant to generating a query answer for the user-generated natural language query by classifying the user-generated natural language query into one of a set of domain-directed query classifications associated with respective fact variable sets (FIG. 16, block 1604).

Included is a knowledge cloud 106 that compiles user-specific fact variable values for one or more respective fact variables of the fact variable set (FIG. 16, block 1606).

Responsive to the fact variable values, the cognitive agent 110 is further configured for generating the query answer in response to the user-generated natural language query (FIG. 16, block 1606).

In some embodiments, the system and method 1600 shown in FIG. 16 and described above is implemented on the computing device 1400 shown in FIG. 14.

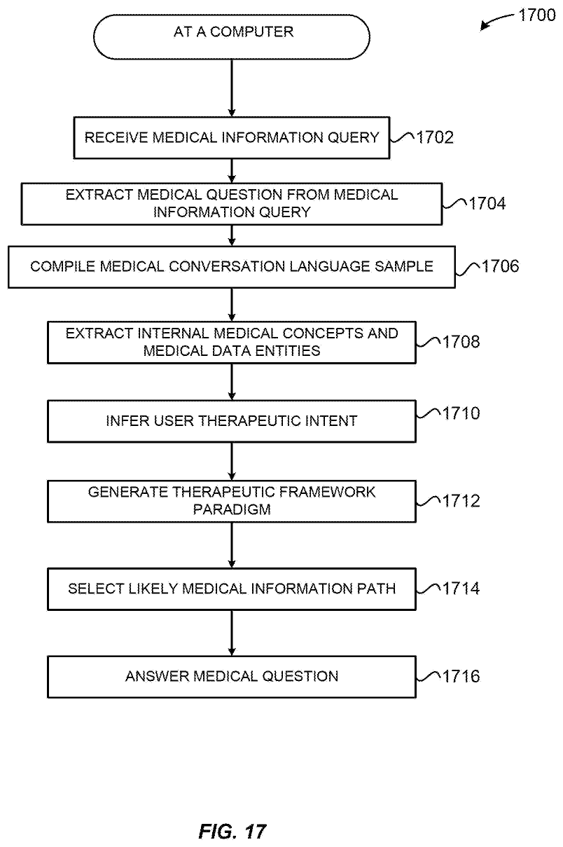

FIG. 17 shows a computer-implemented method 1700 for answering natural language medical information questions posed by a user of a medical conversational interface of a cognitive artificial intelligence system. In some embodiments, the method 1700 is implemented on a cognitive intelligence platform. In some embodiments, the cognitive intelligence platform is the cognitive intelligence platform 102 as shown in FIG. 1. In some embodiments, the cognitive intelligence platform is implemented on the computing device 1400 shown in FIG. 14.

The method 1700 involves receiving a user-generated natural language medical information query from a medical conversational user interface at an artificial intelligence-based medical conversation cognitive agent (block 1702). In some embodiments, receiving a user-generated natural language medical information query from a medical conversational user interface at an artificial intelligence-based medical conversation cognitive agent (block 1702) is performed by a cognitive agent that is a part of the cognitive intelligence platform and is configured for this purpose. In some embodiments, the artificial intelligence-based diagnostic conversation agent is the conversation agent 110 of FIG. 1. One example of a user-generated natural language medical information query is “Is a blood sugar of 90 normal?” as shown in line 402 of FIG. 4. In some embodiments, the user interface is on the mobile device 104 of FIG. 1. In some embodiments, receiving a user-generated natural language medical information query from a medical conversational user interface at an artificial intelligence-based medical conversation cognitive agent (block 1702) is Step 1 as earlier discussed in the context of “Analyzing Conversational Context As Part of Conversational Analysis”.

The method 1700 further includes extracting a medical question from a user of the medical conversational user interface from the user-generated natural language medical information query (block 1704). In some embodiments, extracting a medical question from a user of the medical conversational user interface from the user-generated natural language medical information query (block 1704) is performed by a critical thinking engine configured for this purpose. In some embodiments, the critical thinking engine is the critical thinking engine 108 of FIG. 1. In some embodiments, extracting a medical question from a user of the medical conversational user interface from the user-generated natural language medical information query (block 1704) is accomplished through one or more of Steps 2-6 as earlier discussed in the context of “Analyzing Conversational Context As Part of Conversational Analysis”.

The method 1700 includes compiling a medical conversation language sample (block 1706). In some embodiments, compiling a medical conversation language sample (block 1706) is performed by a critical thinking engine configured for this purpose. In some embodiments, the critical thinking engine is the critical thinking engine 108 of FIG. 1. The medical conversation language sample can include items of health-information-related-text derived from a health-related conversation between the artificial intelligence-based medical conversation cognitive agent and the user. In some embodiments compiling a medical conversation language sample (block 1706) is accomplished through one or more of Steps 2-6 as earlier discussed in the context of “Analyzing Conversational Context As Part of Conversational Analysis”.

The method 1700 involves extracting internal medical concepts and medical data entities from the medical conversation language sample (block 1708). In some embodiments, extracting internal medical concepts and medical data entities from the medical conversation language sample (block 1708) is performed by a critical thinking engine configured for this purpose. In some embodiments, the critical thinking engine is the critical thinking engine 108 of FIG. 1. The internal medical concepts can include descriptions of medical attributes of the medical data entities. In some embodiments, extracting internal medical concepts and medical data entities from the medical conversation language sample (block 1708) is accomplished through one or more of Steps 2-6 as earlier discussed in the context of “Analyzing Conversational Context As Part of Conversational Analysis”.

The method 1700 involves inferring a therapeutic intent of the user from the internal medical concepts and the medical data entities (block 1710). In some embodiments, inferring a therapeutic intent of the user from the internal medical concepts and the medical data entities (block 1710) is performed by a critical thinking engine configured for this purpose. In some embodiments, the critical thinking engine is the critical thinking engine 108 of FIG. 1. In some embodiments, inferring a therapeutic intent of the user from the internal medical concepts and the medical data entities (block 1710) is accomplished as in Step 2 as earlier discussed in the context of “Analyzing Conversational Context As Part of Conversational Analysis”.

The method 1700 includes generating a therapeutic paradigm logical framework 1800 for interpreting of the medical question (block 1712). In some embodiments, generating a therapeutic paradigm logical framework 1800 for interpreting of the medical question (block 1712) is performed by a critical thinking engine configured for this purpose. In some embodiments, the critical thinking engine is the critical thinking engine 108 of FIG. 1. In some embodiments, generating a therapeutic paradigm logical framework 1800 for interpreting of the medical question (block 1712) is accomplished as in Step 5 as earlier discussed in the context of “Analyzing Conversational Context As Part of Conversational Analysis”.

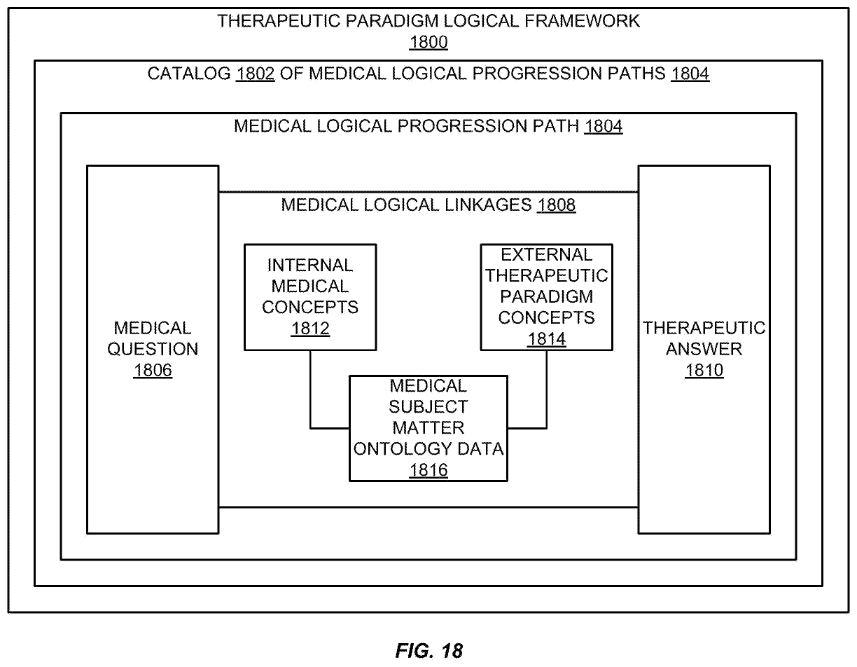

FIG. 18 shows an example therapeutic paradigm logical framework 1800. The therapeutic paradigm logical framework 1800 includes a catalog 1802 of medical logical progression paths 1804 from the medical question 1806 to respective therapeutic answers 1810.

Each of the medical logical progression paths 1804 can include one or more medical logical linkages 1808 from the medical question 1806 to a therapeutic path-specific answer 1810.

The medical logical linkages 1808 can include the internal medical concepts 1812 and external therapeutic paradigm concepts 1814 derived from a store of medical subject matter ontology data 1816. In some embodiments, the store of subject matter ontology data 1816 is contained in a knowledge cloud. In some embodiments, the knowledge cloud is the knowledge cloud 102 of FIGS. 1 and 2. In some embodiments, the subject matter ontology data 1816 is the subject matter ontology data 216 of FIG. 2. In some embodiments, the subject matter ontology data 1816 includes the subject matter ontology 300 of FIG. 3.

The method 1700 shown in FIG. 17 further includes selecting a likely medical information path from among the medical logical progression paths 1804 to a likely path-dependent medical information answer based at least in part upon the therapeutic intent of the user (block 1714). In some embodiments, selecting a likely medical information path from among the medical logical progression paths 1804 to a likely path-dependent medical information answer based at least in part upon the therapeutic intent of the user (block 1714 is performed by a critical thinking engine configured for this purpose. In some embodiments, the critical thinking engine is the critical thinking engine 108 of FIG. 1. The selection can also be based in part upon the sufficiency of medical diagnostic data to complete the medical logical linkages 1808. In some embodiments, selection can also be based in part upon the sufficiency of medical diagnostic data to complete the medical logical linkages 1808 can be performed by a critical thinking engine that is further configured for this purpose. In some embodiments, the critical thinking engine is the critical thinking engine 108 of FIG. 1. The medical diagnostic data can include user-specific medical diagnostic data. The selection can also be based in part upon treatment sub-intents including tactical constituents related to the therapeutic intent of the user by the store of medical subject matter ontology data 1816. In some embodiments, selection based in part upon treatment sub-intents including tactical constituents related to the therapeutic intent of the user by the store of medical subject matter ontology data 1816 can be performed by a critical thinking engine further configured for this purpose. In some embodiments, the critical thinking engine is the critical thinking engine 108 of FIG. 1. The selection can further occur after requesting additional medical diagnostic data from the user. An example of requesting additional medical diagnostic data from the user is shown in FIG. 4 on line 406 “I need some additional information in order to answer this question, was this an in-home glucose test or was it done by a lab or testing service”. In some embodiments, the process of selection after requesting additional medical diagnostic data from the user can be performed by a critical thinking engine further configured for this purpose. In some embodiments, the critical thinking engine is the critical thinking engine 108 of FIG. 1. In some embodiments, selecting a likely medical information path from among the medical logical progression paths 1804 to a likely path-dependent medical information answer based at least in part upon the therapeutic intent of the user (block 1714) is accomplished through one or more of Steps 5-6 as earlier discussed in the context of “Analyzing Conversational Context As Part of Conversational Analysis”.

The method 1700 involves answering the medical question by following the likely medical information path to the likely path-dependent medical information answer (block 1716). In some embodiments, answering the medical question by following the likely medical information path to the likely path-dependent medical information answer (block 1716) is performed by a critical thinking engine configured for this purpose. In some embodiments, the critical thinking engine is the critical thinking engine 108 of FIG. 1. In some embodiments, answering the medical question by following the likely medical information path to the likely path-dependent medical information answer (block 1716) is accomplished as in Step 7 as earlier discussed in the context of “Analyzing Conversational Context As Part of Conversational Analysis”.

The method 1700 can further include relating medical inference groups of the internal medical concepts. In some embodiments, relating medical inference groups of the internal medical concepts is performed by a critical thinking engine further configured for this purpose. In some embodiments, the critical thinking engine is the critical thinking engine 108 of FIG. 1. Relating medical inference groups of the internal medical concepts can be based at least in part on shared medical data entities for which each internal medical concept of a medical inference group of internal medical concepts describes a respective medical data attribute. In some embodiments, relating medical inference groups of the internal medical concepts based at least in part on shared medical data entities for which each internal medical concept of a medical inference group of internal medical concepts describes a respective medical data attribute can be performed by a critical thinking engine further configured for this purpose. In some embodiments, the critical thinking engine is the critical thinking engine 108 of FIG. 1.

In some embodiments, the method 1700 of FIG. 17 is implemented as a computer program product in a computer-readable medium.

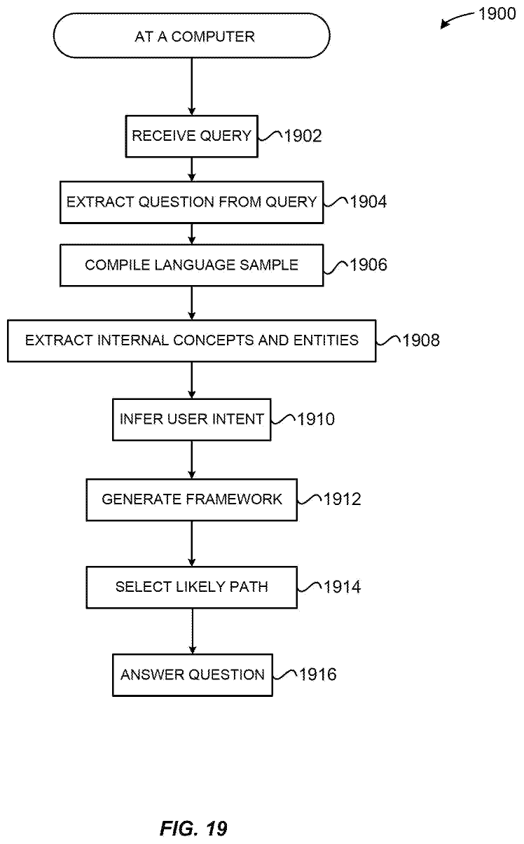

FIG. 19 shows a computer-implemented method 1900 for answering natural language questions posed by a user of a conversational interface of an artificial intelligence system. In some embodiments, the method 1900 is implemented on a cognitive intelligence platform. In some embodiments, the cognitive intelligence platform is the cognitive intelligence platform 102 as shown in FIG. 1. In some embodiments, the cognitive intelligence platform is implemented on the computing device 1400 shown in FIG. 14.

The method 1900 involves receiving a user-generated natural language query at an artificial intelligence-based conversation agent (block 1902). In some embodiments, receiving a user-generated natural language query from a conversational user interface at an artificial intelligence-based conversation cognitive agent (block 1902) is performed by a cognitive agent that is a part of the cognitive intelligence platform and is configured for this purpose. In some embodiments, the artificial intelligence-based conversation agent is the conversation agent 110 of FIG. 1. One example of a user-generated natural language query is “Is a blood sugar of 90 normal?” as shown in line 402 of FIG. 4. In some embodiments, the user interface is on the mobile device 104 of FIG. 1. In some embodiments, receiving a user-generated natural language query from a conversational user interface at an artificial intelligence-based conversation cognitive agent (block 1902) is Step 1 as earlier discussed in the context of “Analyzing Conversational Context As Part of Conversational Analysis”.

The method 1900 further includes extracting a question from a user of the conversational user interface from the user-generated natural language query (block 1904). In some embodiments, extracting a question from a user of the conversational user interface from the user-generated natural language query (block 1904) is performed by a critical thinking engine configured for this purpose. In some embodiments, the critical thinking engine is the critical thinking engine 108 of FIG. 1. In some embodiments, extracting a question from a user of the conversational user interface from the user-generated natural language query (block 1904) is accomplished through one or more of Steps 2-6 as earlier discussed in the context of “Analyzing Conversational Context As Part of Conversational Analysis”.

The method 1900 includes compiling a language sample (block 1906). In some embodiments, compiling a language sample (block 1906) is performed by a critical thinking engine configured for this purpose. In some embodiments, the critical thinking engine is the critical thinking engine 108 of FIG. 1. The language sample can include items of health-information-related-text derived from a health-related conversation between the artificial intelligence-based conversation cognitive agent and the user. In some embodiments compiling a language sample (block 1906) is accomplished through one or more of Steps 2-6 as earlier discussed in the context of “Analyzing Conversational Context As Part of Conversational Analysis”.

The method 1900 involves extracting internal concepts and entities from the language sample (block 1908). In some embodiments, extracting internal concepts and entities from the language sample (block 1908) is performed by a critical thinking engine configured for this purpose. In some embodiments, the critical thinking engine is the critical thinking engine 108 of FIG. 1. The internal concepts can include descriptions of attributes of the entities. In some embodiments, extracting internal concepts and entities from the language sample (block 1908) is accomplished through one or more of Steps 2-6 as earlier discussed in the context of “Analyzing Conversational Context As Part of Conversational Analysis”.

The method 1900 involves inferring an intent of the user from the internal concepts and the entities (block 1910). In some embodiments, inferring an intent of the user from the internal concepts and the entities (block 1910) is performed by a critical thinking engine configured for this purpose. In some embodiments, the critical thinking engine is the critical thinking engine 108 of FIG. 1. In some embodiments, inferring an intent of the user from the internal concepts and the entities (block 1910) is accomplished as in Step 2 as earlier discussed in the context of “Analyzing Conversational Context As Part of Conversational Analysis”.

The method 1900 includes generating a logical framework 2000 for interpreting of the question (block 1912). In some embodiments, generating a logical framework 2000 for interpreting of the question (block 1912) is performed by a critical thinking engine configured for this purpose. In some embodiments, the critical thinking engine is the critical thinking engine 108 of FIG. 1. In some embodiments, generating a logical framework 2000 for interpreting of the question (block 1912) is accomplished as in Step 5 as earlier discussed in the context of “Analyzing Conversational Context As Part of Conversational Analysis”.

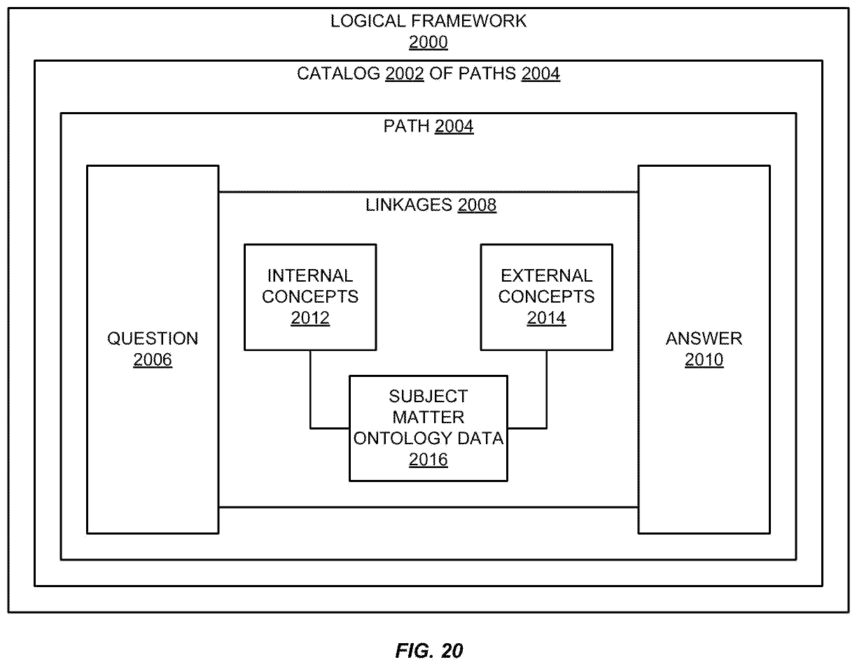

FIG. 20 shows an example logical framework 2000. The logical framework 2000 includes a catalog 2002 of paths 2004 from the question 2006 to respective answers 2010.

Each of the paths 2004 can include one or more linkages 2008 from the question 2006 to a path-specific answer 2010.

The linkages 2008 can include the internal concepts 2012 and external concepts 2014 derived from a store of subject matter ontology data 2016. In some embodiments, the store of subject matter ontology data 2016 is contained in a knowledge cloud. In some embodiments, the knowledge cloud is the knowledge cloud 102 of FIGS. 1 and 2. In some embodiments, the subject matter ontology data 2016 is the subject matter ontology data 216 of FIG. 2. In some embodiments, the subject matter ontology data 2016 includes the subject matter ontology 300 of FIG. 3.

The method 1900 shown in FIG. 19 further includes selecting a likely path from among the paths 2004 to a likely path-dependent answer based at least in part upon the intent of the user (block 1914). In some embodiments, selecting a likely path from among the paths 2004 to a likely path-dependent answer based at least in part upon the intent of the user (block 1914 is performed by a critical thinking engine configured for this purpose. In some embodiments, the critical thinking engine is the critical thinking engine 108 of FIG. 1. The selection can also be based in part upon the sufficiency of data to complete the linkages 2008. In some embodiments, selection can also be based in part upon the sufficiency of data to complete the linkages 2008 can be performed by a critical thinking engine that is further configured for this purpose. In some embodiments, the critical thinking engine is the critical thinking engine 108 of FIG. 1. The data can include user-specific data. The selection can also be based in part upon treatment sub-intents including tactical constituents related to the intent of the user by the store of subject matter ontology data 2016. In some embodiments, selection based in part upon treatment sub-intents including tactical constituents related to the intent of the user by the store of subject matter ontology data 2016 can be performed by a critical thinking engine further configured for this purpose. In some embodiments, the critical thinking engine is the critical thinking engine 108 of FIG. 1. The selection can further occur after requesting additional data from the user. An example of requesting additional data from the user is shown in FIG. 4 on line 406 “I need some additional information in order to answer this question, was this an in-home glucose test or was it done by a lab or testing service”. In some embodiments, the process of selection after requesting additional data from the user can be performed by a critical thinking engine further configured for this purpose. In some embodiments, the critical thinking engine is the critical thinking engine 108 of FIG. 1. In some embodiments, selecting a likely path from among the paths 2004 to a likely path-dependent answer based at least in part upon the intent of the user (block 1914) is accomplished through one or more of Steps 5-6 as earlier discussed in the context of “Analyzing Conversational Context As Part of Conversational Analysis”.

The method 1900 involves answering the question by following the likely path to the likely path-dependent answer (block 1916). In some embodiments, answering the question by following the likely path to the likely path-dependent answer (block 1916) is performed by a critical thinking engine configured for this purpose. In some embodiments, the critical thinking engine is the critical thinking engine 108 of FIG. 1. In some embodiments, answering the question by following the likely path to the likely path-dependent answer (block 1916) is accomplished as in Step 7 as earlier discussed in the context of “Analyzing Conversational Context As Part of Conversational Analysis”.

The method 1900 can further include relating inference groups of the internal concepts. In some embodiments, relating inference groups of the internal concepts is performed by a critical thinking engine further configured for this purpose. In some embodiments, the critical thinking engine is the critical thinking engine 108 of FIG. 1. Relating inference groups of the internal concepts can be based at least in part on shared entities for which each internal concept of an inference group of internal concepts describes a respective data attribute. In some embodiments, relating inference groups of the internal concepts based at least in part on shared entities for which each internal concept of an inference group of internal concepts describes a respective data attribute can be performed by a critical thinking engine further configured for this purpose. In some embodiments, the critical thinking engine is the critical thinking engine 108 of FIG. 1.

In some embodiments, the method 1900 of FIG. 19 is implemented as a computer program product in a computer-readable medium.

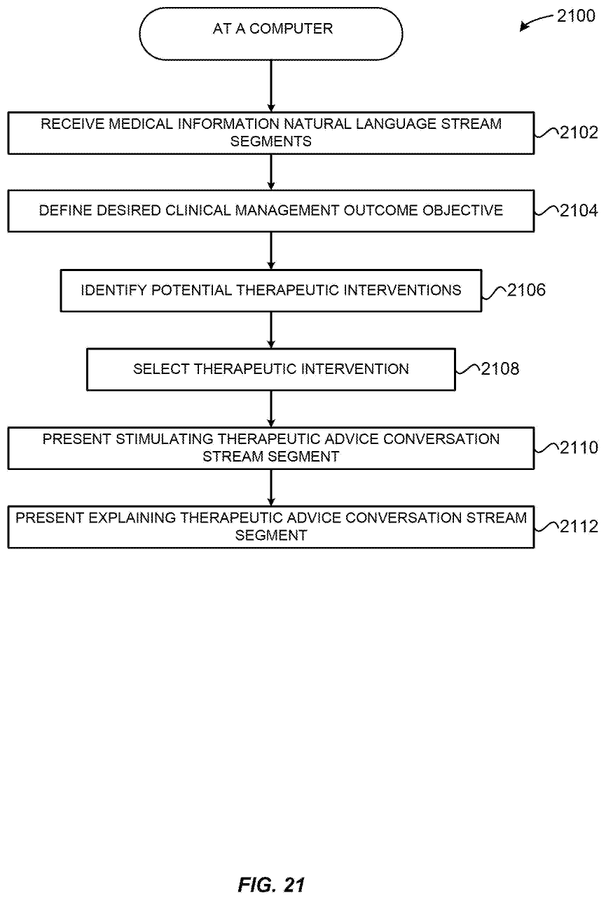

FIG. 21 shows a computer-implemented method 2100 for providing therapeutic medical action recommendations in response to a medical information natural language conversation stream. In some embodiments, the method 2100 is implemented as a computer program product in a non-transitory computer-readable medium. In some embodiments, the method 2100 of FIG. 21 is implemented as a system for providing therapeutic medical action recommendations in response to a medical information natural language conversation stream. The system can include a knowledge cloud, a critical thinking engine, and a cognitive agent. In some embodiments, the knowledge cloud is the knowledge cloud 102 of FIGS. 1 and 2. In some embodiments, the critical thinking engine is the critical thinking engine 108 of FIG. 1. In some embodiments, the cognitive agent is the cognitive agent 110 of FIG. 1.

In some embodiments, the method 2100 involves receiving segments of a medical information natural language conversation stream at an artificial intelligence-based health information conversation agent from a medical information conversation user interface (block 2102). In some embodiments the user interface is on the mobile device 104 of FIG. 1. In some embodiments, receiving segments of a medical information natural language conversation stream at an artificial intelligence-based health information conversation agent from a medical information conversation user interface (block 2102) is performed on a processor of a computer. In some embodiments, receiving segments of a medical information natural language conversation stream at an artificial intelligence-based health information conversation agent from a medical information conversation user interface (block 2102) is performed at a knowledge clout configured for this purpose. In some embodiments, receiving segments of a medical information natural language conversation stream at an artificial intelligence-based health information conversation agent from a medical information conversation user interface (block 2102) is Step 1 as earlier discussed in the context of “Analyzing Conversational Context As Part of Conversational Analysis”.

In some embodiments, the method 2100 further involves defining a desired clinical management outcome objective relevant to health management criteria and related health management data attributes of the user medical information profile in response to medical information content of a user medical information profile associated with the medical information natural language conversation stream (block 2104). In some embodiments, defining a desired clinical management outcome objective relevant to health management criteria and related health management data attributes of the user medical information profile in response to medical information content of a user medical information profile associated with the medical information natural language conversation stream (block 2104) is performed on a processor of a computer. In some embodiments, defining a desired clinical management outcome objective relevant to health management criteria and related health management data attributes of the user medical information profile in response to medical information content of a user medical information profile associated with the medical information natural language conversation stream (block 2104) is performed by a critical thinking engine configured for this purpose.

In some embodiments, defining a desired clinical management outcome objective relevant to health management criteria and related health management data attributes of the user medical information profile in response to medical information content of a user medical information profile associated with the medical information natural language conversation stream (block 2104) is accomplished through one or more of Steps 2-6 as earlier discussed in the context of “Analyzing Conversational Context As Part of Conversational Analysis”.

In some embodiments, the method 2100 further involves identifying a set of potential therapeutic interventions correlated to advancement of the clinical management outcome objective (block 2106). In some embodiments, identifying a set of potential therapeutic interventions correlated to advancement of the clinical management outcome objective (block 2106) is performed on a processor of a computer. In some embodiments, identifying a set of potential therapeutic interventions correlated to advancement of the clinical management outcome objective (block 2106) is performed by a critical thinking engine configured for this purpose. In some embodiments, identifying a set of potential therapeutic interventions correlated to advancement of the clinical management outcome objective (block 2106) is accomplished through one or more of Steps 2-6 as earlier discussed in the context of “Analyzing Conversational Context As Part of Conversational Analysis”.

In some embodiments, the method 2100 further involves selecting from among the set of potential therapeutic interventions correlated to advancement of the clinical management outcome objective a medical intervention likely to advance the clinical management outcome objective (block 2108). In some embodiments, selecting from among the set of potential therapeutic interventions correlated to advancement of the clinical management outcome objective a medical intervention likely to advance the clinical management outcome objective (block 2108) is based on a set of factors including the likelihood of patient compliance with the a recommendation for the a medical intervention and a statistical likelihood that the action will materially advance the clinical management outcome objective. In some embodiments, selecting from among the set of potential therapeutic interventions correlated to advancement of the clinical management outcome objective a medical intervention likely to advance the clinical management outcome objective (block 2108) is based on a set of factors comprising likelihood total expected cost expectation associated with the recommendation for the a medical intervention likely to advance the clinical management outcome objective. In some embodiments, selecting from among the set of potential therapeutic interventions correlated to advancement of the clinical management outcome objective a medical intervention likely to advance the clinical management outcome objective (block 2108) is performed on a processor of a computer. In some embodiments, selecting from among the set of potential therapeutic interventions correlated to advancement of the clinical management outcome objective a medical intervention likely to advance the clinical management outcome objective (block 2108) is performed by a critical thinking engine configured for this purpose. In some embodiments, selecting from among the set of potential therapeutic interventions correlated to advancement of the clinical management outcome objective a medical intervention likely to advance the clinical management outcome objective (block 2108) is accomplished through one or more of Steps 2-6 as earlier discussed in the context of “Analyzing Conversational Context As Part of Conversational Analysis”.

In some embodiments, the method 2100 further involves presenting in the medical information natural language conversation stream a therapeutic advice conversation stream segment designed to stimulate execution of the medical intervention likely to advance the clinical management outcome objective (block 2110). In some embodiments, the stimulation can be a motivation. In some embodiments, presenting in the medical information natural language conversation stream a therapeutic advice conversation stream segment designed to stimulate execution of the medical intervention likely to advance the clinical management outcome objective (block 2110) includes presenting to the user in the medical information natural language conversation stream a therapeutic advice conversation stream segment explaining a cost-benefit analysis comparing likely results of performance of the action likely to advance the clinical management outcome objective and likely results of non-performance of the action likely to advance the clinical management outcome objective. In some embodiments, presenting in the medical information natural language conversation stream a therapeutic advice conversation stream segment designed to stimulate execution of the medical intervention likely to advance the clinical management outcome objective (block 2110) includes presenting to the user in the medical information natural language conversation stream a conversation stream reinforcing the recommendation after expiration of a delay period. In some embodiments, presenting in the medical information natural language conversation stream a therapeutic advice conversation stream segment designed to stimulate execution of the medical intervention likely to advance the clinical management outcome objective (block 2110) includes presenting to the user in the medical information natural language conversation stream a therapeutic advice conversation stream segment explaining reasons for selection of the clinical management outcome objective. In some embodiments, presenting in the medical information natural language conversation stream a therapeutic advice conversation stream segment designed to stimulate execution of the medical intervention likely to advance the clinical management outcome objective (block 2110) includes notifying third party service providers of the clinical management outcome objective and the recommendation. In some embodiments, presenting in the medical information natural language conversation stream a therapeutic advice conversation stream segment designed to stimulate execution of the medical intervention likely to advance the clinical management outcome objective (block 2110) is performed on a processor of a computer. In some embodiments, presenting in the medical information natural language conversation stream a therapeutic advice conversation stream segment designed to stimulate execution of the medical intervention likely to advance the clinical management outcome objective (block 2110) is performed by a cognitive agent configured for this purpose. In some embodiments, presenting in the medical information natural language conversation stream a therapeutic advice conversation stream segment designed to stimulate execution of the medical intervention likely to advance the clinical management outcome objective (block 2110) is Steps 7 as earlier discussed in the context of “Analyzing Conversational Context As Part of Conversational Analysis”.

In some embodiments, the method 2100 further involves presenting to the user in the medical information natural language conversation stream a therapeutic advice conversation stream segment explaining a correlation between the medical intervention likely to advance the clinical management outcome objective and achievement of the clinical management outcome objective (block 2112). In some embodiments, presenting to the user in the medical information natural language conversation stream a therapeutic advice conversation stream segment explaining a correlation between the medical intervention likely to advance the clinical management outcome objective and achievement of the clinical management outcome objective (block 2112) is performed on a processor of a computer. In some embodiments, presenting to the user in the medical information natural language conversation stream a therapeutic advice conversation stream segment explaining a correlation between the medical intervention likely to advance the clinical management outcome objective and achievement of the clinical management outcome objective (block 2112) is performed by a critical thinking engine configured for this purpose. In some embodiments, presenting to the user in the medical information natural language conversation stream a therapeutic advice conversation stream segment explaining a correlation between the medical intervention likely to advance the clinical management outcome objective and achievement of the clinical management outcome objective (block 2112) is Steps 7 as earlier discussed in the context of “Analyzing Conversational Context As Part of Conversational Analysis”.

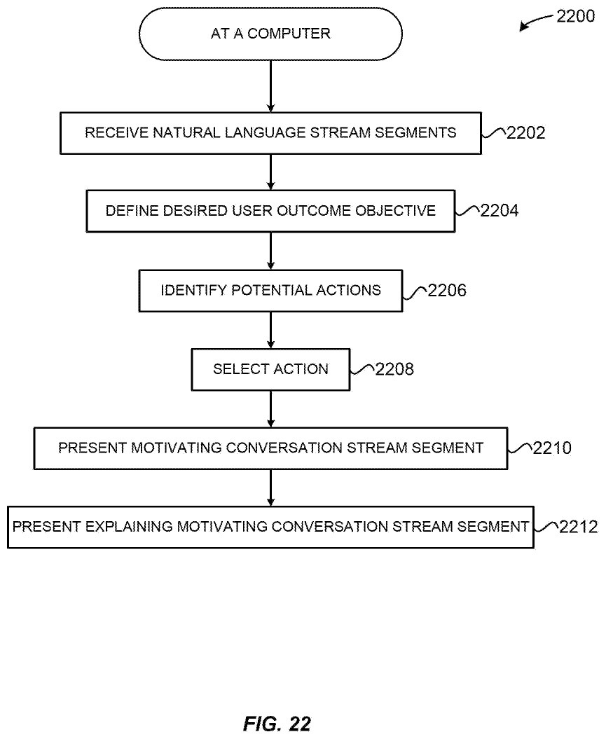

FIG. 22 shows a computer-implemented method 2200 for providing action recommendations in response to a natural language conversation stream. In some embodiments, the method 2200 is implemented as a computer program product in a non-transitory computer-readable medium. In some embodiments, the method 2200 of FIG. 22 is implemented as a system for providing action recommendations in response to a natural language conversation stream. The system can include a knowledge cloud, a critical thinking engine, and a cognitive agent. In some embodiments, the knowledge cloud is the knowledge cloud 102 of FIGS. 1 and 2. In some embodiments, the critical thinking engine is the critical thinking engine 108 of FIG. 1. In some embodiments, the cognitive agent is the cognitive agent 110 of FIG. 1.

In some embodiments, the method 2200 involves receiving segments of a natural language conversation stream at an artificial intelligence-based health information conversation agent from a conversation user interface (block 2202). In some embodiments the user interface is on the mobile device 104 of FIG. 1. In some embodiments, receiving segments of a natural language conversation stream at an artificial intelligence-based health information conversation agent from a conversation user interface (block 2202) is performed on a processor of a computer. In some embodiments, receiving segments of a natural language conversation stream at an artificial intelligence-based health information conversation agent from a conversation user interface (block 2202) is performed at a knowledge clout configured for this purpose. In some embodiments, receiving segments of a natural language conversation stream at an artificial intelligence-based health information conversation agent from a conversation user interface (block 2202) is Step 1 as earlier discussed in the context of “Analyzing Conversational Context As Part of Conversational Analysis”.

In some embodiments, the method 2200 further involves defining a desired user outcome objective relevant to health management criteria and related health management data attributes of the user profile in response to content of a user profile associated with the natural language conversation stream (block 2204). In some embodiments, defining a desired user outcome objective relevant to health management criteria and related health management data attributes of the user profile in response to content of a user profile associated with the natural language conversation stream (block 2204) is performed on a processor of a computer. In some embodiments, defining a desired user outcome objective relevant to health management criteria and related health management data attributes of the user profile in response to content of a user profile associated with the natural language conversation stream (block 2204) is performed by a critical thinking engine configured for this purpose.

In some embodiments, defining a desired user outcome objective relevant to health management criteria and related health management data attributes of the user profile in response to content of a user profile associated with the natural language conversation stream (block 2204) is accomplished through one or more of Steps 2-6 as earlier discussed in the context of “Analyzing Conversational Context As Part of Conversational Analysis”.

In some embodiments, the method 2200 further involves identifying a set of potential actions correlated to advancement of the user outcome objective (block 2206). In some embodiments, identifying a set of potential actions correlated to advancement of the user outcome objective (block 2206) is performed on a processor of a computer. In some embodiments, identifying a set of potential actions correlated to advancement of the user outcome objective (block 2206) is performed by a critical thinking engine configured for this purpose. In some embodiments, identifying a set of potential actions correlated to advancement of the user outcome objective (block 2206) is accomplished through one or more of Steps 2-6 as earlier discussed in the context of “Analyzing Conversational Context As Part of Conversational Analysis”.

In some embodiments, the method 2200 further involves selecting from among the set of potential actions correlated to advancement of the user outcome objective an action likely to advance the user outcome objective (block 2208). In some embodiments, selecting from among the set of potential actions correlated to advancement of the user outcome objective an action likely to advance the user outcome objective (block 2208) is based on a set of factors including the likelihood of patient compliance with the a recommendation for the an action and a statistical likelihood that the action will materially advance the user outcome objective. In some embodiments, selecting from among the set of potential actions correlated to advancement of the user outcome objective an action likely to advance the user outcome objective (block 2208) is based on a set of factors comprising likelihood total expected cost expectation associated with the recommendation for the an action likely to advance the user outcome objective. In some embodiments, selecting from among the set of potential actions correlated to advancement of the user outcome objective an action likely to advance the user outcome objective (block 2208) is performed on a processor of a computer. In some embodiments, selecting from among the set of potential actions correlated to advancement of the user outcome objective an action likely to advance the user outcome objective (block 2208) is performed by a critical thinking engine configured for this purpose. In some embodiments, selecting from among the set of potential actions correlated to advancement of the user outcome objective an action likely to advance the user outcome objective (block 2208) is accomplished through one or more of Steps 2-6 as earlier discussed in the context of “Analyzing Conversational Context As Part of Conversational Analysis”.

In some embodiments, the method 2200 further involves presenting in the natural language conversation stream a conversation stream segment designed to motivate performance of the action likely to advance the user outcome objective (block 2210). In some embodiments, presenting in the natural language conversation stream a conversation stream segment designed to motivate performance of the action likely to advance the user outcome objective (block 2210) includes presenting to the user in the natural language conversation stream a conversation stream segment explaining a cost-benefit analysis comparing likely results of performance of the action likely to advance the user outcome objective and likely results of non-performance of the action likely to advance the user outcome objective. In some embodiments, presenting in the natural language conversation stream a conversation stream segment designed to motivate performance of the action likely to advance the user outcome objective (block 2210) includes presenting to the user in the natural language conversation stream a conversation stream reinforcing the recommendation after expiration of a delay period. In some embodiments, presenting in the natural language conversation stream a conversation stream segment designed to motivate performance of the action likely to advance the user outcome objective (block 2210) includes presenting to the user in the natural language conversation stream a conversation stream segment explaining reasons for selection of the user outcome objective. In some embodiments, presenting in the natural language conversation stream a conversation stream segment designed to motivate performance of the action likely to advance the user outcome objective (block 2210) includes notifying third party service providers of the user outcome objective and the recommendation. In some embodiments, presenting in the natural language conversation stream a conversation stream segment designed to motivate performance of the action likely to advance the user outcome objective (block 2210) is performed on a processor of a computer. In some embodiments, presenting in the natural language conversation stream a conversation stream segment designed to motivate performance of the action likely to advance the user outcome objective (block 2210) is performed by a cognitive agent configured for this purpose. In some embodiments, presenting in the natural language conversation stream a conversation stream segment designed to motivate performance of the action likely to advance the user outcome objective (block 2210) is Steps 7 as earlier discussed in the context of “Analyzing Conversational Context As Part of Conversational Analysis”.

In some embodiments, the method 2200 further involves presenting to the user in the natural language conversation stream a conversation stream segment explaining a correlation between the action likely to advance the user outcome objective and achievement of the user outcome objective (block 2212). In some embodiments, presenting to the user in the natural language conversation stream a conversation stream segment explaining a correlation between the action likely to advance the user outcome objective and achievement of the user outcome objective (block 2212) is performed on a processor of a computer. In some embodiments, presenting to the user in the natural language conversation stream a conversation stream segment explaining a correlation between the action likely to advance the user outcome objective and achievement of the user outcome objective (block 2212) is performed by a critical thinking engine configured for this purpose. In some embodiments, presenting to the user in the natural language conversation stream a conversation stream segment explaining a correlation between the action likely to advance the user outcome objective and achievement of the user outcome objective (block 2212) is Steps 7 as earlier discussed in the context of “Analyzing Conversational Context As Part of Conversational Analysis”.

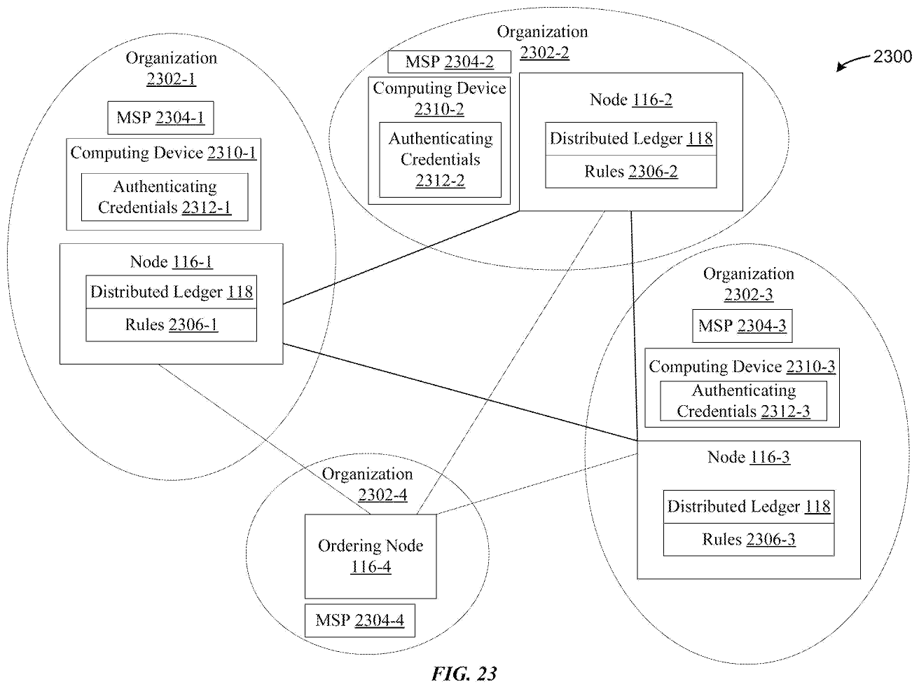

FIG. 23 shows distributed ledger fabric network 2300 of nodes 116 each maintaining a copy of a distributed ledger 118 to manage knowledge in a healthcare ecosystem, in accordance with various embodiments. The distributed ledger fabric network 2300 is divided into different organizations 2302 that may include one or more nodes 116 associated with that particular organization. An organization 2302 may refer to a security domain, unit of identity, and/or authentication information. Each organization 2302 may include one or more nodes 116 that are associated with a particular entity. For example, one organization 2302-1 may be associated with one or more patient entities that are registered as one or more nodes 116-1, another organization 2302-2 may be associated with one or more medical personnel entities that are registered as one or more nodes 116-2, another organization 2302-3 may be associated with one or more medical facility entities that are registered as one or more nodes 116-3, and so on for any suitable entities (e.g., insurance providers, government agencies, professional associations, etc.) in a healthcare ecosystem.

In some embodiments, the organizations 2302 may be associated with a combination of entities. For example, the entities that are associated with a hospital may be grouped into an organization 2302. The organization 2302 may be associated with a medical facility entity for the hospital, one or more medical personnel entities for the physicians, nurses, staff, etc., and/or one or more patient entities for each patient of the hospital.

Other organizations 2302 may be associated with nodes 116 representing services provided by the distributed ledger fabric network 2300. For example, organization 2302-4 is associated with an ordering node 116-4 that ensures that the one or more rules implemented by each of the nodes 116-1, 116-2, and/or 116-3 involved in a transaction are satisfied and/or there is consensus among the nodes 116-1, 116-2, and/or 116-3 prior to approving performance of the transaction and addition of a record of the transaction into the distributed ledger 118. Using the ordering node 116-4 enhances consistency and security of the distributed ledger 118 by controlling what is allowed to be added to the distributed ledger 118.

Each node 116 may implement various rules 2306 which may be accessed by the cognitive intelligence platform 102, installed and/or added to the distributed ledger 118, and/or the like. In some embodiments, the rules 2306 may be included in each respective copy of the distributed ledger 118 that is distributed between the various nodes 116. In some embodiments, a distributed ledger 118 on one node (e.g., 116-1) may have a first subset of the rules 2306 installed and another node (e.g., 116-2) may have a second subset of the rules 2306 installed in its copy of the distributed ledger 118 where at least one rule in the first subset is different than a rule in the second subset. The rules 2306 may be implemented as computer executable instructions (e.g., software modules).

The rules 2306 may be self-executing at certain frequencies. For example, after a period of time expires, a rule 2306 may determine whether certain information (e.g., authorization information) of an entity (e.g., medical personnel entity) registered as a node 116 needs to be updated in the distributed ledger 118 and provide a notification to a computing device 2310 used by that entity. In another example, a rule 2306 may determine that content that is stored on the distributed ledger 118 needs to be verified and/or updated after a monitored period of time expires since the content was last verified, updated, or generated. In other embodiments, the rules 2306 may be self-executing based on certain conditions occurring. For example, when authorization information of an entity expires in the distributed ledger 118, the rule 2306 may trigger a notification to be sent to the computing device 2310 of that entity. In other instances, the rules 2306-2 may be triggered when a request to perform a transaction on the distributed ledger 118 is received.

In general, the rules 2306 may be used to govern the content allowed to be stored on the distributed ledger 118, to control who is permitted to store content on the distributed ledger 118, to control who is permitted to update content on the distributed ledger 118, to control who is permitted to verify the source of the content, and/or to control who has access to the content, among other things. The rules 2306 may specify when updates to the distributed ledger 118 are to be provided. The rules 2306 may be analytics-based in that they monitor states or conditions of information in the distributed ledger 118, the computing devices 2310, and/or the nodes 116, and determine when distributed ledger 118 updates should be provided. For example, the rules 2306 may specify that updates to the distributed ledger 118 are to be provided based on any combination of geo-fencing (e.g., geolocation of the computing devices 2310 associated with particular nodes 116), authorization information of medical personnel being valid, time frames for verifying content, and/or the like.

Each organization 2302 may include a computing device 2310 used by an entity associated with that organization 2302. The computing device 2310 may include one or more memories, processors, and/or network interfaces. The computing device 2310 may be similar to any computing device described with respect to FIG. 14. The user may use the computing device 2310 to send requests to perform transactions using the distributed ledger 118 to the cognitive intelligence platform 102 that may include the nodes 116.

Each organization 2302 may include a membership service provider (MSP) 2304 that is responsible for issuing identities and authentication information 2312 to computing devices 2310 associated with entities. As described herein, when a computing device 2308 requests to perform a transaction, such as registering as a node 116 on the distributed ledger fabric network 2300, the computing device 2310 may provide certain information pertaining to the entity. For example, for a medical personnel entity the information may include at least an identity of the medical personnel entity, authorization information (e.g., NPI number, medical license number, etc.), a date the authorization information was last updated, an address of a place of work of the medical personnel entity, gender, race, and/or the like. For a patient entity, the information may include at least the patient's identity, social security number, driver's license number, address, medical records, allergies, medicine allergies, familial medical history, and/or the like. The information may be encrypted and securely stored on the distributed ledger 118 such that only appropriate entities can view the information of another entity.

The nodes 116 may communicate with each other to determine if a consensus is reached as to whether to allow the transaction to be performed. Further, one or more of the rules 2306 may be applied to determine whether to allow the transaction to be performed. When the consensus protocol and/or the rules 2306 are satisfied, the ordering node 116 may order the transaction to be performed and a record of the transaction is added to the distributed ledger 118.

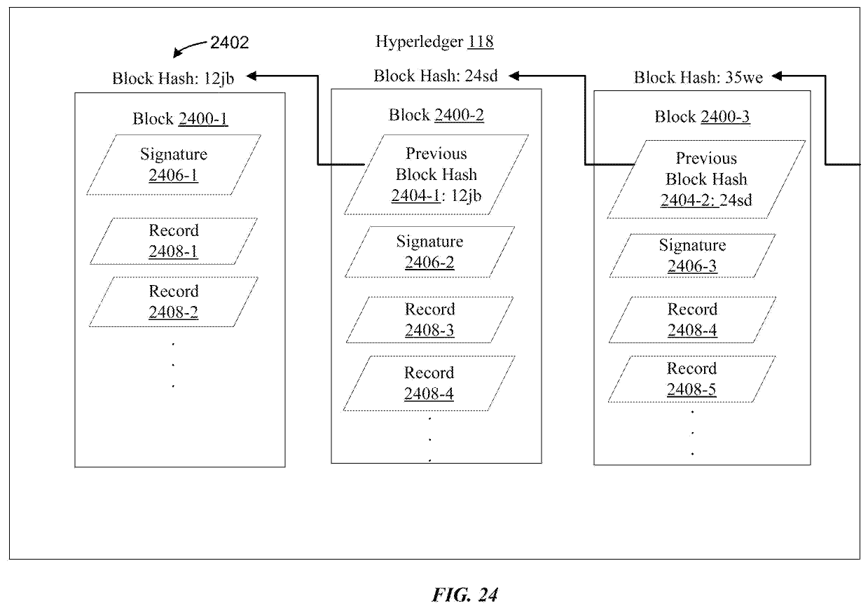

FIG. 24 shows an example distributed ledger 118, in accordance with various embodiments. As depicted, the distributed ledger 118 includes three blocks 2400-1, 2400-2, and 2400-3. Each of the blocks is cryptographically linked to a previous block. Each block 2400 includes a block hash 2402 for that block that is determined using a suitable hash function. For example, block 2400-1 has block hash 2402 “12jb”, block 2400-2 has block hash 2402 “24sd”, and block 2400-3 has block hash 2402 “35we”. The blocks 2400 are cryptographically linked together by including a block header with the block hash of the previous block. For example, block 2400-2 includes a block header including previous block hash 2404-1 with value “12jb”, which is the value of the previous block 2400-1 in the distributed ledger 118, and block 2400-3 includes a block header including previous block hash 2404-2 with value “24sd”, which is the value of the previous block 2400-2.

Each block 2400 includes a signature 2406 and one or more records 2408 (e.g., records of transactions). The signature 2406 may be the identity of the entity that requested the transaction to be performed. If there are multiple transactions stored as records 2408 on a block 2400 and different entities are associated with those transactions, there may be different signatures 2406 associated with the different entities stored on the blocks 2400. The signatures may be used to prove that the entity is the source for a transaction (e.g., generated and stored certain content). In some embodiments, a block 2400 storing records 24808 of transactions may be added to the distributed ledger 118 after it is determined that the one or more rules 2306 and/or consensus between the nodes 116 are satisfied. The transactions in a given request may be grouped and added as a block 2400 to the distributed ledger 118, or different transactions from different requests may be grouped and added as a block 2400 if the transactions are related or involve particular nodes 116.

In some embodiments, the transactions relating to registering an entity may store identifying information pertaining to an entity, such as an identity, address, social security number, driver's license number, and/or the like. Records 2408 of these transactions may store authorization information, such as license numbers (NPIs) for physicians, license numbers for pharmacists, license numbers for pharmacies to dispense medicine, and/or the like.

The records 2408 of transactions relating to storing content on the distributed ledger 118 may store text, videos, images, etc. generated by an entity. For example, the content may relate to healthcare and include evidence-based guidelines, knowledge representation, clinical studies, clinical processes, clinical techniques, care plans, and/or the like. The records 2408 of transactions may store various information for each content that is added to the distributed ledger 118. The information may include a title, a document type, an author, authorization information (e.g., medical license number, medical degree, etc.) of the author, an upload timestamp, a last update timestamp, content, an indicator of whether the content has been verified by one or more other licensed professionals, an identity of the one or more other licensed professionals, authorization information of the one or more other licensed professionals, and/or the like.

The records 2408 of transactions relating to providing content on the distributed ledger 118 may store an incremented counter value for a number of views of the content, a timestamp of the viewing of the content, as well as any information pertaining to the requesting entity. For example, the distributed ledger 118 may determine whether the requesting entity is associated with an authorized credential and increment a counter value that indicates the content was viewed by another entity having an authorized credential and store the timestamp that the content was provided to a computing device associated with the requesting entity. Other counters may be used, such as a counter for how many medical personnel entities having valid authorized credentials verified the content is accurate and good medical practice. Content associated with a higher number of views and/or verifications by entities having authorization information may be more trustworthy and selected to be provided by the rules when a user requests content.

In this regard, a ranking technique may be used by the rules to identify the content that is associated with a highest number of total views by the entities (e.g., patients, medical personnel), a highest number of total views by medical personnel entities having valid authorization information, a highest number of total verifications by medical personnel entities having valid authorization information, and/or the like. Providing the content that is viewed by more medical personnel over time than other content may enable the user to obtain more trustworthy content faster. As a result, processing, memory, and/or network resources may be saved by providing more trustworthy content using the distributed ledger 118 because the user may not perform multiple searches to find other content if initially provided with the most trustworthy content.

The records 2408 of transactions relating to storing updated content on the distributed ledger 118 may store the updated content that may include additional content in conjunction with original content. The additional content may include text documents, videos, images, etc. generated by an entity. The entity may be the same entity that generated the original content or may be a different entity than the entity that stored the original content. The updated content may relate to healthcare and include evidence-based guidelines, knowledge representation, clinical studies, clinical processes, clinical techniques, care plans, and/or the like. The rules 2306 may ensure that least a portion of the additional content is new relative to other content stored in the distributed ledger 118. The records 2408 of transactions may store various information for each updated content that is added to the distributed ledger 118. The information may include a title, a document type, an author, authorization information (e.g., medical license number, medical degree, etc.) of the author, an upload timestamp, a last update timestamp, updated content (e.g., original content and additional content), an indicator of whether the content has been verified by one or more other licensed professionals, an identity of the one or more other licensed professionals, authorization information of the one or more other licensed professionals, and/or the like.

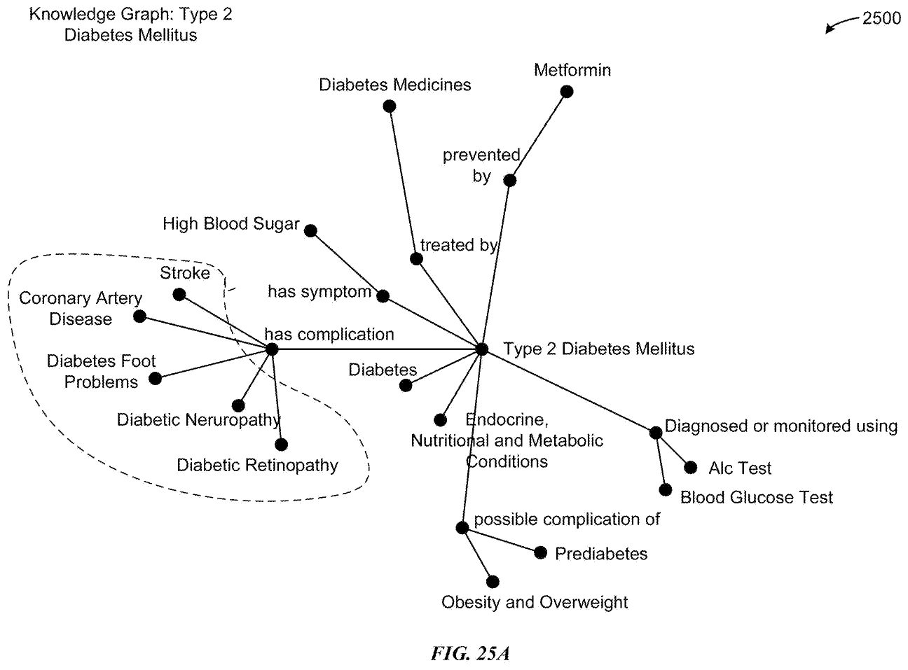

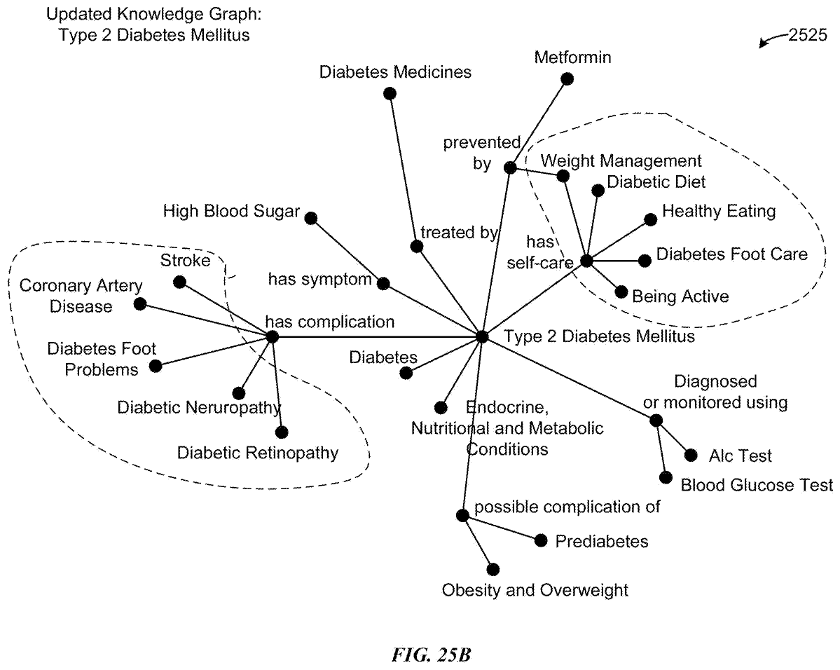

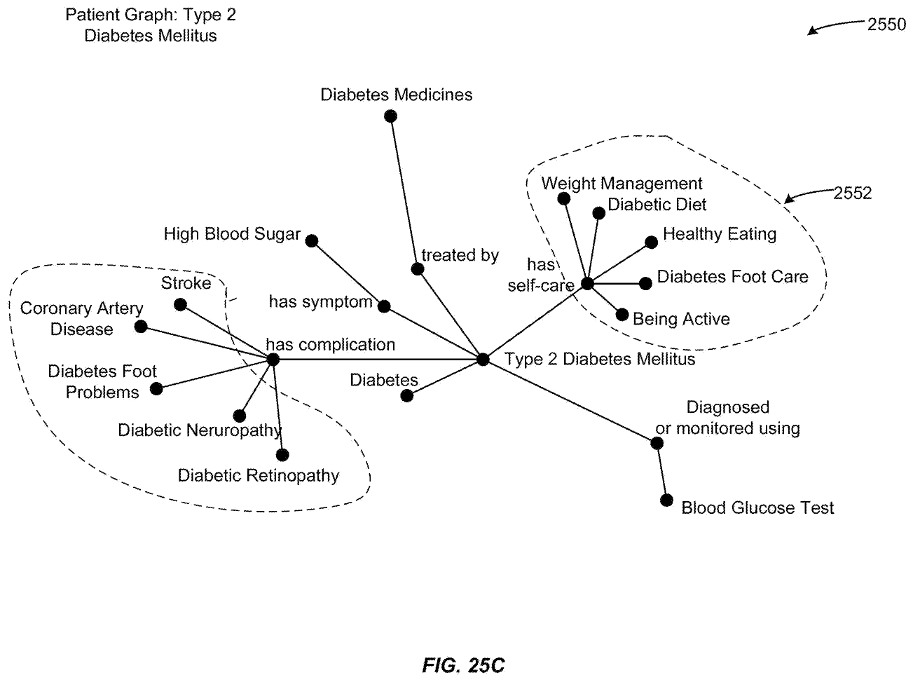

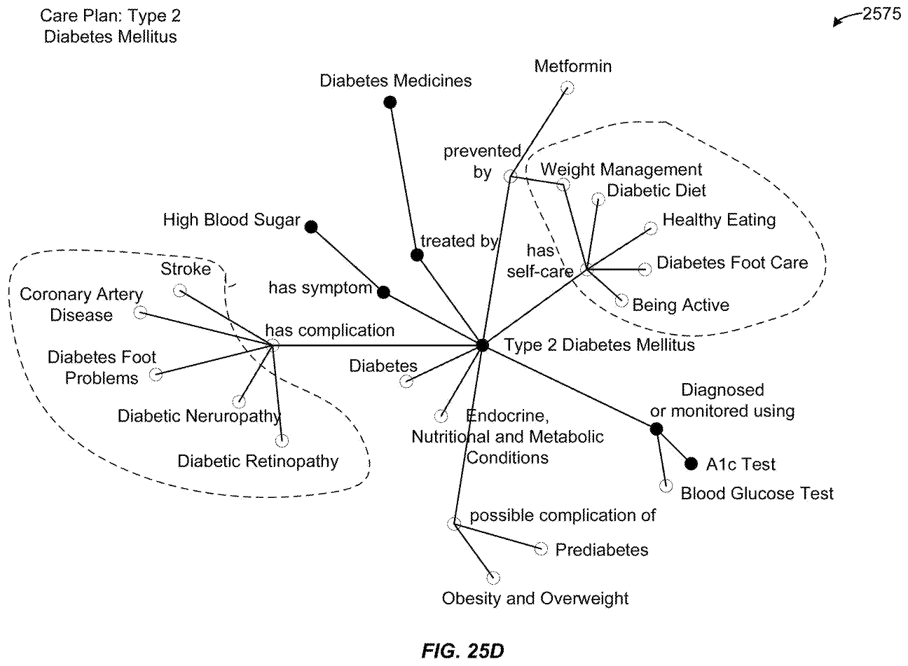

FIGS. 25A-25D show examples for using a knowledge graph 2500, an updated knowledge graph 2525, and/or a patient graph 2550 to generate a care plan (care plan 2575 may be a representation of at least a portion of the care plan). In particular, FIG. 25A shows a cognitive map or “knowledge graph” 2500 at a first knowledge stage, in accordance with various embodiments. The knowledge graph 2500 may be a representation of knowledge pertaining to Type 2 diabetes at a first knowledge stage including original content. The original content may include nodes in the knowledge graph 2500 that represent a health artifact or relationship for a particular patient of a medical personnel entity. The nodes may be generated from direct interrogation or indirect interactions with the user (by way of the user device 104). The original content including the nodes may include symptoms (High Blood Sugar), possible complications (Prediabetes, Obesity and Overweight), actual complications (e.g., Stroke, Coronary Artery Disease, Diabetes Foot Problems, Diabetic Neuropathy, Diabetic Retinopathy), how the medical condition is diagnosed or monitored (e.g., A1c Test, Blood Glucose Test), how the medical condition is treated (e.g., Diabetes Medicines), how the medical condition is prevented (e.g., Metformin), Diabetes, Endocrine, Nutritional, and Metabolic Conditions, and so forth.

A medical personnel entity may use a computing device 2310 to transmit a request to store the knowledge graph 2500 in the hyperledger 118. The hyperledger 118 may execute one or more rules to (i) determine that the user has proper authenticating credentials, (ii) determine that the user has valid authorizing credentials (e.g., medical degree from an accredited school, valid medical license, etc.), (iii) determine that the original content in the knowledge graph 2500 is not disclosed by other content stored in the hyperledger 118, and so forth. Further, the nodes may use a consensus protocol to determine whether to allow the transaction to be performed. If the one or more rules and/or the consensus protocol are satisfied, the knowledge graph 2500 may be stored in the hyperledger 118.

The medical personnel entity may input additional data that may be stored as part of an updated knowledge graph 2525 as shown in FIG. 25B. The knowledge graph 2500 evolved to the updated second knowledge graph 2525 based on the addition of additional content in the nodes in dotted area 2552 that pertain to anew self-care section for Type 2 Diabetes. The medical personnel entity may determine to add the new self-care section to the original content including the nodes previously described with reference to FIG. 25A. The self-care section may include nodes for Weight Management, Diabetic Diet, Healthy Eating, Diabetes Foot Care, and Being Active. Each node may include specific details pertaining to the respective topic that instruct the patient how to take care of themselves to reduce symptoms and/or overcome the medical condition represented in the knowledge graph 2500 (which may also be represented in the updated knowledge graph 2525).

It should be noted that the additional content including the new self-care section may be unique for treating Type 2 Diabetes for a patient relative to other content that relates to treating Type 2 Diabetes. Accordingly, the medical personnel entity may use a computing device 2310 to transmit a transaction request to store the knowledge graph 2525 on the hyperledger 118. The hyperledger may execute the one or more rules to (i) determine that the user has proper authenticating credentials, (ii) determine that the user has valid authorizing credentials (e.g., medical degree from an accredited school, valid medical license, etc.), (iii) determine that at least a portion of the additional content in the knowledge graph 2525 is not disclosed by other content stored in the hyperledger 118, and so forth. Further, the nodes may use a consensus protocol to determine whether to allow the transaction to be performed. If the one or more rules and/or the consensus protocol are satisfied, the knowledge graph 2525 may be stored in the hyperledger 118. A timestamp may be stored with the updated content each time updated content is stored on the hyperledger 118 to show a time series evolution of the content since the original content was first stored on the hyperledger 118 to a current state of the updated content.

The new self-care section may prove to provide great results for treating Type 2 Diabetes. Other medical personnel entities that are associated with nodes in the distributed hyperledger fabric network 2300 may request access to the knowledge graph 2525 stored in the hyperledger 118 using computing devices 2310. If the knowledge graph 2525 is set to public, the hyperledger 118 may provide the knowledge graph 2550 to the requesting entities. If the knowledge graph 2525 is set to private, the hyperledger 188 may provide the knowledge graph 2550 upon the requesting entity purchasing an access right to the knowledge graph 2550, or in various other scenarios.

FIG. 25C depicts the patient graph 2550 for a particular user having the condition Type 2 Diabetes Mellitus. The patient graph 2550 may also include an engagement profile as metadata that stores interactions of the patient with the various health artifacts presented in a care plan for the user. The interactions may be used to track a level of compliance with the care plan for the user. In some embodiments, the health artifacts represented by the nodes may be added to the patient graph as the patient interacts with the health artifacts. In some embodiments, the health artifacts may be added to the patient graph 2000 if the patient interacts with the health artifact to a threshold level.

As depicted, the patient graph 2550 includes a subset of the superset of health artifacts included in the knowledge graph 2500. For example, the patient graph 2550 includes a node representing a “Blood Glucose Test” health artifact that the patient performed. Various information (e.g., result, timestamp, etc.) pertaining to the blood glucose test may be associated with the node. However, the patient graph 2550 does not include a node representing the “A1c” health artifact that is included in the knowledge graph 2500 (and/or the updated knowledge graph 2525) because the patient has not interacted with that health artifact yet. In other words the patient has not performed the A1c test yet.

Other nodes representing health artifacts that are included in the knowledge graph 2500 (and/or the updated knowledge graph 2525) and not in the patient graph 2550 (e.g., due to the patient not interacting with those health artifacts yet) are a node representing “Endocrine, Nutritional and Metabolic Conditions”, a node representing “possible complication of” connected to nodes representing “Prediabetes” and “Obesity and Overweight”, and a node representing “prevented by” connected to a node representing “Metformin”.

To generate the care plan (a representation of at least a portion of the care plan is shown as care plan 2575, as is depicted in FIG. 25D), the cognitive intelligence platform 102 (e.g., the autonomous multipurpose application, the critical thinking engine 108, and/or the knowledge cloud 106) may compare the patient graph 2550 with the knowledge graph 2500 (and/or the updated knowledge graph 2525). Comparing the patient graph 2000 with the knowledge graph 2500 (and/or the updated knowledge graph 2525) may include projecting the patient graph 2550 onto the knowledge graph 2500 (and/or the updated knowledge graph 2525). In some embodiments, projecting the patient graph may include overlaying the patient graph 2575 on the knowledge graph 2500 (and/or the updated knowledge graph 2525), and/or plotting the patient graph 2575 in a same space as the knowledge graph 2500 (and/or the updated knowledge graph 2550). Based on the comparing, the cognitive intelligence platform 102 may select a subset of the superset of health artifacts in the knowledge graph 2500 (and/or in the updated knowledge graph 2525). The selecting may be based on identifying nodes representing health artifacts that are included in the knowledge graph 2500 (and/or the updated knowledge graph 2525) and not the patient graph 2550, and/or on areas of the condition the patient selected to manage.

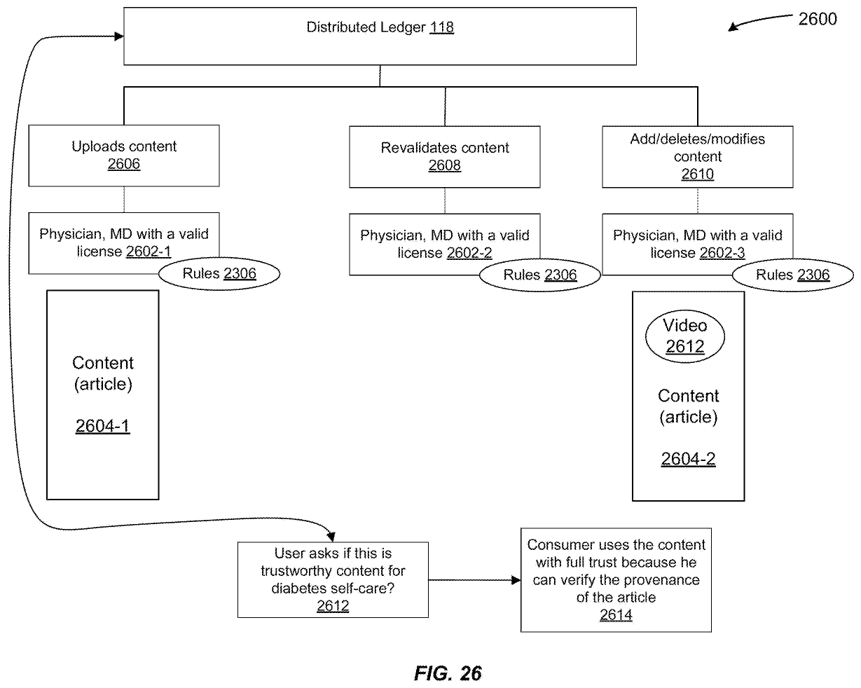

FIG. 26 shows a general process 2600 for performing transaction requests on a distributed ledger 118 by various nodes 116 in a healthcare ecosystem, in accordance with various embodiments. One or more rules 2306 may be used to determine when to allow transaction requests to be performed on the distributed ledger 118. The rules 2306 may be computer instructions executable by one or more processors of a node 116 (2602-1, 2602-2, 2602-3) representing an entity. The rules 2306 may be installed in the distributed ledger 118 and may specify scenarios when updates to the distributed ledger 118 based on analytics and/or when to allow transactions to be performed on the distributed ledger 118.

In one scenario, the rules 2306 may specify that the authorization information of a node 2602-1 representing a medical personnel entity has to be updated every X period of time in the distributed ledger 118. For example, a physician's 2502 medical license has to be updated every 3 years, and a pharmacist's 2504 license has to be updated every 5 years. The rules 2306 may analyze the information pertaining to the medical personnel 2602-1 in the records 2408 of transactions stored in the distributed ledger 118 and may determine that the period of time for updating the authorization information has expired or is about to expire. As a result, the node executing the rules 2306 may cause a notification to be presented on the computing device 2310 used by the medical personnel entity that instructs the medical personnel entity to update their authorization information. The updated authorization information may be stored on the distributed ledger 118.

In one embodiment, the rules 2306 may specify that the distributed ledger 118 is updated when a transaction, such as a request to upload (2606) content 2604-1, is received at a node 2602-1 associated with a physician, for example, and various checks made by the rules 2306 are satisfied and/or a consensus protocol used by the nodes 116 is satisfied. The content 2604-1 may be an article pertaining to healthcare that includes text and/or images. For example, the content 2604-1 may describe aspects of the knowledge graph 2500 for Type 2 Diabetes. The rules 2306 may determine (i) whether the physician requesting to store the content 2604-1 has valid authentication information (e.g., username, password, unique identifier), (ii) whether the physician requesting to store the content 2604-1 has valid authorization information (e.g., medical degree, medical license, etc.), and/or (iii) whether at least a portion of the content 2604-1 is new relative to other content in the distributed ledger 118 pertaining to the same topic. If the rules 2306 are satisfied and/or a consensus protocol used by the nodes 116 is satisfied, the content 2604-1 may be stored on the distributed ledger 118. In some embodiments, the consensus protocol may be satisfied when one or more of the rules 2306 are satisfied.

In one embodiment, the rules 2306 may specify that the content 2604-1 stored on the distributed ledger 118 be updated every certain period of time by the physician that uploaded the content 2604-1 or by another physician with valid authorization information. The rules 2306 may determine that a certain amount of time has elapsed since the content 2604-1 was generated, uploaded, and/or last verified, and may cause a notification to be transmitted to the computing device 2310 of an appropriate physician to verify the content 2604-1 is still valid.

Accordingly, at 2608, a computing device associated with the appropriate physician may transmit a request to review the content 2604-1 and verify the content 2604-1 is still valid medical practice/advice/care plan. The node 2602-2 may be associated with the same physician that uploaded the content 2604-1 or may be associated with another physician. The rules may determine (i) whether the physician requesting to verify the content 2604-1 has valid authentication information (e.g., username, password, unique identifier), and/or (ii) whether the physician requesting to verify the content 2604-1 has valid authorization information (e.g., medical degree, medical license, etc.). If the rules 2306 are satisfied and/or the consensus protocol is satisfied, the distributed ledger 118 may provide the content 2604-1 to the computing device associated with the requesting physician and the requesting physician can review the content 2604-1 and provide an indication to the distributed ledger, via the computing device, that the content 2604-1 is verified. In such an instance, the distributed ledger 118 may update a last verified timestamp. If the physician reviews the content 2604-1 and determines that information in the content 2604-1 is outdated or no longer valid, the physician may provide, via the computing device, an indication that the content 2604-1 is not verified. The distributed ledger 118 may prevent the content 2604-1 from being distributed upon further requests.

In one embodiment, the rules 2306 may specify that the distributed ledger 118 is updated when a transaction, such as a request to add, delete, or modify (2610) content 2604-1, is received at a node 2602-3 associated with a physician, for example, and various checks made by the rules 2306 are satisfied and/or a consensus protocol used by the nodes 116 is satisfied. The node 2602-3 may be associated with the same physician that initially uploaded the content 2604-1 or may be associated with another physician.

When the request is to add content, the same description provided for 2606 above may apply. When the request is to delete content, the rules 2306 may determine (i) whether the physician requesting to delete the content 2604-1 has valid authentication information (e.g., username, password, unique identifier), and/or (ii) whether the physician requesting to delete the content 2604-1 has valid authorization information (e.g., medical degree, medical license, etc.). If the physician requesting deletion of the content 2604-1 is not the same physician that generated and uploaded the content 2604-1, then the consensus protocol may not be satisfied unless the node 2602-1 associated with the physician that uploaded the content 2604-1 agrees to allow the content 2604-1 to be deleted.

If the request is to modify content 2604-1 stored on the distributed ledger 118, then the physician may have added additional content (e.g., a video 2612) to the original content to create updated content 2604-2. The additional content may explain different steps of a care plan. For example, the video 2612 in the updated content 2604-2 may present new steps of the self-care section for the knowledge graph 2550 in FIG. 25B. The rules 2306 may determine (i) whether the physician requesting to store the updated content 2604-2 has valid authentication information (e.g., username, password, unique identifier), (ii) whether the physician requesting to store the updated content 2604-2 has valid authorization information (e.g., medical degree, medical license, etc.), and/or (iii) whether at least a portion of the updated content 2604-2 is new relative to other content in the distributed ledger 118 pertaining to the same topic. In this example, metadata of the video 2612 may be used, the audio in the video may be translated to text, and/or object character recognition may be used to determine whether the video 2612 includes new content. If the rules 2306 are satisfied and/or a consensus protocol used by the nodes 116 is satisfied, the updated content 2604-2 may be stored on the distributed ledger 118. In some embodiments, the consensus protocol may be satisfied when one or more of the rules 2306 are satisfied.

In some embodiments, a user may be associated with a node 116 in the distributed ledger fabric network 2300. Accordingly, the user may provide authentication information that is used with a software application executing on a computing device to access the cognitive intelligence platform 102 to submit transaction requests. In some embodiments, the user may obtain the content 2604-2. For example, the physician that generated the updated content 2604-2 may provide the updated content 2604-2 to the user, or the user may submit a transaction request to search the distributed ledger 118 for content related to Type 2 Diabetes treatment. The distributed ledger 118 may provide the updated content 2604-2 that includes the new self-care section. The user can query (2612) whether the updated content 2604-2 is trustworthy content for diabetes self-care. In some embodiments, the distributed ledger 118 may provide the provenance of the content 2604-2 by presenting an identity of the physician that generated the updated content 2604-2, the authorization information (e.g., medical degree, medical license, etc.) of the physician that generated the updated content 2604-2, a date the updated content 2604-2 was last verified, and/or the like. Based on verification from the distributed ledger 118, the user (consumer) may use (2614) the updated content 2604-2 with full trust because he can verify the provenance of the updated content 2604-2.

In some embodiments, the user may submit a question asking whether the content 2604-2 is trustworthy content in natural language to the cognitive intelligence platform 102. In some embodiments, the cognitive intelligence platform is the cognitive intelligence platform 102 as shown in FIG. 1. In some embodiments, the cognitive intelligence platform is implemented on the computing device 1400 shown in FIG. 14. The cognitive intelligence platform 102 may receive the question at the cognitive agent 110 and may process the question using the critical thinking engine 108, natural language database 122, knowledge cloud 106, and/or conversation orchestrator 124 using any of the methods disclosed herein. The cognitive intelligence platform 102 may determine, using the distributed ledger 118, whether the content 2604-2 is trustworthy content by verifying that the content 2604-2 was generated by a medical personnel entity having valid authorization information, and/or the content 2604-2 has been updated/verified within a certain time period. If the content 2604-2 is determined to be trustworthy, the cognitive intelligence platform 102 may cause an indication to be presented on the computing device of the user device 104. The indication may be text confirming the content 2604-2 is trustworthy and/or a visual representation (e.g., a thumbs up, check mark, etc.).

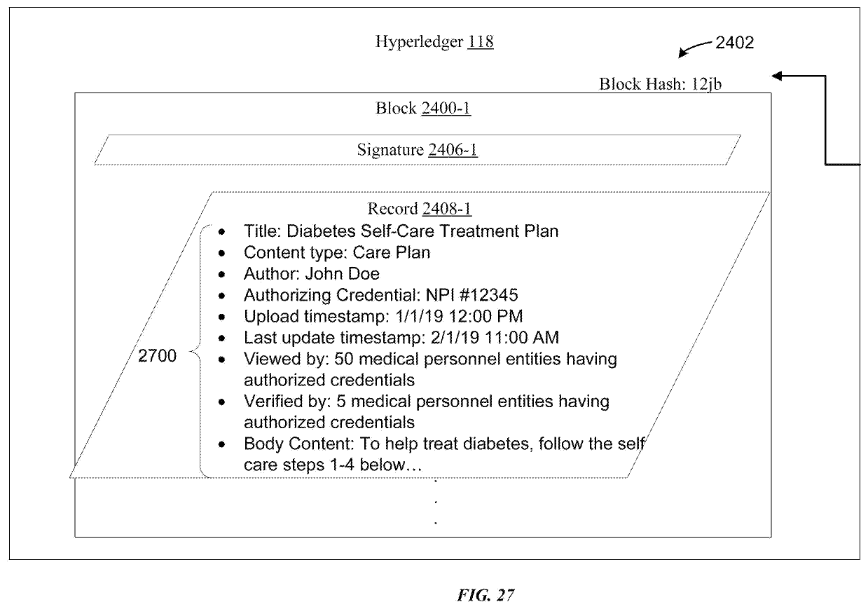

FIG. 27 shows example content 2700 stored on the distributed ledger 118, in accordance with various embodiments. A medical personnel entity may have used a computing device 2310 to submit a transaction request to perform an operation on the distributed ledger 118, where the operation included storing the content 2700 on the distributed ledger 118. One or more rules 2306 may have determined that the requesting medical personnel entity has (i) valid authentication information and/or (ii) valid authorization information, and/or that the content 2700 includes at least a portion of Body Content that is new relative to other content stored in the distributed ledger 118. In the depicted example, the one or more rules 2306 are satisfied, and the content 2700 is stored in the record 2408-1 in the block 2406-1 in the distributed ledger 118. Further, the block 2400-1 includes a signature 2406-1 of the medical personnel entity that provided the content 2700. The signature 2406-1 may be a digital signature that uniquely identifies the medical personnel entity as the source of the content 2700.

In the depicted example, the content 2700 may describe the knowledge graph 2500 that included nodes representing original content related to Type 2 Diabetes. As depicted, the content 2700 may include various information, such as a Title (“Diabetes Self-Care Care plan”), Content Type (“Care plan”), Author (“John Doe”), Authorization information (“NPI #12345”), Upload Timestamp (“1/1/19 12:00 PM”), Last Updated Timestamp (“2/1/19 11:00 AM”), Viewed By (“50 medical personnel entities having authorized credentials”), Verified By (“5 medical personnel entities having authorized credentials”), and/or Body Content (“To help treat diabetes, follow the self-care steps 1-4 below . . . ”).

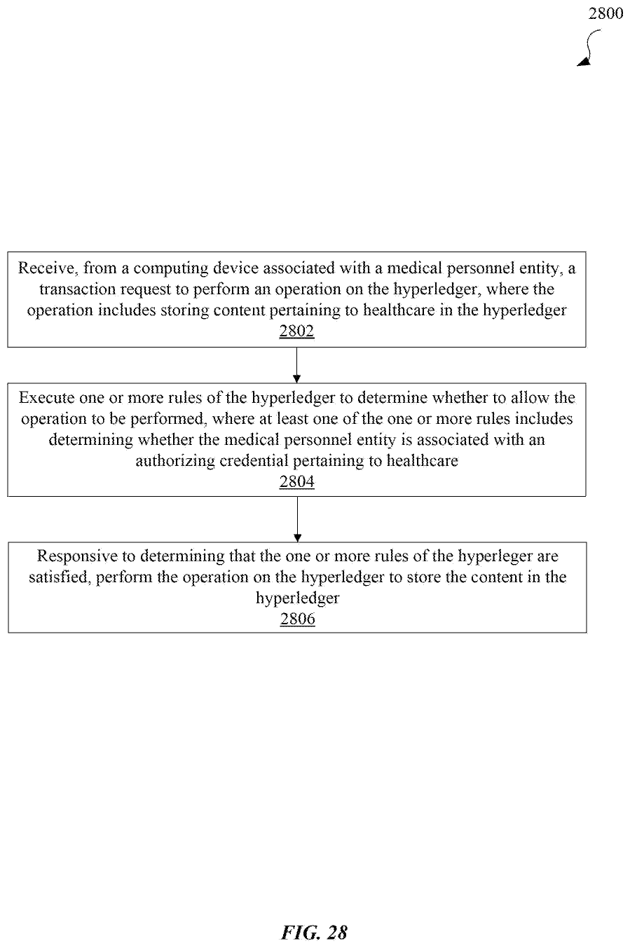

FIG. 28 shows a method 2800 for maintaining content pertaining to healthcare in a distributed ledger 118, in accordance with various embodiments. In some embodiments, the method 2800 is implemented as a computer program product in a non-transitory computer-readable medium and executable by one or more processors of one or more computing devices described in the cognitive intelligence platform 102 of FIG. 1. In some embodiments, the method 2800 of FIG. 28 is implemented as a system for maintaining a distributed ledger 118 for content at one or more nodes 116. The system can include components described in the cognitive intelligence platform 102.

In some embodiments, the method 2800 may involve receiving (2802), from a computing device 2310 associated with a medical personnel entity, a transaction request to perform an operation on the distributed ledger 118, where the operation includes storing content 2700 pertaining to healthcare in the distributed ledger 118. The content 2700 may be any suitable content, such as evidence-based guidelines, knowledge representation, description of a knowledge representation, clinical studies, clinical trial results, clinical process descriptions, care plans for medical conditions, and/or the like. In some embodiments, the content 2700 includes a care plan where at least a portion of the care plan is written by the medical personnel entity. The distributed ledger 118 may maintain other content (e.g., care plans) that are validated as being provided by other licensed medical doctors based on other authorization information that is stored in the distributed ledger 118.

The method 2800 may involve executing (2804) one or more rules 2306 of the distributed ledger 118 to determine whether to allow the operation to be performed, where at least one of the one or more rules includes determining whether the medical personnel entity is associated with authorization information (e.g., medical degree, NPI, medical license number) pertaining to healthcare. Further, the one or more rules may include validating the authorization information with a professional association or government agency that issued the authorization information. The one or more rules 2306 may search the distributed ledger 118 for the authorization information for the medical personnel entity. The authorization information may be provided by the medical personnel entity in the transaction request and included in the content, and/or the authorization information may be stored as part of a record of the distributed ledger 118. This particular rule 2306 may be satisfied when the authorization information of the medical personnel entity is valid. This particular rule 2306 may not be satisfied when the authorization information of the medical personnel entity is not valid. When the authorization information is not valid, the rule 2306 may prevent the content 2700 from being stored on the distributed ledger 118.

The one or more rules 2306 may include determining, using one or more transactions stored in the distributed ledger 118, whether at least a portion of the content 2700 is new relative to other content in the distributed ledger 118. When the content 2700 includes text strings, the content 2700 may be parsed. The text strings may be tokenized into words, keywords, phrases, symbols and other elements. Other preprocessing may be performed such as removing certain words or characters. The strings and/or tokens may be compared to other strings and/or tokens in other content stored in the distributed ledger 118. In some embodiments, the strings and/or tokens may be compared to other content that pertains to the same content type or has a similar title, for example.

When the content 2700 includes a video, the audio of the video may be translated into text and the text may be processed as described above. Object character recognition may be used to identify objects in the video such that the objects may be compared with other videos stored in the distributed ledger 118 to determine if there are matching objects between the videos. When the content 2700 includes images, the images may be compared with other images stored in content in the distributed ledger 118 to determine if the images match.

If the content 2700 does not include at least a portion of new content relative to other content in the distributed ledger 118, the rules 2306 may prevent the content 2700 from being stored on the distributed ledger 118. If at least a portion of the content 2700 is new, then this particular rule may be satisfied. Using this particular rule may ensure that the knowledge and/or content that is added to the distributed ledger 118 is not duplicated and may provide medical personnel entities the ability to own unique content in the distributed ledger 118 to which they may control access.

In some embodiments, prior to allowing the content to be stored in the distributed ledger 118, the one or more rules may include validating authentication information (e.g., username, password, unique identifier) that are provided to the computing device 2310 during registration. The authentication information may be included in the transaction request to perform the operation.

Responsive to determining that the one or more rules 2306 of the distributed ledger 118 are satisfied, the method 2800 may involve performing the operation on the distributed ledger 118 to store the content 2700 in the distributed ledger 118. Responsive to determining that the one or more rules 2306 are not satisfied, the method 2800 may include preventing the operation from being performed on the distributed ledger.

In some embodiments, the method 2800 may include receiving, from the computing device 2310, a second transaction request to perform a second operation on the distributed ledger 118, where the second transaction request includes search criteria, and the second operation includes providing, based on the search criteria, a care plan pertaining to healthcare that is stored in the distributed ledger 118. The method 2800 may also include executing the one or more rules of the distributed ledger 118 to determine whether to allow the operation to be performed, where at least a second rule of the one or more rules includes determining whether the medical personnel entity has a right to access the content. Responsive to determining that the one or more rules 2306 of the distributed ledger 118 are satisfied, the method 2800 may also include performing the second operation on the distributed ledger to provide, based on the search criteria, the care plan pertaining to healthcare to the computing device 2310.

In some embodiments, the method 2800 may include receiving, from the computing device 2310, a second transaction request to perform a second operation on the distributed ledger 118, where the second operation includes storing updated content pertaining to healthcare in the distributed ledger 118, and the updated document adds additional content to original content included in the content 2700 stored in the distributed ledger 118. The method 2800 may also include executing the one or more rules 2306 of the distributed ledger 118 to determine whether to allow the operation to be performed, where at least a second rule of the one or more rules 2306 includes determining whether the additional content in the updated content is new relative to other content pertaining to healthcare stored in the distributed ledger 118. Responsive to determining that the one or more rules 2306 of the distributed ledger 118 are satisfied, the method 2800 may also include performing the second operation on the distributed ledger 118 to store, in the distributed ledger 118, the updated content including the additional content and the original content.

In some embodiments, a timestamp may be stored for the content 2700 in the distributed ledger 118. Any time the content 2700 is updated and/or verified, a new timestamp may be stored with the content 2700, along with identifying information of the medical personnel entity that updated and/or verified the content 2700. This may enable a time series to be maintained for the content 2700 to allow a user to view the history of updates and/or verifications since the content 2700 was stored on the distributed ledger 118.

In some embodiments, the method 2800 may include receiving, from a computing device associated with a second medical personnel entity, a second transaction request to perform a second operation on the distributed ledger 118, where the second operation includes verifying the content 2700 in the distributed ledger 118. The method 2800 may include determining whether the second medical personnel entity is associated with valid authorization information (e.g., medical degree, medical license number, etc.) that is provided in the second transaction request or that is stored in the distributed ledger 118. Responsive to determining that the second medical personnel entity is associated with the valid authorization information, the method 2800 may include performing the second operation on the distributed ledger 118 by allowing the second medical personnel entity to verify the content 2700. A timestamp may be stored with the content 2700 when the content 2700 is verified.

In some embodiments, the method 2800 may include receiving, from a second computing device associated with a user, a transaction request to perform a second operation on the distributed ledger 118, where the second operation includes determining whether the content 2700 is trustworthy. The method 2800 may include determining, using the distributed ledger 118, a source of the content by identifying the medical personnel entity that authored or created the content 2700. The method 2800 may also include determining that the source is associated with valid authorization information (e.g., medical degrees, medical license numbers, etc.). The method 2800 may also include determining whether the content 2700 has been verified within a certain time period by a medical personnel entity with valid authorization information. The method 2800 may also include providing a notification to the second computing device that the content is trustworthy based on the source of the content being associated with the authorization information and the content 2700 being verified within the certain time period.

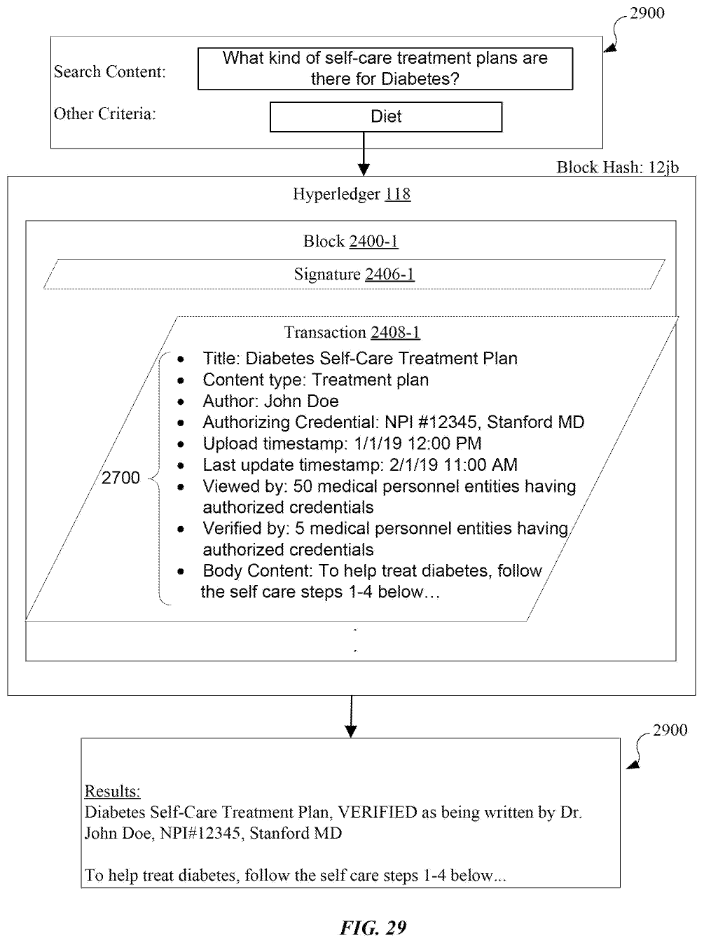

FIG. 29 shows an example search for content 2700, in accordance with various embodiments. As depicted, the content 2700 is stored in the record 2408-1 in the block 2400-1, which includes the signature 2406-1 of the medical personnel entity (e.g., John Doe) that authored and requested the content 2700 be stored in the distributed ledger 118. A medical personnel entity may use a computing device to execute the software application that is logged into the cognitive intelligence platform 102 using the authentication information. The software application may present a search user interface 2900 that includes various input elements, such as text bars that enable natural language text to be entered (e.g., questions, titles, authors, etc.) and/or other search criteria. The other search criteria may enable the user to select a particular diet, exercise, and/or medication that the medical personnel entity may choose for a particular patient having a particular medical condition. The search user interface 2900 may enable a user to search for content of interest that is stored on the distributed ledger 118, to verify the source of searched for content, to verify that the source is associated with valid authorization information, and/or to verify the content has been verified within a certain time period by a medical personnel entity having valid authorization information, among other things.

In the depicted example, the user entered the question “What kind of self-care care plans are there for Diabetes?” and entered “Diet” as another search criteria. The computing device of the user may transmit a transaction request to the distributed ledger 118 via the cognitive agent 110. The critical thinking engine 108, the natural language database 122, and/or the knowledge cloud 106 may be used to identify and understand the question asked in natural language. The distributed ledger 118 may be searched, based on the question, to identify the content 2700 stored in record 2408-1. One or more rules 2306 may be executed to determine whether the requesting user has an access right to the content 2700. If the requesting user has the access right, then the content 2700 may be provided to the computing device of the requesting user and displayed on the search user interface 2900. If the requesting user does not have the access right, then a notification may be transmitted to the computing device of the requesting user prompting the user to purchase the access right.

When the content 2700 is transmitted to the computing device of the requesting user, the search user interface 2900 may present the content in a results section. For example, the results section may present the title “Diabetes Self-Care Care plan”, as well as an indication that the content 2700 is verified as being written by Dr. John Doe, NPI #12345, Stanford MD. The results section may also present the body content “To help treat diabetes, follow the self-care steps 1-4 below . . . ”

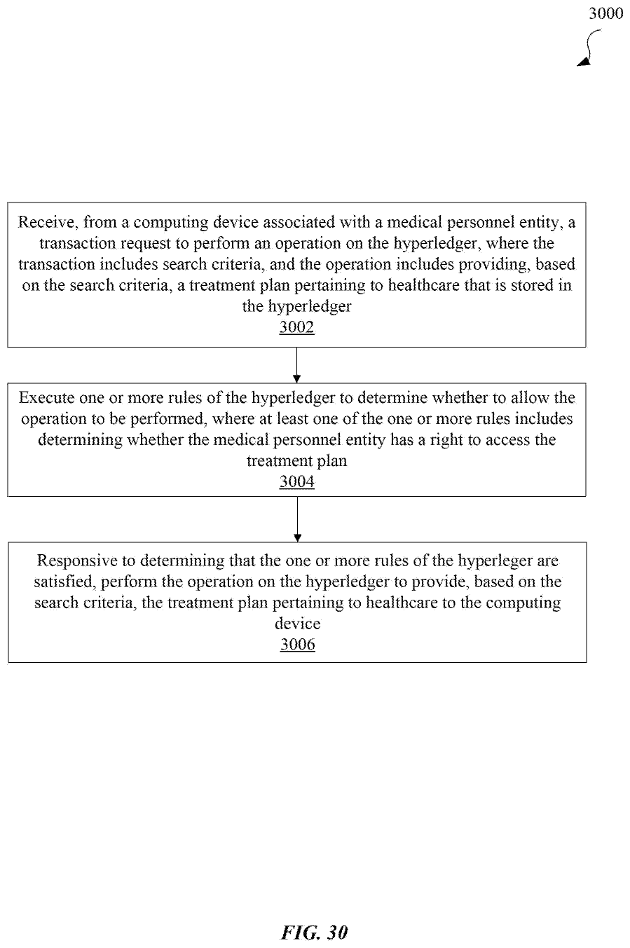

FIG. 30 shows a method 3000 for maintaining content pertaining to healthcare in a distributed ledger 118, in accordance with various embodiments. In some embodiments, the method 3000 is implemented as a computer program product in a non-transitory computer-readable medium and executable by one or more processors of one or more computing devices described in the cognitive intelligence platform 102 of FIG. 1. In some embodiments, the method 3000 of FIG. 30 is implemented as a system for maintaining a distributed ledger 118 storing content pertaining to healthcare at one or more nodes 116. The system can include components described in the cognitive intelligence platform 102.

In some embodiments, the method 3000 may involve receiving (3002), from a computing device 2310 associated with a medical personnel entity, a transaction request to perform an operation on the distributed ledger 118, where the transaction includes search criteria, and the operation includes providing, based on the search criteria, content pertaining to healthcare that is stored in the distributed ledger 118. In other embodiments, the search request may be for any suitable content (e.g., evidence-based guidelines, clinical studies, clinical trials, clinical techniques, knowledge representations (graphs), care plans, etc.) pertaining to healthcare.

The method 3000 may involve executing (3004) one or more rules of the distributed ledger 118 to determine whether to allow the operation to be performed, where at least one of the one or more rules includes determining whether the medical personnel entity has a right to access the content. The requesting medical personnel entity may have a right to access the content 2700 when they purchase the right, such that the creator of the unique knowledge in the content 2700 may be rewarded for sharing the knowledge. In other embodiments, the requesting medical personnel entity may have a right to access the content 2700 when the requesting medical personnel entity is associated with an organization (e.g., a hospital) that has viewing privileges of the content 2700, the requesting medical personnel entity is associated with authorization information pertaining to healthcare, the requesting medical personnel entity is associated with a same organization (e.g., hospital) as the creator of the content 2700, or some combination thereof.

Responsive to determining that the one or more rules of the distributed ledger 118 are satisfied, the method 3000 may involve performing the operation on the distributed ledger 118 to provide, based on the search criteria, the content 2700 pertaining to healthcare to the computing device 2310. Responsive to determining that the one or more rules of the distributed ledger 118 are not satisfied, the method 3000 may not perform the operation.

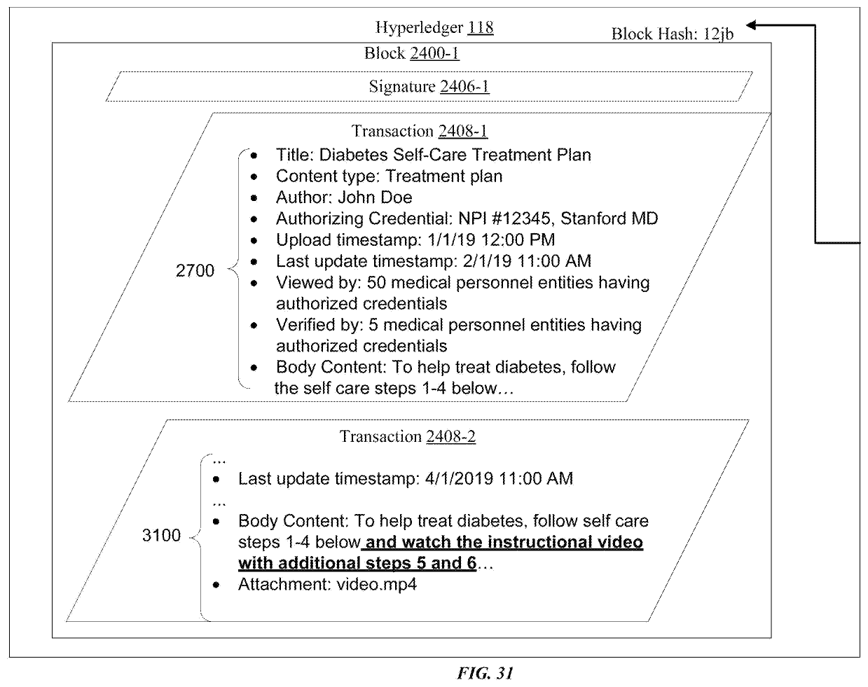

FIG. 31 shows an example updated content 3100 stored in the distributed ledger 118, in accordance with various embodiments. A medical personnel entity may have used a computing device 2310 to submit a transaction request to perform an operation on the distributed ledger 118, where the operation included storing the content 2700 on the distributed ledger 118. One or more rules 2306 may have determined that the requesting medical personnel entity has (i) valid authentication information and/or (ii) valid authorization information, and/or that the content 2700 includes at least a portion of Body Content that is new relative to other content stored in the distributed ledger 118. In the depicted example, the one or more rules 2306 are satisfied, and the content 2700 is stored in the record 2408-1 in the block 2406-1 in the distributed ledger 118. Further, the block 2400-1 includes a signature 2406-1 of the medical personnel entity that provided the content 2700. The signature 2406-1 may be a digital signature that uniquely identifies the medical personnel entity as the source of the content 2700.

In the depicted example, the medical personnel entity may have used the computing device 2310 to submit a second transaction request to perform a second operation on the distributed ledger 118, where the second operation included storing updated content 3100 on the distributed ledger 118. Updated content 3100 may include original content (“To help treat diabetes, follow self-care steps 1-4 below”), as well as additional content description and video. The additional description says “and watch the instructional video with additional steps 5 and 6” (which has been bolded and underlined).

One or more rules 2306 may have determined that the requesting medical personnel entity has (i) valid authentication information and/or (ii) valid authorization information, and/or that the content 3100 includes at least a portion of Body Content that is new relative to other content stored in the distributed ledger 118. The additional description and video may be new relative to other content in the distributed ledger 118. In the depicted example, the one or more rules 2306 are satisfied, and the content 3100 is stored in the record 2408-2 in the block 2406-1 in the distributed ledger 118. Further, the block 2400-1 includes the signature 2406-1 of the medical personnel entity that provided the content 2700 and 3100. The signature 2406-1 may be a digital signature that uniquely identifies the medical personnel entity as the source of the content 2700 and 3100. In some embodiments, other medical personnel entities may provide the other content, which may be added to the block 2400-1 or other blocks 2400 if the one or more rules 2306 are satisfied, and a signature 2406 for those other medical personnel entities may be stored with their respective content.

In the depicted example, the updated content 3100 may be related to the additional content (represented by dotted line 2552) that included nodes representing additional content related to Type 2 Diabetes in the knowledge graph 2550 of FIG. 25B. Further, content 3100 may include a video 2612 that presents instructions for the new self-care steps. The content 3100 may include various information, some of which is the same as the content 2700. Since the content 3100 is provided from the same medical personnel entity, John Doe, the author and authorization information may be the same. The title and content type may remain the same, since the additional content adds on to the original content for the new self-care treatment of Type 2 Diabetes. As depicted, the last update timestamp may be updated to a current timestamp for the update (“4/1/2019 11:00 AM”). Further, the Body Content section includes updated text, as noted above, and an Attachment includes the video file (“video.mp4”) that may be embedded in a document including the description of the care plan in the updated content 3100.

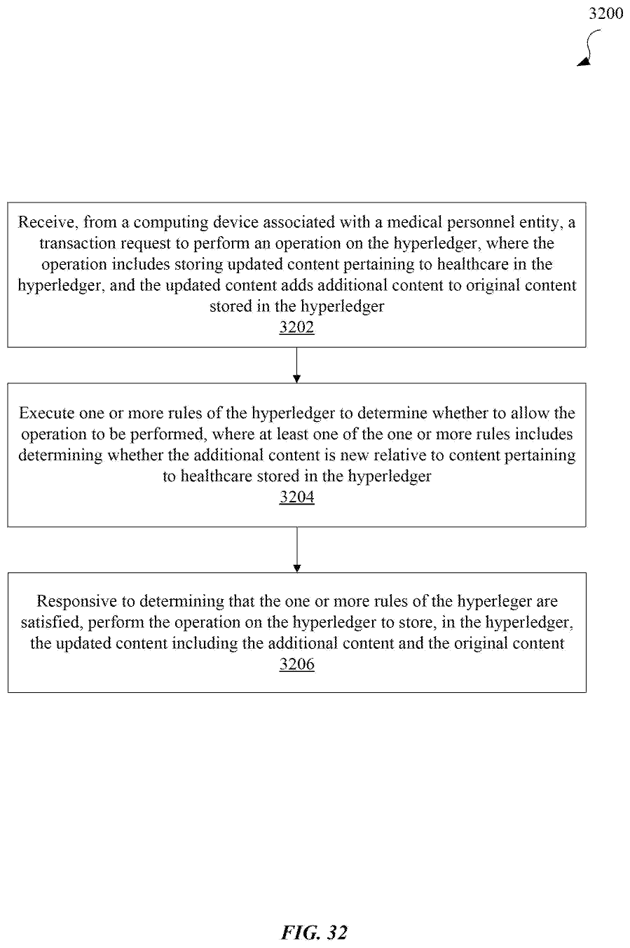

FIG. 32 shows a method 3200 for maintaining content pertaining to healthcare in a distributed ledger 118, in accordance with various embodiments. In some embodiments, the method 3200 is implemented as a computer program product in a non-transitory computer-readable medium and executable by one or more processors of one or more computing devices described in the cognitive intelligence platform 102 of FIG. 1. In some embodiments, the method 3200 of FIG. 32 is implemented as a system for maintaining a distributed ledger 118 storing content pertaining to healthcare at one or more nodes 116. The system can include components described in the cognitive intelligence platform 102.

In some embodiments, the method 3200 may involve receiving (3202), from a computing device 2310 associated with a medical personnel entity, a transaction request to perform an operation on the distributed ledger 118, where the operation includes storing updated content 3100 pertaining to healthcare in the distributed ledger 118, and the updated content 3100 adds additional content to original content stored in the distributed ledger 118. The additional content may be any suitable text, video, or images describing or illustrating knowledge pertaining to healthcare (e.g., evidence-based guidelines, clinical processes, clinical trials, knowledge representations, etc.).

The method 3200 may involve executing (3204) one or more rules 2306 of the distributed ledger 118 to determine whether to allow the operation to be performed, where at least one of the one or more rules includes determining whether the additional content is new relative to content pertaining to healthcare stored in the distributed ledger 118. The one or more rules may identify the additional content in the updated content 3100 and preprocess the additional content as described above for videos, text strings, and/or images to determine whether the additional content is new relative to the other content in the distributed ledger 118.

Responsive to determining that the one or more rules 2306 of the distributed ledger 118 are satisfied, the method 3200 may involve performing the operation on the distributed ledger 118 to store, in the distributed ledger 118, the updated content 3100 including the additional content and the original content. A timestamp of when the updated content 3100 is stored in the distributed ledger 118 may be stored with the updated content 3100, such that a time series of the evolution of the content may be maintained. The identity and the authorization information of the medical personnel entity that provided the updated content 3100 may also be stored with the content 3100. Responsive to determining that the one or more rules 2306 of the distributed ledger 118 are not satisfied, the operation to store the updated content 3100 may not be performed.

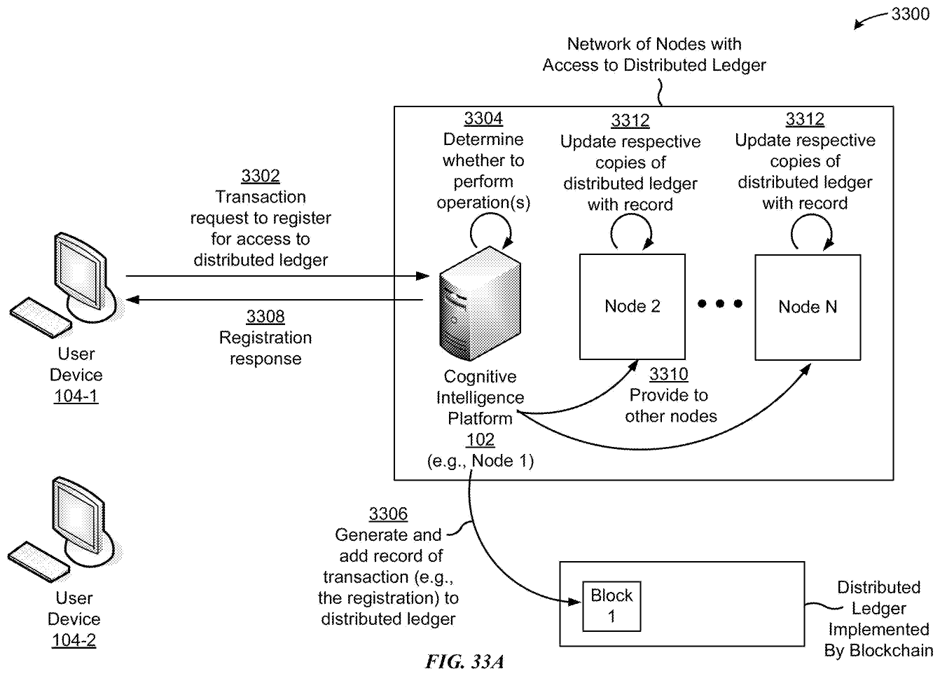

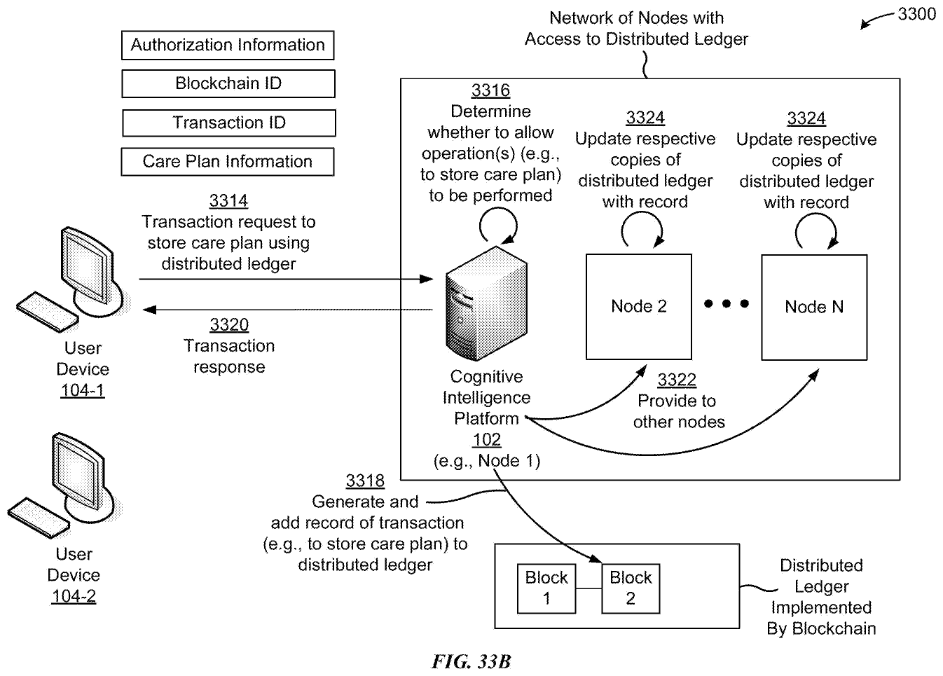

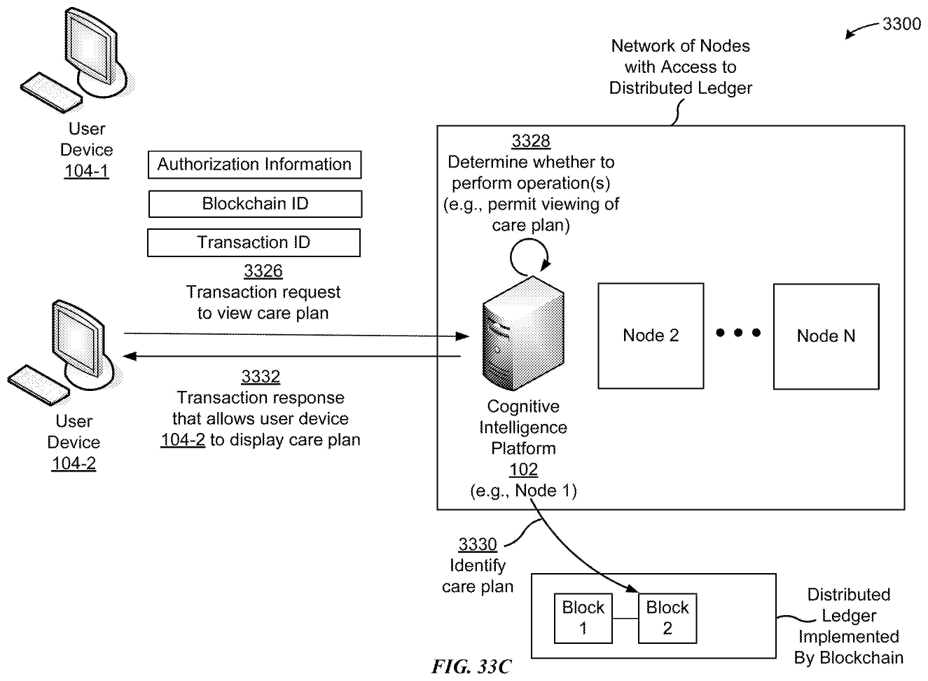

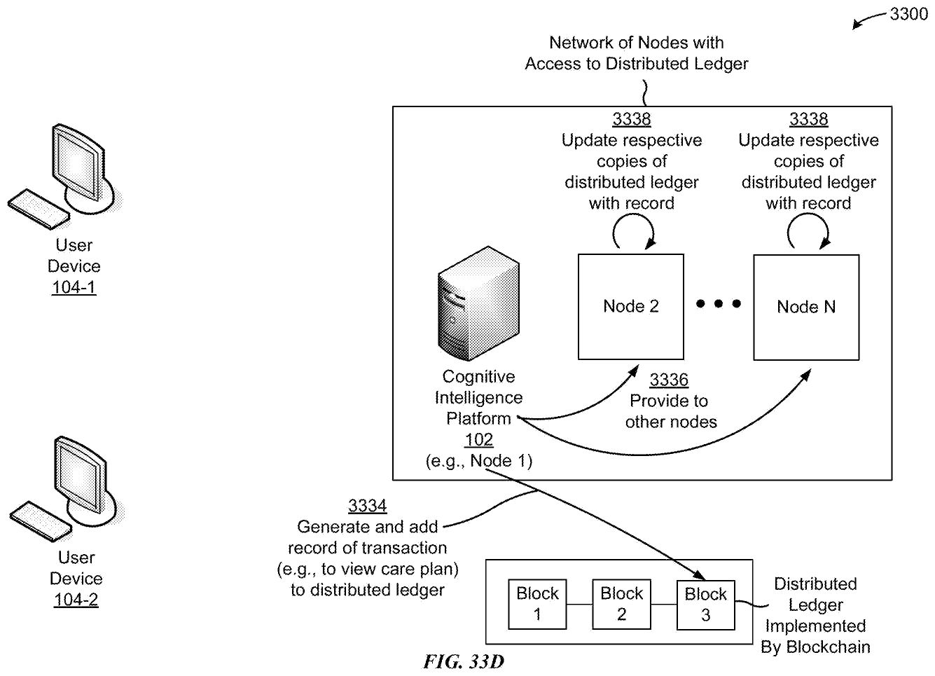

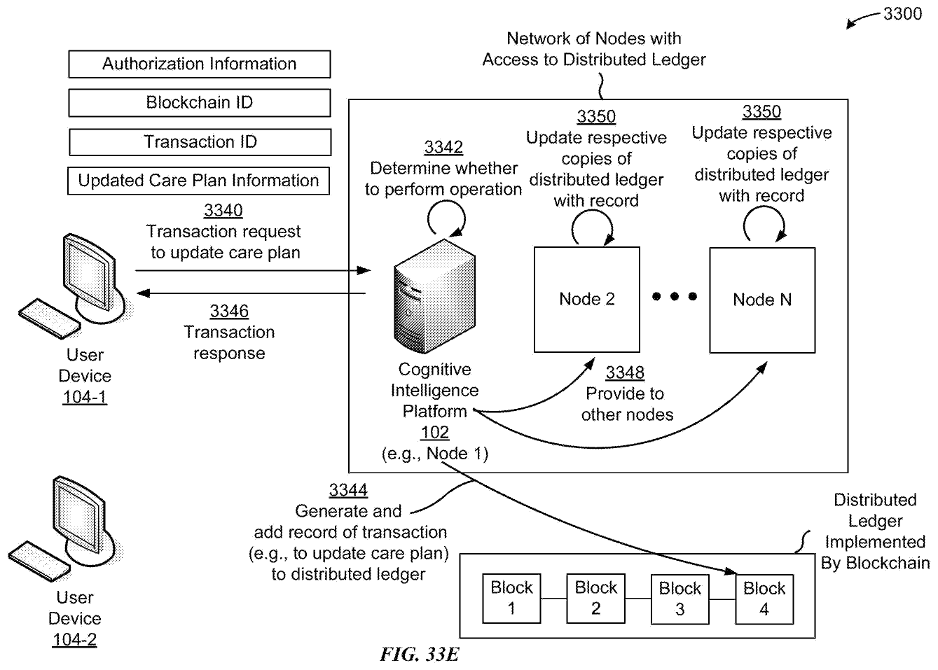

FIGS. 33A-33E are diagrams of one or more example embodiments 3300 described herein. The example embodiment(s) 3300 may include a network of nodes that have access to a distributed ledger (shown as Node 1 through Node N, where Node 1 is the cognitive intelligence platform 102), a first user device operated by a medical professional (referred to hereafter as user device 104-1), and a second user device (referred to hereafter as user device 104-2) operated by another user, such as a client of the medical professional, another medical professional, and/or the like.

In some embodiments, the network may be a distributed, decentralized network. In some embodiments, the cognitive intelligence platform 102 may serve as a parent node or management node for the network. In some embodiments, each node in the network may maintain a respective copy of the distributed ledger. In some embodiments, the user device 104-1 and/or the user device 104-2 may be nodes in the network. In other embodiments, the user device 104-1 and/or the user device 104-2 may be devices outside of the network and may communicate with one or more of the nodes.

In some embodiments, the distributed ledger may be implemented by a tamper-resistant data structure, such as a blockchain, to provide a secure way to store, update, view, and/or verify content. The blockchain may include a permissioned blockchain, a federated blockchain, a distributed blockchain, a private blockchain, a hybrid blockchain, and/or the like. For example, the blockchain may be a permissioned blockchain (e.g., a hyperledger) such that only authorized users have permission to engage in particular transactions. The transactions may include using the distributed ledger to store the content, update the content, view the content, verify the content and/or updated content, and/or the like, as will be described further herein. The blockchain may include a continuously-growing list of records, called blocks, that may be linked together to form a chain. In some cases, each block in the blockchain may include a timestamp and a link to a previous block. The blocks may be secured from tampering and revision. For example, the blocks in the blockchain may be encrypted using public keys, such that accessing a block would require using a private key to decrypt the block. Additional information regarding securely storing the content is provided in FIG. 24.

Some embodiments described herein may involve communications between devices, such as a communication between two nodes in the network, a communication between a device outside of the network and a node, and/or the like. These communications may be performed via a communication interface, such as an application programming interface (API), a radio interface (e.g., that supports individual message transmissions, message broadcasts, etc.), and/or another type of communication interface.

In some embodiments, the content stored by the distributed ledger may include care plans of medical professionals, evidence-based guidelines, knowledge representation (e.g., using knowledge graphs), patient information (e.g., using patient graphs), clinical information (e.g., for clinical studies, clinical processes, clinical techniques, etc.), and/or the like. In one or more embodiments shown in FIGS. 33A-33E, the content may include one or more versions of a care plan of the medical professional.

In some embodiments, the care plans that are included in the content may be partially or wholly electronically generated by the cognitive intelligence platform 102. For example, the cognitive intelligence platform 102 may receive data pertaining to a patient that is entered by a medical professional, and may analyze the data in conjunction with a knowledge graph for a medical condition associated with the data and/or a patient graph associated with the patient and the medical condition. The cognitive intelligence platform 102 may electronically generate a care plan including action instructions (e.g., take a certain medication, perform certain labs, perform certain self-help, etc.) for the patient based on the comparison of the patient graph and the knowledge graph for the medical condition. Also, the data entered by the medical professional may be translated into various medical codes in real-time or near real-time and the various codes may be used when electronically generated the care plan. For example, if a particular medical code indicates the patient has already had a certain test performed for a medical condition, the cognitive intelligence platform 102 may identify another type of test having a different medical code in a knowledge graph of the medical condition to recommend performing for the patient.

In some embodiments, a medical professional may review the electronically generated care plan and make modifications to the care plan. The modified care plan may include certain action instructions that were electronically generated by the cognitive intelligence platform 102 and certain action instructions that were generated and/or modified by the medical professional. The medical professional may make the modifications based on their knowledge, experience, and/or personal preferences for care regimens, medical practices, etc. As result, the resulting care plans may constitute intellectual property for the medical professionals, and as such, the medical professional may benefit using the disclosed techniques to securely store and/or provide access to the care plans via the blockchain.

FIG. 33A illustrates a registration procedure that allows the medical professional to register for access to the distributed ledger. In some embodiments, and as shown by reference number 3302, the medical professional may interact with an interface of user device 104-1 to register for access to the distributed ledger. For example, the medical professional may input certain credential information that may be used to authorize and/or authenticate transactions.

The credential information may include authentication information, authorization information, and/or the like. The authentication information may include information used to verify an identify of a user, such as a driver's license number, a social security number, a name, an insurance provider number, an address, a medical record number, and/or the like. The authorization information may include information used to verify a qualification of a user, such a National Provider Identifier (NPI), a license number, a date licensed, a date the license was last updated, a medical practice specialization, a location of practice, and/or the like.

In some embodiments, the type of user may dictate a type of access that the user has to the distributed ledger. For example, a medical professional may have read access to view and/or verify care plans, while a patient may only have read access to view care plans.

Some embodiments described herein may refer to receiving, processing, generating, and/or providing personal information of individuals. It should be understood that any use of the personal information may be subject to consent of the individuals and will be used in a manner that is compliant with applicable laws concerning protection and/or use of personal information. As an example, consent of an individual may be obtained as part of an “opt-in” clause, as a prompt of a user registration screen, and/or the like. Furthermore, any use and/or transmission of the personal information may be secured via one or more encryption techniques or techniques to anonymize the personal information.

In some embodiments, a node in the network may utilize a computer-implemented application for performing operations on the distributed ledger. For example, the medical professional may use the application to submit a transaction request to perform an operation on the distributed ledger. The application may be written in computer instructions that are stored on one or more memory devices of the node and executable by one or more processing devices of the node. In some embodiments, the application may be a stand-alone application that is installed on the node, while in other embodiments, the application may be executable via another application (e.g., a website in a web browser). In some embodiments, the application may include instructions for determining whether to provide a user with access to the distributed ledger (e.g., based on whether the user provided valid and/or verifiable credential information).

In some embodiments, and as shown by reference number 3304, the cognitive intelligence platform 102 may determine whether to allow one or more operations to be performed based on the transaction request to provide the medical professional with access to the distributed ledger. For example, the cognitive intelligence platform 102 may, based on information included in the registration request, determine whether to allow the one or more operations to be performed to grant the medical professional with access to the distributed ledger. To make the determination, the cognitive intelligence platform 102 may verify the credential information of the medical professional. For example, the cognitive intelligence platform 102 may compare authorization information of the medical professional to a publicly accessible data source that stores corresponding authorization information of registered medical professionals. The authorization information of the medical professional may be verified if, for example, the received authorization information matches the corresponding stored authorization information.

In some embodiments, and as shown by reference number 3306, the cognitive intelligence platform 102 may generate and add a record of the transaction to the distributed ledger. For example, the cognitive intelligence platform 102 may generate a record indicating that the medical professional has been granted access to the distributed ledger and may add the record to the distributed ledger. The cognitive intelligence platform 102 may add the record by storing the record as a block in a blockchain associated with the medical professional.

The record may include the credential information of the medical professional, a blockchain identifier for the blockchain associated with the medical professional, the transaction identifier associated with the transaction request, time information (e.g., a time stamp indicating when the transaction request was made, a time stamp indicating when the transaction request was approved, etc.), and/or the like.

In some embodiments, the cognitive intelligence platform 102 may generate a smart contract. For example, the cognitive intelligence platform 102 may generate a smart contract based on a set of pre-defined guidelines made by a board of medical professionals that oversee the distributed ledger, based on user preferences of the medical professional (e.g., the medical professional might specify a cost that other users must pay to view the content), and/or the like.

A smart contract may refer to a computer protocol with one or more functions capable of digitally facilitating, verifying, and/or assisting with transactions associated with the distributed ledger. The smart contract may include a function configured to authorize and/or authenticate a transaction request made by a user, a function configured to add content to the distributed ledger (e.g., a care plan, an updated care plan that includes a modification relative to the new care plan, and/or the like), a function configured to verify the content, a function configured to allow certain authorized users to view the content, a function configured to incentivize one or more transactions, and/or the like.

In some embodiments, the cognitive intelligence platform 102 may include the smart contract as part of the record of the transaction and/or as a separate record. While one or more embodiments described herein refer to a smart contract, it is to be understood that this is provided by way of example. In other embodiments, for example, one or more functions described as being part of the smart contract may be implemented as part of critical thinking engine 108 and/or another type of configurable rules engine.

In some embodiments, and as shown by reference number 3308, the cognitive intelligence platform 102 may provide, to the user device 104-1, a registration response indicating whether the medical professional has been registered and/or has been provided with access to the distributed ledger. The user device 104-1 may display the registration response on an interface to notify the medical professional of the result of the registration procedure.

In some embodiments, and as shown by reference number 3310, the cognitive intelligence platform 102 may provide the record that was added to the distributed ledger to one or more other nodes in the network (shown as Node 2 through Node N). For example, the cognitive intelligence platform 102 may provide the record to one or more other nodes to permit the one or more other nodes to update independent copies of the distributed ledger to include the record.

In some embodiments, providing the record may include separate data transmissions to each respective node. In other embodiments, providing the record may include broadcasting the node such that each respective node is able to tune in to the broadcast and receive the record. In some embodiments, the entire distributed ledger that includes the record may be provided to the one or more other nodes.

In some embodiments, a group of nodes in the network may validate and/or verify the record of the transaction that provided the medical professional with access to the distributed ledger. For example, the group of nodes in the network may use a consensus protocol to independently validate and/or verify the record. The consensus protocol may be used to create a record of consensus with a cryptographic audit trail that is maintained and validated by the group of nodes. This means that if a user adds a new block to the blockchain, nodes associated with each authorized user may have to independently update a separate copy of the blockchain and may have to independently verify that the separate copy of the blockchain matches the blockchain that has been modified to include the new block.

In some embodiments, the group of nodes may use the consensus protocol to independently validate and/or verify the transaction after the cognitive intelligence platform 102 has performed the one or more operations. In other embodiments, the group of nodes may use the consensus protocol to independently validate and/or verify the transaction request (e.g., before the cognitive intelligence platform 102 is permitted to perform the one or more operations).

In some embodiments, and as shown by reference number 3312, the one or more other nodes may update respective copies of the distributed ledger with the record. For example, the one or more other nodes may update respective copies of the distributed ledger such that each node has an independently verifiable copy of the distributed ledger that includes the record. By updating respective copies of the distributed ledger, each node may independently verify the record that has been added to the distributed ledger, thereby improving security and providing failover protection (e.g., if the cognitive intelligence platform 102 fails, another node may become the parent or management node and may perform one or more actions described herein as being performed by the cognitive intelligence platform 102).

In this way, the cognitive intelligence platform 102 provides the medical professional with access to the distributed ledger and adds a verifiable record of the medical professional's registration to the distributed ledger. Once registration is completed, permitted users (e.g., the medical professional, clients of the medical professional, and/or the like) will be able to engage in various transactions, such as using the distributed ledger to store the care plan, to update the care plan, to view the care plan, and/or the like, as will be shown further herein.

FIG. 33B illustrates a transaction that involves storing content (e.g., a care plan) of the medical professional as part of the distributed ledger. In some embodiments, and as shown by reference number 3314, the cognitive intelligence platform 102 may receive, from user device 104-1, a transaction request to store the care plan using the distributed ledger. For example, the medical professional may create a transaction request by inputting credential information to an interface displayed on user device 104-1. When the medical professional submits the transaction request, the user device 104-1 may provide the transaction request to the cognitive intelligence platform 102.

In some embodiments, the transaction request may include the credential information of the medical professional (e.g., shown as authorization information), the blockchain identifier associated with the blockchain used to store records associated with the care plan, a transaction identifier that indicates a type of transaction being requested, care plan information, and/or the like. The care plan information may indicate a title, an author, actual content included within the care plan, and/or the like. As discussed above, in some embodiments, a portion of the actual content included within the care plan may be electronically generated by the cognitive intelligence platform 102 and/or a portion of the actual content included within the care plan may be generated by the medical professional.

In some embodiments, and as shown by reference number 3316, the cognitive intelligence platform 102 may determine whether to allow one or more operations to be performed based on the transaction request to store the care plan. For example, the cognitive intelligence platform 102 may process the transaction request to identify a type of transaction being requested. The transaction request may, for example, include an identifier associated with a request to store new content, such as the care plan.

In some embodiments, if at least a portion of the care plan is new content for a medical condition relative to other care plans for the medical condition stored in the blockchain, then the care plan may be added to the blockchain. In some embodiments, just the portion of the care plan that is new for the medical condition relative to the other care plans for the medical condition may be stored in the blockchain. In some embodiments, just the portion of the care plan that is new (e.g., not already part of a care plan stored on the blockchain) and that is determined to be added by a medical professional may be stored in the blockchain. In such an instance, the authorization information of the medical professional, an identity of the medical professional, and/or any other suitable information of the medical professional that generated the portion of the care plan may be stored with the portion on the blockchain.

In some embodiments, the cognitive intelligence platform 102 may determine whether to grant the transaction request to store the care plan based on whether the credential information provided in the transaction request matches corresponding credential information stored using the distributed ledger. The corresponding credential information may be stored in the record that was generated when the medical professional registered for access to the distributed network and/or may be stored in another data structure accessible to the cognitive intelligence platform 102.

In some embodiments, the cognitive intelligence platform 102 may use the smart contract to determine whether to grant the transaction request to store the care plan. For example, the cognitive intelligence platform may provide information included in the transaction request as input to one or more functions of the smart contract. The smart contract may process the information and may output a value indicating whether to grant the transaction request. As an example, the received authorization information might be provided as input to a verification function of the smart contact, which may cause the smart contract to compare the received authorization information for the medical professional with corresponding stored authorization information. The smart contract may output a value indicating whether to grant the transaction request based on whether the received authorization information and the corresponding stored authorization information matches or satisfies a threshold level of similarity with each other.

In some embodiments, and as shown by reference number 3318, the cognitive intelligence platform may generate and add a record of the transaction to the distributed ledger. For example, the cognitive intelligence platform 102 may add, to the distributed ledger, a record that includes transaction type information indicating the type of transaction request that was made, time information indicating a time during which the transaction request was made and/or granted, the care plan information, and/or the like. The cognitive intelligence platform 102 may add the record to the distributed ledger by storing the record as a block in the blockchain (e.g., as a second block in the blockchain). The second block may be cryptographically linked to the first block in a manner described elsewhere herein.

The record may include the credential information of the medical professional, the blockchain identifier for a blockchain associated with the medical professional, the transaction identifier associated with the transaction request, the care plan information, a verification indicator that indicates whether the care plan has been verified by one or more other licensed professionals, identifiers and/or credential information for the licensed professionals who provided verification, one or more incremental counter values, and/or the like. The one or more incremental counter values may include a value indicating a number of views of the care plan, a value indicating a number of views by individuals with valid authorized credentials, a value indicating a number of verifications made by the individuals with valid authorized credentials, and/or the like.

In the example shown, the blockchain for the medical professional may have a first block associated with the medical professional's registration and may have a second block associated with the transaction to store the care plan. The first and second block may be cryptographically linked in a manner described elsewhere herein (see, e.g., FIG. 24).

In some embodiments, and as shown by reference number 3320, the cognitive intelligence platform 102 may provide the user device 104-1 with a transaction response. The transaction response may, for example, indicate whether the care plan has been stored using the distributed ledger.

In some embodiments, and as shown by reference number 3322, the cognitive intelligence platform 102 may provide, to the one or more other nodes, the record of the transaction involving storing the care plan. In some embodiments, and as shown by reference number 3324, the one or more nodes may update respective copies of the distributed ledger with the record. In some embodiments, the one or more nodes (or a select group of nodes in the network) may use the consensus protocol to independently validate and/or verify the record.

In some embodiments, the cognitive intelligence platform 102 may store the care plan using a distributed file system that is linked to the distributed ledger. For example, if the blockchain is unable to support large quantities of information (or is unable to efficiently handle queries based on the large quantities of information), a distributed file system may be used to store any records associated with the medical professional. The distributed file system may store records using one or more data structures, such as a tree (e.g., a binary search tree (BST), a red-black (RB) tree, a B-tree, and/or the like), a graph, a distributed database, a hash table, a linked list, and/or the like.

To use the distributed file system to store particular information (e.g., care plan information, credential information, and/or the like), the cognitive intelligence platform 102 may use a content addressing technique (or a similar type of hash technique) to process information included in the transaction request, which may generate a cryptographic hash value (e.g., a hashed address) that serves as a pointer to a memory location at which the particular information is to be stored in the distributed file system. The cryptographic hash value may be stored in the blockchain as part of the record indicating that the care plan of the medical professional has been stored. By using the distributed file system for data storage, the network of nodes improves in scalability, conserves processing and/or network resources by being able to perform faster queries, conserves memory resources that might otherwise be used to attempt to store data on the blockchain, and/or the like.

In this way, the cognitive intelligence platform 102 adds a verifiable record of the care plan to the distributed ledger (and/or to the distributed file system) and distributes the verifiable record to the one or more other nodes in the network.

FIG. 33C illustrates a transaction to provide a permitted user with read access needed to view the care plan. The permitted user may be a patient of the medical professional, another medical professional that wants to utilize, learn from, and/or verify the medical professional's care plan, and/or the like. A user may be a permitted user if that user is given an access right to the content. The user may, for example, be given the access right based on being a patient of the medical professional, based on being part of the same organization as the medical professional, based on purchasing the access right (e.g., using currency, digital currency such as cryptocurrency, etc.), and/or the like.

In some embodiments, and as shown by reference number 3326, the cognitive intelligence platform 102 may receive, from user device 104-2, a transaction request to view the care plan. For example, if another user (e.g., a patient, another medical professional, and/or the like) wants to view the care plan, the other user may interact with an interface of user device 104-2 to input credential information as part of a transaction request to view the care plan. When the other user causes a device to submit the transaction request, the transaction request may be provided to the cognitive intelligence platform 102. In some embodiments, such as when the other user has to purchase an access right to view the care plan, the other user may have to provide cryptocurrency (or approval to use the other user's cryptocurrency) in the transaction request.

In some embodiments, the other user may interact with the user device 104-2 to submit a question in natural language asking whether the care plan is a trustworthy care plan. In some embodiments, the other user may interact with the user device 104-2 to request a recommendation as to a particular care plan (e.g., from a set of available care plans stored using the distributed ledger).

In some embodiments, and as shown by reference number 3328, the cognitive intelligence platform 102 may determine whether to allow one or more operations to be performed based on the transaction request to view the care plan. For example, the cognitive intelligence platform 102 may determine whether to permit the other user to view the care plan by processing information included in the transaction request.

In some embodiments, the cognitive intelligence platform 102 may determine whether to permit the other user to view the care plan by using a smart contract to process the information included in the transaction request. For example, the cognitive intelligence platform 102 may provide information included in the transaction request as input to the smart contract to cause the smart contract to output a value indicating whether the other user has permission to view the care plan. The information input to the smart contract may include credential information of the other user, the blockchain identifier associated with the medical professional, another type of identifier of the medical professional, payment information, and/or the like. In some embodiments, the payment information may include cryptocurrency of the other user, approval to deduct cryptocurrency from an account of the other user, an identifier for a virtual wallet used to store the other user's cryptocurrency, and/or the like.

In some embodiments, and as shown by reference number 3330, the cognitive intelligence platform 102 may identify the care plan in the distributed ledger. For example, the cognitive intelligence platform 102 may, using a blockchain identifier associated with the medical professional, reference the blockchain to identify the care plan. The blockchain identifier may identify a set of blocks associated with the medical professional and/or may identify a specific block used to store the care plan. In some embodiments, the blockchain identifier may have been provided in the transaction request. In some embodiments, the request may include another type of identifier associated with the medical professional and the cognitive intelligence platform 102 may reference a data structure that associates the other type of identifier and the blockchain identifier.

In some embodiments, the cognitive intelligence platform 102 may have received information specifying a question from the other user regarding a level of trustworthiness of the care plan. The information may be received at the cognitive agent 110 of the cognitive intelligence platform 102 and may process the information using the critical thinking engine 108, natural language database 122, knowledge cloud 106, and/or conversation orchestrator 124 (e.g., using any of the methods disclosed herein). The cognitive intelligence platform 102 may, for example, determine the level of trustworthiness of the care plan based on verifying that the care plan was generated by a user that has valid credential information and/or by determining that the most recently stored copy of the care plan is updated or current (e.g., by determining if the care plan has been updated/verified within a certain time period).

In some embodiments, the cognitive intelligence platform 102 may have received a transaction request for a recommended care plan. In this case, the cognitive intelligence platform 102 may identify a recommended care plan for the other user by utilizing critical thinking engine 108, natural language database 122, an artificial intelligence engine, and/or the like. For example, a ranking technique may be used to identify the care plan that is associated with a highest number of total views by users (e.g., patients, medical professionals, and/or the like), a care plan with a highest number of total views by medical professional having valid authorization information, a care plan with a highest number of total verifications by medical professionals with valid authorization information, and/or the like. Recommending the care plan that has been viewed by more medical professionals overtime (e.g., relative to other care plans) may expedite a time needed for the other user to obtain trustworthy content. As a result, processing, memory, and/or network resources may be saved by providing more trustworthy content using the distributed ledger because the other user may not have to perform multiple searches to find other care plans if the other user is initially provided with the most trustworthy care plan.

In some embodiments, the cognitive intelligence platform 102 may provide an incentive to medical professionals to view the care plan, to independently verify and/or review the care plan, and/or the like. For example, if a second medical professional views and/or verifies the care plan, the cognitive intelligence platform 102 may cause a virtual wallet associated with the second medical professional to be credited with cryptocurrency in exchange for viewing and/or verifying the care plan. By incentivizing medical professionals to view and/or review content stored on the distributed ledger, the number of views and/or number of verifications linked to the content may serve as a reliable indicator of a level of trust worthiness of the content. This reduces a utilization of resources (e.g., processing resources, network resources, memory resources, and/or the like) that might otherwise be expended were users to have to look at a collection of content (e.g., care plans) in order identify an optimal care plan.

In some embodiments, and as shown by reference number 3332, the cognitive intelligence platform 102 may provide the user device 104-2 with a transaction response that allows the user device 104-2 to display the care plan. In some embodiments, the other user may have an option to download the care plan. In some embodiments, the cognitive intelligence platform 102 may cause an indication of the level of trust worthiness of the care plan to be displayed on the user device 104-2. In some embodiments, the level of trust worthiness of the care plan may be displayed relative to other care plans of other medical professionals. In some embodiments, the cognitive intelligence platform 102 may cause the recommended care plan to be displayed on the user device 104-2.

In this way, the cognitive intelligence platform 102 allows the user device 104-2 to display the care plan in a manner that may be viewed by the permitted user.

FIG. 33D illustrates generating and adding the record of the transaction allowing the permitted user to view the care plan to the distributed ledger and providing the one or more other nodes with the record. In some embodiments, and as shown by reference number 3334, the cognitive intelligence platform 102 may generate and add a record of the transaction to the distributed ledger. For example, the cognitive intelligence platform 102 may generate and add a record of the transaction as a third block in the blockchain associated with the medical professional. The third block may be cryptographically linked to the second block in a manner described elsewhere herein. In some embodiments, the cognitive intelligence platform 102 may store the record using the distributed file system that is linked to the distributed ledger.

In some embodiments, and as shown by reference number 3336, the cognitive intelligence platform 102 may provide, to the one or more other nodes, the record of the transaction allowing the permitted user to view the care plan. In some embodiments, and as shown by reference number 3338, the one or more nodes may update respective copies of the distributed ledger with the record. In some embodiments, the one or more nodes (or a select group of nodes in the network) may use the consensus protocol to independently validate and/or verify the record.

In this way, the cognitive intelligence platform 102 adds a verifiable record of the other user viewing the care plan to the distributed ledger (and/or the distributed file system) and distributes the verifiable record to the one or more other nodes in the network.

FIG. 33E illustrates a transaction involving storing an updated care plan. In some embodiments, and as shown by reference number 3340, the cognitive intelligence platform 102 may receive, from the user device 104-1, a transaction request to store the updated care plan. For example, the medical professional may update the care plan (e.g., to improve upon one or more aspects of the plan) and may interact with an interface of the user device 104-1 to submit a transaction request to store the updated care plan. The transaction request may include the updated care plan, the credential information of the medical professional (e.g., shown as authorization information), the blockchain identifier associated with the blockchain used to store records associated with the care plan, a transaction identifier that indicates a type of transaction being requested, and/or the like.

In some embodiments, and as shown by reference number 3342, the cognitive intelligence platform 102 may determine whether to allow one or more operations to be performed to add the updated care plan to the distributed ledger. In some embodiments, and as shown by reference number 3344, the cognitive intelligence platform 102 may generate and add a record of the transaction to update the care plan to the distributed ledger.

In some embodiments, and as shown by reference number 3346, the cognitive intelligence platform 102 may provide a transaction response to the user device 104-1. The transaction response may indicate whether the updated care plan has been stored using the distributed ledger.

In some embodiments, and as shown by reference number 3348, the cognitive intelligence platform 102 may provide, to the one or more other nodes, the record of the transaction to add the updated care plan to the distributed ledger. In some embodiments, and as shown by reference number 3350, the one or more other nodes may update respective copies of the distributed ledger with record.

In this way, the cognitive intelligence platform 102 manages content stored using the distributed ledger in a manner that is secure, distributed, verifiable, and incentive-driven. For example, security is provided by supporting the distributed ledger with a tamper-resistant data structure (e.g., the blockchain), by implementing various forms of authentication, by restricting access to the network of nodes to particular users/entities, and/or the like. To provide a few particular examples, the distributed ledger improves security by preserving an immutable record of content, by using cryptographic links between blocks in the blockchain (e.g., reducing the potential for unauthorized tampering of the content), and/or the like. Security is further improved as a result of nodes that have access to the distributed ledger independently verifying each transaction that is added to the blockchain. Moreover, use the distributed ledger provides failover protection in that a particular node may continue to operate in a situation where one or more other nodes that have access to the distributed ledger fail.

As indicated above, FIGS. 33A-33E are provided merely as an example. Other examples are possible and may differ from what was described with regard to FIGS. 33A-33E. For example, there may be additional devices and/or networks, fewer devices and/or networks, different devices and/or networks, or differently arranged devices and/or networks than those shown in FIGS. 33A-33E. Furthermore, two or more devices shown in FIGS. 33A-33E may be implemented within a single device, or a single device shown in FIGS. 33A-33E may be implemented as multiple, distributed devices. Additionally, or alternatively, a set of devices (e.g., one or more devices) of the one or more example embodiments described above may perform one or more functions described as being performed by another set of devices of the one or more example embodiments.

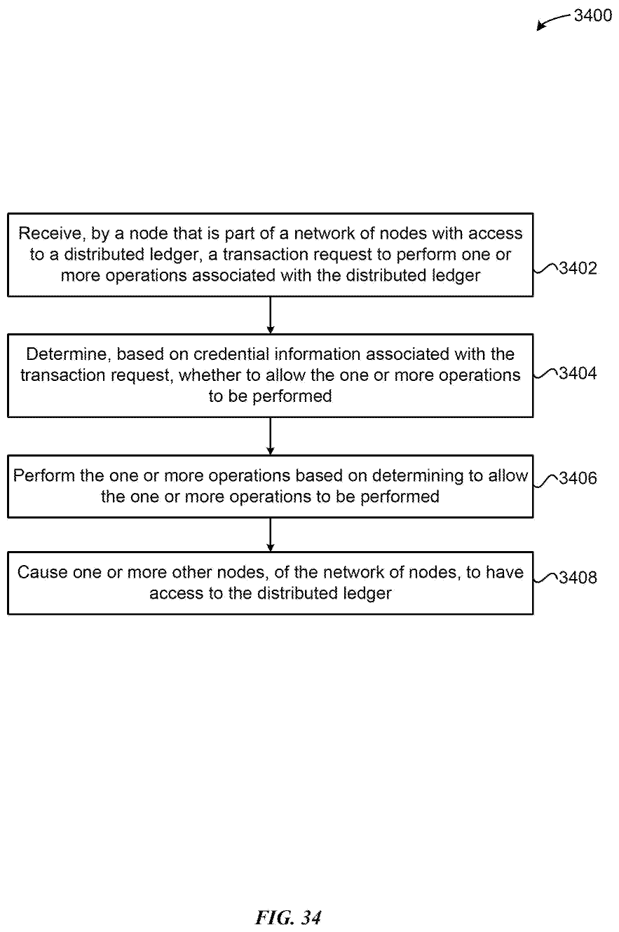

FIG. 34 shows an example method 3400 for managing content pertaining to healthcare in a distributed ledger, in accordance with various embodiments. In some embodiments, the method 3400 is implemented on a cognitive intelligence platform. In some embodiments, the cognitive intelligence platform is the cognitive intelligence platform 102 as shown in FIG. 1. In some embodiments, the cognitive intelligence platform 102 is implemented on the computing device 1400 shown in FIG. 14. The method 3400 may include operations that are implemented in computer instructions stored in a memory and executed by a processor of a computing device.

At block 3402, the method 3400 may include receiving, by a node that is part of a network of nodes with access to a distributed ledger, a transaction request to perform one or more operations associated with the distributed ledger. For example, the computing device 1400 may be part of a network of nodes with access to a distributed ledger and may (e.g., using processor 1402, input 1408, controller 1413, and/or the like) receive a transaction request to perform one or more operations associated with the distributed ledger, as described above. In some embodiments, the distributed ledger may be implemented by a blockchain.

At block 3404, the method 3400 may include determining, based on credential information associated with the transaction request, whether to allow the one or more operations to be performed. For example, the computing device 1400 (e.g., using processor 1402, controller 1413, and/or the like) may determine, based on credential information associated with the transaction request, whether to allow the one or more operations to be performed, as described above.

In some embodiments, the computing device 1400 may determine whether to allow the one or more operations to be performed by determining, using a credentials verification function of a smart contract, whether credential information of a user that caused the transaction request to be submitted matches or satisfies a threshold level of similarity with corresponding stored credential information associated with an authorized user. The computing device 1400 may determine whether to allow the one or more operations to be performed based on determining whether the credential information matches or satisfies the threshold level of similarity with the corresponding stored credential information.

In some embodiments, the computing device 1400 may determine whether to allow the one or more operations to be performed by determining, using an incentives function of a smart contract and information included in the transaction request, whether a quantity of electronic currency associated with a user that caused the transaction request to be submitted satisfies a threshold quantity of electronic currency needed to perform the one or more operations. The computing device 1400 may determine whether to allow the one or more operations to be performed based on determining whether the quantity of electronic currency associated with the user satisfies the threshold quantity of electronic currency.

At block 3406, the method 3400 may include performing the one or more operations based on determining to allow the one or more operations to be performed. For example, the computing device 1400 (e.g., using processor 1402, controller 1413, and/or the like) may perform the one or more operations based on determining to allow the one or more operations to be performed, as described above. In some embodiments, the one or more operations may include adding the record of the transaction to the distributed ledger.

In some embodiments, at least a subset of the content pertaining to healthcare may be included in the record. In some embodiments, the subset of the content included in the record may be a first subset and a distributed file system that is accessible to the network of nodes may be used to store a second subset of the content. In some embodiments, when performing the one or more operations, the computing device 1400 may identify an encrypted identifier associated with a user that submitted the transaction request. In some embodiments, when performing the one or more operations, the computing device 1400 may add the second subset of the content pertaining to healthcare to the distributed file system. In some embodiments, when performing the one or more operations, the computing device 1400 may generate the record of the transaction that includes the first subset of content. In some embodiments, when performing the one or more operations, the computing device 1400 may add the record of the transaction to the distributed ledger.

In some embodiments, the transaction request may indicate to store content using the distributed ledger. In some embodiments, when performing the one or more operations, the computing device 1400 may generate the record to include the content, wherein the content is a care plan of a medical professional or an updated care plan of the medical professional. In some embodiments, when performing the one or more operations, the computing device 1400 may add the record to the distributed ledger.

In some embodiments, the transaction request may be to view content that has been stored using the distributed ledger. In some embodiments, when performing the one or more operations, the computing device 1400 may generate the record to indicate that a user that submitted the transaction request has been approved to view the content. In some embodiments, when performing the one or more operations, the computing device 1400 may add the record to the distributed ledger. In some embodiments, when performing the one or more operations, the computing device 1400 may cause the content to be displayed on a device associated with the user that submitted the transaction request.

In some embodiments, when the transaction request may be requesting a level of trustworthiness of the content. In some embodiments, when performing the one or more operations, the computing device 1400 may determine a level of trustworthiness of the content based on at least one of verification data indicating that the content has been verified, or visibility data indicating a number of times other users have viewed the content. In some embodiments, when performing the one or more operations, the computing device 1400 may generate the record of the transaction to include the determined level of trustworthiness. In some embodiments, when performing the one or more operations, the computing device 1400 may add the record to the distributed ledger. In some embodiments, when performing the one or more operations, the computing device 1400 may cause a user device associated with a user that submitted the transaction request to display a representation of the level of trustworthiness of the content.

In some embodiments, the transaction request may be requesting a content recommendation. In some embodiments, when performing the one or more operations, the computing device 1400 may identify recommended content by using a ranking technique to process at least one of verification data or visibility data associated with a collection of content. In some embodiments, when performing the one or more operations, the computing device 1400 may generate the record of the transaction to include the recommended content. In some embodiments, when performing the one or more operations, the computing device 1400 may add the record to the distributed ledger. In some embodiments, when performing the one or more operations, the computing device 1400 may cause a user device associated with a user that submitted the transaction request to display the recommended content.

At block 3408, the method 3400 may include causing one or more other nodes, of the network of nodes, to have access to the distributed ledger. For example, the computing device 1400 (e.g., using processor 1402, network interface 1411, controller 1413, and/or the like) may cause one or more other nodes, of the network of nodes, to have access to the distributed ledger, as described above.
Clause 1. A method for maintaining content pertaining to healthcare in a distributed ledger, the method comprising:
receiving, by a node that is part of a network of nodes with access to the distributed ledger, a transaction request to perform one or more operations associated with the distributed ledger, wherein performing the one or more operations includes causing a record of a transaction to be stored using the distributed ledger;
determining, by the node and based on credential information associated with the transaction request, whether to allow the one or more operations to be performed;
performing, by the node, the one or more operations based on determining to allow the one or more operations to be performed, wherein the one or more operations include adding the record of the transaction to the distributed ledger, and wherein at least a subset of the content is included in the record; and
causing, by the node, one or more other nodes, of the network of nodes, to have access to the distributed ledger that includes the record.
Clause 2. The method of clause 1, wherein the content is a care plan of a medical professional or an updated care plan of the medical professional; and wherein at least a portion of the care plan or the updated care plan is generated by the node based on a machine-learning-driven analysis of a knowledge graph and a patient graph.
Clause 3. The method of clause 1, wherein determining whether to allow the one or more operations to be performed comprises:
determining, using an incentives function of a smart contract and information included in the transaction request, whether a quantity of electronic currency associated with a user that caused the transaction request to be submitted satisfies a threshold quantity of electronic currency needed to perform the one or more operations, and
determining whether to allow the one or more operations to be performed based on determining whether the quantity of electronic currency associated with the user satisfies the threshold quantity of electronic currency.
Clause 4. The method of clause 1, wherein the distributed ledger is implemented by a blockchain.
Clause 5. The method of clause 1, wherein at least the subset of the content included in the record is a first subset of the content pertaining to healthcare; wherein a distributed file system that is accessible to the network of nodes is used to store a second subset of the content; and wherein performing the one or more operations comprises:
identifying an encrypted identifier associated with a user that caused the transaction request to be submitted,
adding the second subset of the content pertaining to healthcare to the distributed file system,
generating the record of the transaction that includes the first subset of content, and
adding the record of the transaction to the distributed ledger.
Clause 6. The method of clause 1, wherein the transaction request indicates to store content using the distributed ledger; and wherein performing the one or more operations comprises:
generating the record to include the content, wherein the content is a care plan of a medical professional or an updated care plan of the medical professional, and
adding the record to the distributed ledger.
Clause 7. The method of clause 1, wherein the transaction request is to view content that has been stored using the distributed ledger; and wherein performing the one or more operations comprises:
generating the record to indicate that a user that submitted the transaction request has been approved to view the content,
adding the record to the distributed ledger, and
causing the content to be displayed on a device associated with the user that submitted the transaction request.
Clause 8. A node that is part of a network of nodes with access to a distributed ledger, comprising one or more processors; and one or more memories instructions that, when executed by the one or more processors, cause the one or more processors to:
receive a transaction request to perform one or more operations associated with the distributed ledger, wherein performing the one or more operations includes causing a record of a transaction to be stored using the distributed ledger;
determine, based on credential information associated with the transaction request, whether to allow the one or more operations to be performed;
perform the one or more operations based on determining to allow the one or more operations to be performed, wherein the one or more operations include adding the record of the transaction to the distributed ledger, and wherein at least a subset of content pertaining to healthcare is included in the record; and
cause one or more other nodes, of the network of nodes, to have access to the distributed ledger that includes the record.
Clause 9. The node of clause 8, wherein the content is a care plan of a medical professional or an updated care plan of the medical professional; and wherein the one or more processors are further to:
generate at least a portion of the care plan or the updated care plan based on a machine-learning-driven analysis of a knowledge graph and a patient graph.
Clause 10. The node of clause 8, wherein the one or more processors, when determining whether to allow the one or more operations to be performed, are to:
determine, using an incentives function of a smart contract and information included in the transaction request, whether a quantity of electronic currency associated with a user that caused the transaction request to be submitted satisfies a threshold quantity of electronic currency needed to perform the one or more operations, and
determine whether to allow the one or more operations to be performed based on determining whether the quantity of electronic currency associated with the user satisfies the threshold quantity of electronic currency.
Clause 11. The node of clause 8, wherein the distributed ledger is implemented by a blockchain.
Clause 12. The node of clause 8, wherein at least the subset of the content included in the record is a first subset of the content pertaining to healthcare; wherein a distributed file system that is accessible to the network of nodes is used to store a second subset of the content; and wherein the one or more processors, when performing the one or more operations, are to:
identify an encrypted identifier associated with a user that caused the transaction request to be submitted,
add the second subset of the content pertaining to healthcare to the distributed file system,
generate the record of the transaction that includes the first subset of content,
add the record of the transaction to the distributed ledger.
Clause 13. The node of clause m 8, wherein the transaction request indicates to store content using the distributed ledger; wherein the one or more processors, when performing the one or more operations, are to:
generate the record to include the content, wherein the content is a care plan of a medical professional or an updated care plan of the medical professional, and
add the record to the distributed ledger.
Clause 14. The node of clause 8, wherein the transaction request is requesting a content recommendation; and wherein the one or more processors, when performing the one or more operations, are to:
identify recommended content by using a ranking technique to process at least one of verification data or visibility data associated with a collection of content,
generate the record of the transaction to include the recommended content,
add the record to the distributed ledger, and
cause a user device associated with a user that submitted the transaction request to display the recommended content.
Clause 15. A non-transitory computer-readable medium storing instructions, the instructions comprising one or more instructions that, when executed by one or more processors, cause the one or more processors to:
receive a transaction request to perform one or more operations associated with the distributed ledger, wherein performing the one or more operations include causing a record of a transaction to be stored using the distributed ledger;
determine, based on credential information associated with the transaction request, whether to allow the one or more operations to be performed;
perform the one or more operations based on determining to allow the one or more operations to be performed, wherein the one or more operations include adding the record of the transaction to the distributed ledger, and wherein at least a subset of content pertaining to healthcare is included in the record; and
cause one or more other nodes, of the network of nodes, to have access to the distributed ledger that includes the record.
Clause 16. The non-transitory computer-readable medium of clause 15, wherein the one or more instructions, that cause the one or more processors to determine whether to allow the one or more operations to be performed, cause the one or more processors to:
determine, using a credentials verification function of a smart contract, whether credential information of a user that caused the transaction request to be submitted matches or satisfies a threshold level of similarity with corresponding stored credential information associated with an authorized user, and
determine whether to allow the one or more operations to be performed based on determining whether the credential information matches or satisfies the threshold level of similarity with the corresponding stored credential information.
Clause 17. The non-transitory computer-readable medium of clause 15, wherein the one or more instructions, that cause the one or more processors to determine whether to allow the one or more operations to be performed, cause the one or more processors to:
determine, using an incentives function of a smart contract and information included in the transaction request, whether a quantity of electronic currency associated with a user that caused the transaction request to be submitted satisfies a threshold quantity of electronic currency needed to perform the one or more operations, and
determine whether to allow the one or more operations to be performed based on determining whether the quantity of electronic currency associated with the user satisfies the threshold quantity of electronic currency.
Clause 18. The non-transitory computer-readable medium of clause 15, wherein the distributed ledger is implemented by a blockchain.
Clause 19. The non-transitory computer-readable medium of clause 15, wherein at least the subset of the content included in the record is a first subset of the content pertaining to healthcare; wherein a distributed file system that is accessible to the network of nodes is used to store a second subset of the content; and wherein the one or more instructions, that cause the one or more processors to perform the one or more operations, cause the one or more processors to:
identify an encrypted identifier associated with a user that caused the transaction request to be submitted,
add the second subset of the content pertaining to healthcare to the distributed file system,
generate the record of the transaction that includes the first subset of content, and
add the record of the transaction to the distributed ledger.
Clause 20. The non-transitory computer-readable medium of clause 15, wherein the transaction request is requesting a level of trustworthiness of the content; and wherein the one or more instructions, that cause the one or more processors to perform the one or more operations, cause the one or more processors to:
determine a level of trustworthiness of the content based on at least one of:
verification data indicating that the content has been verified, or
visibility data indicating a number of times other users have viewed the content,
generate the record of the transaction to include the determined level of trustworthiness,
add the record to the distributed ledger, and
cause a user device associated with a user that submitted the transaction request to display a representation of the level of trustworthiness of the content.
Clause 21. A method for maintaining content pertaining to healthcare in a distributed ledger, the method comprising:
receiving, from a computing device associated with a medical personnel entity, a transaction request to perform an operation on the distributed ledger, wherein the operation comprises storing content pertaining to healthcare in the distributed ledger;
executing one or more rules of the distributed ledger to determine whether to allow the operation to be performed, wherein at least one of the one or more rules comprises determining whether the medical personnel entity is associated with authorization information pertaining to healthcare; and
responsive to determining that the one or more rules of the distributed ledger are satisfied, performing the operation on the distributed ledger to store the content in the distributed ledger.
Clause 22. The method of clause 21, wherein the authorization information is stored as part of a record of the distributed ledger or the authorization information is included in the transaction request.
Clause 23. The method of clause 21, wherein the one or more rules comprise determining, using one or more transactions stored in the distributed ledger, whether at least a portion of the content is new relative to other content in the distributed ledger.
Clause 24. The method of clause 21, wherein the authorization information comprises a national provider identifier, and the one or more rules comprise determining, using one or more transactions stored in the distributed ledger, whether the authorization information is valid.
Clause 25. The method of clause 21, further comprising validating authentication information, authorization information, or both prior to allowing the content to be stored in the distributed ledger, wherein the authentication information are provided to the computing device during registration and the authorization information pertain to a medical license.
Clause 26. The method of clause 21, further comprising, responsive to determining that the one or more rules of the distributed ledger are not satisfied, preventing the operation from being performed on the distributed ledger.
Clause 27. The method of clause 21, wherein the content comprises a care plan, wherein at least a portion of the care plan is written by the medical personnel entity.
Clause 28. The method of clause 21, further comprising:
receiving, from the computing device, a second transaction request to perform a second operation on the distributed ledger, wherein the second transaction request comprises search criteria, and the second operation comprises providing, based on the search criteria, a care plan pertaining to healthcare that is stored in the distributed ledger;
executing the one or more rules of the distributed ledger to determine whether to allow the operation to be performed, wherein at least a second rule of the one or more rules comprises determining whether the medical personnel entity has a right to access the content; and
responsive to determining that the one or more rules of the distributed ledger are satisfied, performing the second operation on the distributed ledger to provide, based on the search criteria, the care plan pertaining to healthcare to the computing device.
Clause 29. A method comprising:
receiving, from the computing device associated, a second transaction request to perform a second operation on the distributed ledger, wherein the second operation comprises storing updated content pertaining to healthcare in the distributed ledger, and the updated document adds additional content to original content included in the content stored in the distributed ledger;
executing the one or more rules of the distributed ledger to determine whether to allow the operation to be performed, wherein at least a second rule of the one or more rules comprises determining whether the additional content in the updated content is new relative to other content pertaining to healthcare stored in the distributed ledger; and
responsive to determining that the one or more rules of the distributed ledger are satisfied, performing the second operation on the distributed ledger to store, in the distributed ledger, the updated content including the additional content and the original content.
Clause 30. The method of a previous clause, further comprising maintaining a time series for the content that indicates when the content was stored, modified, validated, or some combination thereof.
Clause 31. The method of a previous clause, further comprising:
receiving, from a computing device associated with a second medical personnel entity, a second transaction request to perform a second operation on the distributed ledger, wherein the second operation comprises verifying the content in the distributed ledger;
determining whether the second medical personnel entity is associated with valid authorization information; and
responsive to determining that the second medical personnel entity is associated with the valid authoring credential, performing the second operation on the distributed ledger by allowing the second medical personnel entity to verify the content.
Clause 32. The method of a previous clause, further comprising:
receiving, from a second computing device, a transaction request to perform a second operation on the distributed ledger, wherein the second operation comprises determining whether the content is trustworthy;
determining, using the distributed ledger, a source of the content;
determining that the source is associated with the authorization information;
determining whether the content has been verified within a certain time period; and
providing a notification to the second computing device that the content is trustworthy based on the source of the content being associated with the authorization information and the content being verified within the certain time period.
Clause 33. The method of a previous clause, wherein the content comprises a care plan for a medical issue, the medical personnel entity is a licensed medical doctor, and the distributed ledger maintains different care plans for different licensed medical doctors that are validated as being provided by licensed medical doctors based on authorization information that is stored in the distributed ledger.
Clause 34. A method for maintaining content pertaining to healthcare in a distributed ledger, the method comprising:
receiving, from a computing device associated with a medical personnel entity, a transaction request to perform an operation on the distributed ledger, wherein the transaction request comprises search criteria, and the operation comprises providing, based on the search criteria, content pertaining to healthcare that is stored in the distributed ledger;
executing one or more rules of the distributed ledger to determine whether to allow the operation to be performed, wherein at least one of the one or more rules comprises determining whether the medical personnel entity has a right to access the content; and
responsive to determining that the one or more rules of the distributed ledger are satisfied, performing the operation on the distributed ledger to provide, based on the search criteria, the content pertaining to healthcare to the computing device.
Clause 35. The method of a previous clause, further comprising:
receiving, from the computing device, a second transaction request to perform a second operation on the distributed ledger, wherein the second operation comprises storing second content pertaining to healthcare in the distributed ledger;
executing the one or more rules of the distributed ledger to determine whether to allow the second operation to be performed, wherein at least a second rule of the one or more rules comprises determining whether the medical personnel entity is associated with authorization information pertaining to healthcare; and
responsive to determining that the one or more rules of the distributed ledger are satisfied, performing the second operation on the distributed ledger to store the second content in the distributed ledger.
Clause 36. The method of a previous clause, wherein the right comprises the medical personnel entity being included in an organization that has viewing privileges of the content, the medical personnel entity being associated with authorization information pertaining to healthcare, or some combination thereof.
Clause 37. The method of a previous clause, further comprising:
receiving, from the computing device, a second transaction request to perform a second operation on the distributed ledger, wherein the second operation comprises storing updated content pertaining to healthcare in the distributed ledger, and the updated content adds additional content to original content included in the content stored in the distributed ledger;
executing the one or more rules of the distributed ledger to determine whether to allow the operation to be performed, wherein at least a second rule of the one or more rules comprises determining whether the additional content in the updated content is new relative to other content pertaining to healthcare stored in the distributed ledger; and
responsive to determining that the one or more rules of the distributed ledger are satisfied, performing the second operation on the distributed ledger to store, in the distributed ledger, the updated content including the additional content and the original content.
Clause 38. A method for maintaining content pertaining to healthcare in a distributed ledger, the method comprising:
receiving, from a computing device associated with a medical personnel entity, a transaction request to perform an operation on the distributed ledger, wherein the operation comprises storing updated content pertaining to healthcare in the distributed ledger, and the updated content adds additional content to original content stored in the distributed ledger;
executing one or more rules of the distributed ledger to determine whether to allow the operation to be performed, wherein at least one of the one or more rules comprises determining whether the additional content in the updated content is new relative to other content pertaining to healthcare stored in the distributed ledger; and
responsive to determining that the one or more rules of the distributed ledger are satisfied, performing the operation on the distributed ledger to store, in the distributed ledger, the updated content including the additional content and the original content.
Clause 39. The method of a previous clause, further comprising:
receiving, from the computing device, a second transaction request to perform a second operation on the distributed ledger, wherein the second operation comprises storing second content pertaining to healthcare in the distributed ledger;
executing the one or more rules of the distributed ledger to determine whether to allow the operation to be performed, wherein at least a second rule of the one or more rules comprises determining whether the medical personnel entity is associated with authorization information pertaining to healthcare; and
responsive to determining that the one or more rules of the distributed ledger are satisfied, performing the second operation on the distributed ledger to store the second content in the distributed ledger.
Clause 40. The method of a previous clause, further comprising:
receiving, from the computing device, a second transaction request to perform a second operation on the distributed ledger, wherein the second transaction request comprises search criteria, and the second operation comprises providing, based on the search criteria, the updated content pertaining to healthcare that is stored in the distributed ledger;
executing the one or more rules of the distributed ledger to determine whether to allow the second operation to be performed, wherein at least a second rule of the one or more rules comprises determining whether the medical personnel entity has a right to access the updated content; and
responsive to determining that the one or more rules of the distributed ledger are satisfied, performing the second operation on the distributed ledger to provide, based on the search criteria, the updated content pertaining to healthcare to the computing device.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

The word “example” is used herein to mean serving as an example, instance, or illustration. Any aspect or design described herein as “example” is not necessarily to be construed as preferred or advantageous over other aspects or designs. Rather, use of the word “example” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an implementation” or “one implementation” throughout is not intended to mean the same embodiment or implementation unless described as such.

Furthermore, as used herein, the term “set” is intended to include one or more items (e.g., related items, unrelated items, a combination of related and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

Some implementations are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, or the like.

Certain user interfaces have been described herein and/or shown in the figures. A user interface may include a graphical user interface, a non-graphical user interface, a text-based user interface, or the like. A user interface may provide information for display. In some implementations, a user may interact with the information, such as by providing input via an input component of a device that provides the user interface for display. In some implementations, a user interface may be configurable by a device and/or a user (e.g., a user may change the size of the user interface, information provided via the user interface, a position of information provided via the user interface, etc.). Additionally, or alternatively, a user interface may be pre-configured to a standard configuration, a specific configuration based on a type of device on which the user interface is displayed, and/or a set of configurations based on capabilities and/or specifications associated with a device on which the user interface is displayed.

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

Implementations the systems, algorithms, methods, instructions, etc., described herein can be realized in hardware, software, or any combination thereof. The hardware can include, for example, computers, intellectual property (IP) cores, application-specific integrated circuits (ASICs), programmable logic arrays, optical processors, programmable logic controllers, microcode, microcontrollers, servers, microprocessors, digital signal processors, or any other suitable circuit. In the claims, the term “processor” should be understood as encompassing any of the foregoing hardware, either singly or in combination. The terms “signal” and “data” are used interchangeably.

As used herein, the term module can include a packaged functional hardware unit designed for use with other components, a set of instructions executable by a controller (e.g., a processor executing software or firmware), processing circuitry configured to perform a particular function, and a self-contained hardware or software component that interfaces with a larger system. For example, a module can include an application specific integrated circuit (ASIC), a Field Programmable Gate Array (FPGA), a circuit, digital logic circuit, an analog circuit, a combination of discrete circuits, gates, and other types of hardware or combination thereof. In other embodiments, a module can include memory that stores instructions executable by a controller to implement a feature of the module.

Further, in one aspect, for example, systems described herein can be implemented using a general-purpose computer or general-purpose processor with a computer program that, when executed, carries out any of the respective methods, algorithms, and/or instructions described herein. In addition, or alternatively, for example, a special purpose computer/processor can be utilized which can contain other hardware for carrying out any of the methods, algorithms, or instructions described herein.

Further, all or a portion of implementations of the present disclosure can take the form of a computer program product accessible from, for example, a computer-usable or computer-readable medium. A computer-usable or computer-readable medium can be any device that can, for example, tangibly contain, store, communicate, or transport the program for use by or in connection with any processor. The medium can be, for example, an electronic, magnetic, optical, electromagnetic, or a semiconductor device. Other suitable mediums are also available.

The above-described embodiments, implementations, and aspects have been described in order to allow easy understanding of the present invention and do not limit the present invention. On the contrary, the invention is intended to cover various modifications and equivalent arrangements included within the scope of the appended claims, which scope is to be accorded the broadest interpretation to encompass all such modifications and equivalent structure as is permitted under the law.

What is claimed is:

1. A method for managing content pertaining to healthcare in a distributed ledger, the method comprising:
receiving, by a node that is part of a network of nodes with access to the distributed ledger, a transaction request to perform one or more operations associated with the distributed ledger,
wherein performing the one or more operations includes causing a record of a transaction to be stored using the distributed ledger;
determining, by the node and based on credential information associated with the transaction request, whether to allow the one or more operations to be performed;
performing, by the node, the one or more operations based on determining to allow the one or more operations to be performed,
wherein the one or more operations include adding the record of the transaction to the distributed ledger, and
wherein at least a subset of the content is included in the record; and
causing, by the node, one or more other nodes, of the network of nodes, to have access to the distributed ledger that includes the record.

2. The method of claim 1, wherein the content is a care plan of a medical professional or an updated care plan of the medical professional; and wherein at least a portion of the care plan or the updated care plan is generated by the node based on a machine-learning-driven analysis of a knowledge graph and a patient graph.

3. The method of claim 1, wherein determining whether to allow the one or more operations to be performed comprises:
determining, using an incentives function of a smart contract and information included in the transaction request, whether a quantity of electronic currency associated with a user that caused the transaction request to be submitted satisfies a threshold quantity of electronic currency needed to perform the one or more operations, and
determining whether to allow the one or more operations to be performed based on determining whether the quantity of electronic currency associated with the user satisfies the threshold quantity of electronic currency.

4. The method of claim 1, wherein the distributed ledger is implemented by a blockchain.

5. The method of claim 1, wherein at least the subset of the content included in the record is a first subset of the content pertaining to healthcare; wherein a distributed file system that is accessible to the network of nodes is used to store a second subset of the content; and
wherein performing the one or more operations comprises:
identifying an encrypted identifier associated with a user that caused the transaction request to be submitted,
adding the second subset of the content pertaining to healthcare to the distributed file system,
generating the record of the transaction that includes the first subset of content, and
adding the record of the transaction to the distributed ledger.

6. The method of claim 1, wherein the transaction request indicates to store content using the distributed ledger; and
wherein performing the one or more operations comprises:
generating the record to include the content, wherein the content is a care plan of a medical professional or an updated care plan of the medical professional, and
adding the record to the distributed ledger.

7. The method of claim 1, wherein the transaction request is to view content that has been stored using the distributed ledger; and
wherein performing the one or more operations comprises:
generating the record to indicate that a user that submitted the transaction request has been approved to view the content,
adding the record to the distributed ledger, and
causing the content to be displayed on a device associated with the user that submitted the transaction request.

8. A node that is part of a network of nodes in a distributed ledger, comprising:
one or more processors; and
one or more memories including instructions that, when executed by the one or more processors, cause the one or more processors to:
receive a transaction request to perform one or more operations associated with the distributed ledger,
wherein performing the one or more operations includes causing a record of a transaction to be stored using the distributed ledger;
determine, based on credential information associated with the transaction request, whether to allow the one or more operations to be performed;
perform the one or more operations based on determining to allow the one or more operations to be performed,
wherein the one or more operations include adding the record of the transaction to the distributed ledger, and
wherein at least a subset of content pertaining to healthcare is included in the record; and
cause one or more other nodes, of the network of nodes, to have access to the distributed ledger that includes the record.

9. The node of claim 8, wherein the content is a care plan of a medical professional or an updated care plan of the medical professional; and
wherein the one or more processors are further to:
generate at least a portion of the care plan or the updated care plan based on a machine-learning-driven analysis of a knowledge graph and a patient graph.

10. The node of claim 8, wherein the one or more processors, when determining whether to allow the one or more operations to be performed, are to:
determine, using an incentives function of a smart contract and information included in the transaction request, whether a quantity of electronic currency associated with a user that caused the transaction request to be submitted satisfies a threshold quantity of electronic currency needed to perform the one or more operations, and
determine whether to allow the one or more operations to be performed based on determining whether the quantity of electronic currency associated with the user satisfies the threshold quantity of electronic currency.

11. The node of claim 8, wherein the distributed ledger is implemented by a blockchain.

12. The node of claim 8, wherein at least the subset of the content included in the record is a first subset of the content pertaining to healthcare; wherein a distributed file system that is accessible to the network of nodes is used to store a second subset of the content; and
wherein the one or more processors, when performing the one or more operations, are to:
identify an encrypted identifier associated with a user that caused the transaction request to be submitted,
add the second subset of the content pertaining to healthcare to the distributed file system,
generate the record of the transaction that includes the first subset of content, and
add the record of the transaction to the distributed ledger.

13. The node of claim 8, wherein the transaction request indicates to store content using the distributed ledger; and
wherein the one or more processors, when performing the one or more operations, are to:
generate the record to include the content, wherein the content is a care plan of a medical professional or an updated care plan of the medical professional, and
add the record to the distributed ledger.

14. The node of claim 8, wherein the transaction request is requesting a content recommendation; and
wherein the one or more processors, when performing the one or more operations, are to:
identify recommended content by using a ranking technique to process at least one of verification data or visibility data associated with a collection of content,
generate the record of the transaction to include the recommended content,
add the record to the distributed ledger, and
cause a user device associated with a user that submitted the transaction request to display the recommended content.

15. A non-transitory computer-readable medium storing instructions, the instructions comprising:
one or more instructions that, when executed by one or more processors, cause the one or more processors to:
receive a transaction request to perform one or more operations associated with the distributed ledger,
wherein performing the one or more operations include causing a record of a transaction to be stored using the distributed ledger;
determine, based on credential information associated with the transaction request, whether to allow the one or more operations to be performed;
perform the one or more operations based on determining to allow the one or more operations to be performed,
wherein the one or more operations include adding the record of the transaction to the distributed ledger, and
wherein at least a subset of content pertaining to healthcare is included in the record; and
cause one or more other nodes, of the network of nodes, to have access to the distributed ledger that includes the record.

16. The non-transitory computer-readable medium of claim 15, wherein the one or more instructions, that cause the one or more processors to determine whether to allow the one or more operations to be performed, cause the one or more processors to:
determine, using a credentials verification function of a smart contract, whether credential information of a user that caused the transaction request to be submitted matches or satisfies a threshold level of similarity with corresponding stored credential information associated with an authorized user, and
determine whether to allow the one or more operations to be performed based on determining whether the credential information matches or satisfies the threshold level of similarity with the corresponding stored credential information.

17. The non-transitory computer-readable medium of claim 15, wherein the one or more instructions, that cause the one or more processors to determine whether to allow the one or more operations to be performed, cause the one or more processors to:
determine, using an incentives function of a smart contract and information included in the transaction request, whether a quantity of electronic currency associated with a user that caused the transaction request to be submitted satisfies a threshold quantity of electronic currency needed to perform the one or more operations, and
determine whether to allow the one or more operations to be performed based on determining whether the quantity of electronic currency associated with the user satisfies the threshold quantity of electronic currency.

18. The non-transitory computer-readable medium of claim 15, wherein the distributed ledger is implemented by a blockchain.

19. The non-transitory computer-readable medium of claim 15, wherein at least the subset of the content included in the record is a first subset of the content pertaining to healthcare; wherein a distributed file system that is accessible to the network of nodes is used to store a second subset of the content; and
wherein the one or more instructions, that cause the one or more processors to perform the one or more operations, cause the one or more processors to:
identify an encrypted identifier associated with a user that caused the transaction request to be submitted,
add the second subset of the content pertaining to healthcare to the distributed file system,
generate the record of the transaction that includes the first subset of content, and
add the record of the transaction to the distributed ledger.

20. The non-transitory computer-readable medium of claim 15, wherein the transaction request is requesting a level of trustworthiness of the content;
wherein the one or more instructions, that cause the one or more processors to perform the one or more operations, cause the one or more processors to:
determine a level of trustworthiness of the content based on at least one of:
verification data indicating that the content has been verified, or
visibility data indicating a number of times other users have viewed the content,
generate the record of the transaction to include the determined level of trustworthiness,
add the record to the distributed ledger, and
cause a user device associated with a user that submitted the transaction request to display a representation of the level of trustworthiness of the content.
